(19) United States
(12) Patent Application Publication
(10) Pub. No.: US 2005/0287544 A1
(43) Pub. Date: Dec. 29, 2005

(54) Gene expression profiling of colon cancer with DNA arrays

(76) Inventors: Francois Bertucci, Marseille (FR); Remi Houlgatte, Marseille (FR); Daniel Birnbaum, Marseille (FR); Stephane Debono, Marseille (FR)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 11/000,688

(22) Filed: Dec. 1, 2004

Related U.S. Application Data
(60) Provisional application No. 60/525,987, filed on Dec. 1, 2003.

(51) Int. Cl. C12Q 1/68

(52) U.S. Cl. 435/6

(57) ABSTRACT

Differential gene expression associated with histopathologic features of colorectal disease can be performed with nucleic acid arrays. Such arrays can comprise a pool of polynucleotide sequences from colon tissues, and the detection of the overexpression or underexpression of polynucleotide sequences (or subsequences or complements thereof) from this pool can provide information relating to the detection, diagnosis, stage, classification, monitoring, prediction, prevention or treatment of colorectal disease.

This Application claims the benefit of co-pending U.S. provisional patent application Ser. No. 60/525,987, filed Dec. 1, 2003, the entire disclosure of which is herein incorporated by reference.

### SEQUENCE LISTING

The instant application contains a “lengthy” Sequence Listing which has been submitted via CD-R in lieu of a printed paper copy, and is hereby incorporated by reference in its entirety. Said CD-R, recorded on May 5, 2005, are labeled CRF, “Copy 1” and “Copy 2”, respectively, and each contains only one identical 3.63 Mb file NAMED 1423R03.APP.

### FIELD OF THE INVENTION

The present invention relates to polynucleotide analysis and, in particular, to polynucleotide expression profiling of colorectal carcinomas using arrays of polynucleotides.

### BACKGROUND

Colorectal carcinoma (CRC) is a frequent and deadly disease. Different groups of tumors have been defined according to aggressiveness, anatomical localization and putative genetic instability based on conventional histopathological and immunohistopathological analysis. However, these aforementioned diagnostic tools are not sufficient to accurately diagnose and predict survival. Gene expression microarrays improve these classifications and bring new insights on the underlying molecular mechanisms involved throughout colorectal tumorigenic progression.

Despite global scientific efforts to effectively treat colon cancer, little progress has been made during the last decade and colorectal cancer (CRC) remains one of the most frequent and deadly neoplasias in western countries. Current prognostic models based on histoclinical parameters inadequately describe the heterogeneity of CRC, and are not sufficient to predict prognosis and guide clinical treatment in the individual patients. Tumors with different genetic alteration with similar clinical presentation follow different evolutions. One goal of molecular analysis is to identify, among complex networks of genes involved in tumorigenic progression, markers that could differentiate subgroups of tumors with prognosis, hence providing physicians with a clinically useful diagnostic tool to treat individual patients based on molecular gene sets as previously described.

Previous studies have been largely focused on individual candidate genes of disease, contrasting with the molecular complexity of cancer. The multi-step progression of CRC is accompanied by a number of genetic alterations [KRAS, APC, P53 and mismatch repair (MMR) genes, WNT and TGF-alpha pathways] that accumulate and interact in heterogenous complex ways to exert their tumor promoting effects (Vogelstein, 1988; Fearon, 1990). Despite the large number of published studies, the clinical utility of these disparate observations and reports remain limited for CRC patients. For example, little is known about molecular alterations associated with the prognostic heterogeneity of disease or the microsatellite instability (MSI) phenotype, and no single molecular marker has been validated to accurately predict prognososis in clinical practice. New models based on a precise molecular understanding of disease are required to improve screening, diagnosis,treatment, and ultimately survival of patients.

DNA microarray technology allows the measure of the mRNA expression level of thousands of genes simultaneously in a single assay, thus providing a molecular definition of a sample adapted to address the combinatory and complex nature of cancers (Bertucci, 2001; Ramaswamy, 2002; Mohr, 2002). Gene expression profiling may reveal biologically and/or clinically relevant subgroups of tumors (Alizadeh, 2000; Garber, 2001; Kihara, 2001; Beer, 2002; Bertucci, 2002; Devilard, 2002; Singh, 2002) and significantly improve current mechanistic understanding of oncogenesis.

Gene expression profiling-based studies of CRC have so far compared normal to tumor tissue samples, or described the molecular heterogeniety in different stages of colorectal disease (Alon, 1999; Notterman, 2001; Lin, 2002; Backert, 1999; Zou, 2002; Agrawal, 2002; Kitahara, 2001; Williams, 2003; Tureci, 2003; Birkenkamp-Demtroder, 2002; Frederiksen, 2003), but none have directly addressed the issue of prognosis or MSI phenotype.

### SUMMARY OF THE INVENTION

DNA microarrays may be utilized to elucidate discrete gene sets to improve the prognostic classification of CRC, identify novel potential therapeutic targets of carcinogenesis, describe new diagnostic and/or prognostic markers, and guide physician decisions on appropriate patient care.

The invention thus provides a method for analyzing differential gene expression associated with histopathologic features of colorectal disease, comprising the detection of the overexpression or underexpression of a pool of polynucleotide sequences in colon tissues, said pool comprising all or part of the polynucleotide sequences, subsequences or complements thereof, selected from each of predefined polynucleotide sequence sets I through 644 set forth in Table 1.

The invention further provides a method or prognosis or diagnosis of colon cancer, or for monitoring the treatment of a subject with a colon cancer. This method comprises the steps of 1) obtaining colon tissue nucleic acids from a patient; and 2) detecting the overexpression or underexpression of a pool of polynucleotide sequences in colon tissues. The pool of polynuclestide sequences comprises all or part of the polynucleotide sequences, subsequences or complements thereof, selected from each of predefined polynucleotide sequnce sets 1 through 644, as set forth in Table 1.

The invention further provides a polynucleotide library, comprising a pool of polynucleotide sequences either overexpressed or underexpressed in colon tissue, said pool corresponding to all or part of the polynucleotide sequences of SEQ ID Nos. 1 through 1596.

The invention still further provides a method of detecting differential gene expression, comprises 1) obtaining a polynucleotide sample from a subject; 2) reacting said polynucleotide sample obtained in step (1) with a polynucleotide library of the invention; and 3) detecting the reaction product of step (2).

The invention still further provides a method of assigning a therapeutic regimen to subject with histopathological features of colorectal disease, comprising 1) classifying the subject as having a “poor prognosis” or a “good prognosis” on the basis of the method of differential gene expression analysis according to the invention, and 2) assigning the subject a therapeutic regimen. The therapeutic regimen will either (i) comprise no adjuvant chemotherapy if the subject is lymph node negative and is classified as having a good prognosis, or (ii) comprise chemotherapy if said patient has any other combination of lymph node status and expression profile.

### BRIEF DESCRIPTION OF THE FIGURES

FIGS. 1A-1C show global gene expression profiles in colorectal cancer and non-cancerous samples.

FIGS. 2A-2B show hierarchical classifications of tissue samples using genes which discriminate between normal and cancer samples.

FIGS. 3A-3C show hierarchical classifications of CRC tissue samples using genes that discriminate metastatic from non-metastatic samples, correlated with survival.

FIGS. 4A-4C show hierarchical classifications of CRC tissue samples using discriminator genes selected by supervised analyses based on lymph node status, MSI phenotype and location of tumors.

FIGS. 5A-5C show the analysis of NM23 protein expression in colorectal tissue samples using tissue microarrays.

### DETAILED DESCRIPTION OF THE INVENTION

The present invention relates to DNA array, technology which can be used to analyse the expression of numerous (e.g., ˜8,000) genes in cancerous and non-cancerous colon tissue or cell samples. Unsupervised hierarchical clustering can be used to identify putative gene expression patterns that are precisely correlated to subgroups of tumors; and these sub-groups are notably correlated to patient prognosis, disease aggressiveness, and survival. Supervised analysis can be used to identify several genes differentially expressed between normal and cancer samples, and delineated subgroups of colon cancer can be defined by histoclinical parameters, including clinical outcome (i.e., 5-year survival of 100% in a group and 40% in the other group, p<0.005), lymph node invasion, tumors from the right or left colon, and MSI phenotype. Discriminator genes are associated with various cellular processes. The most significant discriminatory genes and/or potential markers identified by the present invention were further validated at the protein level using immunohistochemistry (IHC) on sections of tissue microarrays (TMA) on 190 tumor and normal samples (see Examples below).

The invention thus provides a method for analyzing differential gene expression associated with histopathologic features of colorectal disease, e.g., colon tumors, in particular colon cancer. The method of the invention comprises the detection of the overexpression or underexpression of a pool of polynucleotide sequences in colon tissues. The pool of polynucleotide sequences corresponds to all or part of the polynucleotide sequences, subsequences or complements thereof, selected from each of predefined polynucleotide sequences sets set forth in Table 1 below.

TABLE 1

| Gene | Set | | | | | |
|---|---|---|---|---|---|---|
| symbol | No. | Image | Name | Seq3′ | Seq5′ | Ref |
| CAPG | 1 | 1012666 | capping protein (actin filament), | SEQ ID No: 1 | | SEQ ID No: 2 |
| | | | gelsolin-like | | | |
| DEK | 2 | 1016390 | dek oncogene (dna binding) | SEQ ID No: 3 | | SEQ ID No: 4 |
| DVL1 | 3 | 1030065 | dishevelled, dsh homolog 1 (drosophila) | SEQ ID No: 5 | | SEQ ID No: 6 |
| NOV | 4 | 1046837 | nephroblastoma overexpressed gene | SEQ ID No: 7 | | SEQ ID No: 8 |
| CD79A | 5 | 1056782 | cd79a antigen (immunoglobulin- | SEQ ID No: 9 | | SEQ ID No: 10 |
| | | | associated alpha) | | | |
| MGC27076 | 6 | 108249 | hypothetical protein mgc27076 | SEQ ID No: 11 | SEQ ID No: 12 | SEQ ID No: 13 |
| | 7 | 108274 | | SEQ ID No: 14 | | |
| | 8 | 108292 | | SEQ ID No: 15 | | |
| C1ORF28 | 9 | 108305 | chromosome 1 open reading frame 28 | SEQ ID No: 16 | SEQ ID No: 17 | SEQ ID No: 18 |
| MAP2K2 | 10 | 108370 | mitogen-activated protein kinase kinase 2 | SEQ ID No: 19 | SEQ ID No: 20 | SEQ ID No: 21 |
| LOC220115 | 11 | 108374 | hypothetical protein loc220115 | | SEQ ID No: 22 | |
| | 12 | 108399 | | SEQ ID No: 23 | | |
| HRB | 13 | 108490 | hiv-1 rev binding protein | SEQ ID No: 24 | | SEQ ID No: 25 |
| | 14 | 110385 | hypothetical gene supported by | SEQ ID No: 26 | SEQ ID No: 27 | |
| | | | ak026041 | | | |
| LOC92906 | 15 | 110486 | hypothetical protein bc008217 | SEQ ID No: 28 | SEQ ID No: 29 | SEQ ID No: 30 |
| SOX4 | 16 | 111461 | sry (sex determining region y)-box 4 | SEQ ID No: 31 | SEQ ID No: 32 | SEQ ID No: 33 |
| GSTA2 | 17 | 113932 | glutathione s-transferase a2 | SEQ ID No: 34 | SEQ ID No: 35 | SEQ ID No: 36 |
| MLLT3 | 18 | 1144752 | myeloid/lymphoid or mixed-lineage | SEQ ID No: 37 | | SEQ ID No: 38 |
| | | | leukemia (trithorax homolog, | | | |
| | | | drosophila); translocated to, 3 | | | |
| TCF3 | 19 | 114639 | transcription factor 3 (e2a | SEQ ID No: 39 | SEQ ID No: 40 | SEQ ID No: 41 |
| | | | immunoglobulin enhancer binding | | | |
| | | | factors e12/e47) | | | |
| PMS2 | 20 | 116906 | pms2 postmeiotic segregation increased | SEQ ID No: 42 | SEQ ID No: 43 | SEQ ID No: 44 |
| | | | 2 (s. cerevisiae) | | | |
| LPP | 21 | 117240 | lim domain containing preferred | SEQ ID No: 45 | SEQ ID No: 46 | SEQ ID No: 47 |
| | | | translocation partner in lipoma | | | |
| PTPRC | 22 | 117755 | protein tyrosine phosphatase, receptor | SEQ ID No: 48 | | SEQ ID No: 49 |
| | | | type, c | | | |
| | 23 | 117811 | similar to [human ig rearranged gamma | SEQ ID No: 50 | SEQ ID No: 51 | |
| | | | chain mrna, v-j-c region and complete | | | |
| | | | cds.], gene product | | | |
| C6ORF53 | 24 | 1184178 | chromosome 6 open reading frame 53 | SEQ ID No: 52 | | SEQ ID No: 53 |
| PDPK1 | 25 | 1185650 | 3-phosphoinositide dependent protein | SEQ ID No: 54 | | SEQ ID No: 55 |
| | | | kinase-1 | | | |
| | 26 | 118634 | similar to [human ig rearranged gamma | SEQ ID No: 56 | SEQ ID No: 57 | |
| | | | chain mrna, v-j-c region and complete | | | |
| | | | cds.], gene product | | | |
| KCNJ15 | 27 | 119530 | potassium inwardly-rectifying channel, | SEQ ID No: 58 | SEQ ID No: 59 | SEQ ID No: 60 |
| | | | subfamily j, member 15 | | | |
| | 28 | 119772 | loc284066 | SEQ IDNo: 61 | | |
| USP9X | 29 | 120009 | ubiquitin specific protease 9, x | SEQ ID No: 62 | SEQ ID No: 63 | SEQ ID No: 64 |
| | | | chromosome (fat facets-like drosophila) | | | |
| HELZ | 30 | 120572 | helicase with zinc finger domain | SEQ ID No: 65 | | SEQ ID No: 66 |
| ADD1 | 31 | 120783 | adducin 1 (alpha) | SEQ ID No: 67 | | SEQ ID No: 68 |
| ATP5L | 32 | 121076 | atp synthase, h+ transporting, | SEQ ID No: 69 | | SEQ ID No: 70 |
| | | | mitochondrial f0 complex, subunit g | | | |
| IFNAR1 | 33 | 121265 | interferon (alpha, beta and omega) | SEQ ID No: 71 | SEQ ID No: 72 | SEQ ID No: 73 |
| | | | receptor 1 | | | |
| ELAVL1 | 34 | 121366 | elav (embryonic lethal, abnormal | SEQ ID No: 74 | | SEQ ID No: 75 |
| | | | vision, drosophila)-like 1 (hu antigen r) | | | |
| | 35 | 122004 | loc143724 | SEQ ID No: 76 | | |
| DSG1 | 36 | 122743 | desmoglein 1 | SEQ ID No: 77 | SEQ ID No: 78 | SEQ ID No: 79 |
| OLFM1 | 37 | 122756 | olfactomedin 1 | SEQ ID No: 80 | | SEQ ID No: 81 |
| C3 | 38 | 123379 | complement component 3 | SEQ ID No: 82 | | SEQ ID No: 83 |
| C4BPA | 39 | 123664 | complement component 4 binding | SEQ ID No: 84 | SEQ ID No: 85 | SEQ ID No: 86 |
| | | | protein, alpha | | | |
| DMPK | 40 | 123916 | dystrophia myotonica-protein kinase | SEQ ID No: 87 | SEQ ID No: 88 | SEQ ID No: 89 |
| RPL6 | 41 | 123948 | ribosomal protein 16 | SEQ ID No: 90 | SEQ ID No: 91 | SEQ ID No: 92 |
| HLA-DQB1 | 42 | 123953 | major histocompatibility complex, class | SEQ ID No: 93 | SEQ ID No: 94 | SEQ ID No: 95 |
| | | | ii, dq beta 1 | | | |
| CENPF | 43 | 124345 | centromere protein f, 350/400 ka | SEQ ID No: 96 | SEQ ID No: 97 | SEQ ID No: 98 |
| | | | (mitosin) | | | |
| CSF1 | 44 | 124554 | colony stimulating factor 1 | SEQ ID No: 99 | | SEQ ID No: 100 |
| | | | (macrophage) | | | |
| NDST3 | 45 | 125806 | n-deacetylase/n-sulfotransferase | SEQ ID No: 101 | SEQ ID No: 102 | SEQ ID No: 103 |
| | | | (heparan glucosaminyl) 3 | | | |
| SPI1 | 46 | 127394 | spleen focus forming virus (sffv) | SEQ ID No: 104 | SEQ ID No: 105 | SEQ ID No: 106 |
| | | | proviral integration oncogene spi1 | | | |
| ATP5C1 | 47 | 127950 | atp synthase, h+ transporting, | SEQ ID No: 107 | SEQ ID No: 108 | SEQ ID No: 109 |
| | | | mitochondrial f1 complex, gamma | | | |
| | | | polypeptide 1 | | | |
| TNFSF10 | 48 | 128413 | tumor necrosis factor (ligand) | SEQ ID No: 110 | SEQ ID No: 111 | SEQ ID No: 112 |
| | | | superfamily, member 10 | | | |
| ASBABP2 | 49 | 129112 | aspecific bcl2 are-binding protein 2 | SEQ ID No: 113 | | SEQ ID No: 114 |
| COX7A2L | 50 | 129146 | cytochrome c oxidase subunit viia | SEQ ID No: 115 | SEQ ID No: 116 | SEQ ID No: 117 |
| | | | polypeptide 2 like | | | |
| XTP5 | 51 | 129227 | minor histocompatibility antigen ha-8 | SEQ ID No: 118 | SEQ ID No: 119 | SEQ ID No: 120 |
| GATA3 | 52 | 129757 | gata binding protein 3 | SEQ ID No: 121 | | SEQ ID No: 122 |
| STK6 | 53 | 129865 | serine/threonine kinase 6 | SEQ ID No: 123 | | SEQ ID No: 124 |
| FLJ14297 | 54 | 130173 | hypothetical protein flj14297 | SEQ ID No: 125 | SEQ ID No: 126 | SEQ ID No: 127 |
| HEYL | 55 | 132307 | hairy/enhancer-of-split related with | SEQ ID No: 128 | SEQ ID No: 129 | SEQ ID No: 130 |
| | | | yrpw motif-like | | | |
| CD2 | 56 | 1326652 | cd2 antigen (p50), sheep red blood cell | SEQ ID No: 131 | | SEQ ID No: 132 |
| | | | receptor | | | |
| GRF2 | 57 | 133334 | guanine nucleotide-releasing factor 2 | SEQ ID No: 133 | | SEQ ID No: 134 |
| | | | (specific for crk proto-oncogene) | | | |
| ITGAL | 58 | 1338831 | integrin, alpha 1 (antigen cd11a (p180), | SEQ ID No: 135 | | SEQ ID No: 136 |
| | | | lymphocyte function-associated antigen | | | |
| | | | 1; alpha polypeptide) | | | |
| SPIB | 59 | 1350545 | spi-b transcription factor (spi-1/pu.1 | SEQ ID No: 137 | | SEQ ID No: 138 |
| | | | related) | | | |
| S100P | 60 | 135221 | s100 calcium binding protein p | SEQ ID No: 139 | SEQ ID No: 140 | SEQ ID No: 141 |
| PVRL3 | 61 | 135302 | poliovirus receptor-related 3 | SEQ ID No: 142 | SEQ ID No: 143 | SEQ ID No: 144 |
| | 62 | 136361 | | SEQ ID No: 145 | SEQ ID No: 146 | |
| COX6A1 | 63 | 139069 | cytochrome c oxidase subunit via | SEQ ID No: 147 | SEQ ID No: 148 | SEQ ID No: 149 |
| | | | polypeptide 1 | | | |
| IL2RB | 64 | 139073 | interleukin 2 receptor, beta | SEQ ID No: 150 | SEQ ID No: 151 | SEQ ID No: 152 |
| CDK2 | 65 | 1391584 | cyclin-dependent kinase 2 | SEQ ID No: 153 | | SEQ ID No: 154 |
| GPR1 | 66 | 139304 | g protein-coupled receptor 1 | SEQ ID No: 155 | SEQ ID No: 156 | SEQ ID No: 157 |
| PSG6 | 67 | 139392 | pregnancy specific beta-1-glycoprotein 6 | SEQ ID No: 158 | SEQ ID No: 159 | SEQ ID No: 160 |
| EPS15 | 68 | 139789 | epidermal growth factor receptor | SEQ ID No: 161 | SEQ ID No: 162 | SEQ ID No: 163 |
| | | | pathway substrate 15 | | | |
| APRT | 69 | 141998 | adenine phosphoribosyltransferase | SEQ ID No: 164 | SEQ ID No: 165 | SEQ ID No: 166 |
| TGFB1I1 | 70 | 1423050 | transforming growth factor beta 1 | SEQ ID No: 167 | | SEQ ID No: 168 |
| | | | induced transcript 1 | | | |
| FKBP2 | 71 | 143519 | fk506 binding protein 2, 13 kda | SEQ ID No: 169 | SEQ ID No: 170 | SEQ ID No: 171 |
| | 72 | 144853 | | SEQ ID No: 172 | | |
| BLVRA | 73 | 145269 | biliverdin reductase a | SEQ ID No: 173 | SEQ ID No: 174 | SEQ ID No: 175 |
| SLC30A5 | 74 | 145286 | solute carrier family 30 (zinc | SEQ ID No: 176 | SEQ ID No: 177 | SEQ ID No: 178 |
| | | | transporter), member 5 | | | |
| AZGP1 | 75 | 1456160 | alpha-2-glycoprotein 1, zinc | SEQ ID No: 179 | | SEQ ID No: 180 |
| | 76 | 1456315 | homo sapiens cdna flj30452 fis, clone | SEQ ID No: 181 | | |
| | | | brace2009293. | | | |
| KLRD1 | 77 | 145696 | killer cell lectin-like receptor subfamily | | SEQ ID No: 182 | SEQ ID No: 183 |
| | | | d, member 1 | | | |
| FOLR2 | 78 | 146494 | folate receptor 2 (fetal) | SEQ ID No: 184 | SEQ ID No: 185 | SEQ ID No: 186 |
| | 79 | 146922 | | SEQ ID No: 187 | SEQ ID No: 188 | |
| PTGS2 | 80 | 147050 | prostaglandin-endoperoxide synthase 2 | SEQ ID No: 189 | SEQ ID No: 190 | SEQ ID No: 191 |
| | | | (prostaglandin g/h synthase and | | | |
| | | | cyclooxygenase) | | | |
| PECAM1 | 81 | 147341 | platelet/endothelial cell adhesion | SEQ ID No: 192 | | SEQ ID No: 193 |
| | | | molecule (cd31 antigen) | | | |
| PSEN1 | 82 | 147495 | presenilin 1 (alzheimer disease 3) | SEQ ID No: 194 | SEQ ID No: 195 | SEQ ID No: 196 |
| | 83 | 1493187 | homo sapiens, clone image: 4831215, | SEQ ID No: 197 | | |
| | | | mrna | | | |
| GATA2 | 84 | 149809 | gata binding protein 2 | SEQ ID No: 198 | SEQ ID No: 199 | SEQ ID No: 200 |
| CHST13 | 85 | 1500894 | carbohydrate (chondroitin 4) | SEQ ID No: 201 | | SEQ ID No: 202 |
| | | | sulfotransferase 13 | | | |
| IGF1R | 86 | 150361 | insulin-like growth factor 1 receptor | SEQ ID No: 203 | SEQ ID No: 204 | SEQ ID No: 205 |
| SOCS2 | 87 | 150644 | suppressor of cytokine signaling 2 | SEQ ID No: 206 | SEQ ID No: 207 | SEQ ID No: 208 |
| INSR | 88 | 151149 | insulin receptor | | SEQ ID No: 209 | SEQ ID No: 210 |
| TFDP1 | 89 | 151495 | transcription factor dp-1 | SEQ ID No: 211 | SEQ ID No: 212 | SEQ ID No: 213 |
| IL10RA | 90 | 151740 | interleukin 10 receptor, alpha | SEQ ID No: 214 | SEQ ID No: 215 | SEQ ID No: 216 |
| LYK5 | 91 | 152467 | protein kinase lyk5 | SEQ ID No: 217 | SEQ ID No: 218 | SEQ ID No: 219 |
| MYBL1 | 92 | 1526789 | v-myb myeloblastosis viral oncogene | SEQ ID No: 220 | | |
| | | | homolog (avian)-like 1 | | | |
| LIF | 93 | 153025 | leukemia inhibitory factor (cholinergic | SEQ ID No: 221 | SEQ ID No: 222 | SEQ ID No: 223 |
| | | | differentiation factor) | | | |
| EIF4G3 | 94 | 153141 | eukaryotic translation initiation factor 4 | SEQ ID No: 224 | SEQ ID No: 225 | SEQ ID No: 226 |
| | | | gamma, 3 | | | |
| TGFB1I1 | 95 | 153461 | transforming growth factor beta 1 | SEQ ID No: 227 | SEQ ID No: 228 | SEQ ID No: 168 |
| | | | induced transcript 1 | | | |
| TJP3 | 96 | 153474 | tight junction protein 3 (zona occludens | SEQ ID No: 229 | SEQ ID No: 230 | SEQ ID No: 231 |
| | | | 3) | | | |
| STC1 | 97 | 153589 | stanniocalcin 1 | SEQ ID No: 232 | SEQ ID No: 233 | SEQ ID No: 234 |
| DES | 98 | 153854 | desmin | SEQ ID No: 235 | SEQ ID No: 236 | SEQ ID No: 237 |
| FCGBP | 99 | 154172 | fc fragment of igg binding protein | SEQ ID No: 238 | | SEQ ID No: 239 |
| PMSCL2 | 100 | 154335 | polymyositis/scleroderma autoantigen | SEQ ID No: 240 | SEQ ID No: 241 | SEQ ID No: 242 |
| | | | 2, 100 kda | | | |
| PLCD1 | 101 | 154600 | phospholipase c, delta 1 | SEQ ID No: 243 | SEQ ID No: 244 | SEQ ID No: 245 |
| CRIP1 | 102 | 155219 | cysteine-rich protein 1 (intestinal) | SEQ ID No: 246 | | SEQ ID No: 247 |
| BCKDK | 103 | 155774 | branched chain alpha-ketoacid | SEQ ID No: 248 | SEQ ID No: 249 | SEQ ID No: 250 |
| | | | dehydrogenase kinase | | | |
| TCF3 | 104 | 156505 | transcription factor 3 (e2a | SEQ ID No: 251 | | SEQ ID No: 41 |
| | | | immunoglobulin enhancer binding | | | |
| | | | factors e12/e47) | | | |
| ZNF463 | 105 | 156718 | zinc finger protein 463 | SEQ ID No: 252 | | SEQ ID No: 253 |
| MCP | 106 | 158233 | membrane cofactor protein (cd46, | SEQ ID No: 254 | SEQ ID No: 255 | SEQ ID No: 256 |
| | | | trophoblast-lymphocyte cross-reactive | | | |
| | | | antigen) | | | |
| LTBP4 | 107 | 158239 | latent transforming growth factor beta | SEQ ID No: 257 | SEQ ID No: 258 | SEQ ID No: 259 |
| | | | binding protein 4 | | | |
| MEIS1 | 108 | 1591384 | meis1, myeloid ecotropic viral | SEQ ID No: 260 | | SEQ ID No: 261 |
| | | | integration site 1 homolog (mouse) | | | |
| ACE | 109 | 159885 | angiotensin i converting enzyme | SEQ ID No: 262 | | SEQ ID No: 263 |
| | | | (peptidyl-dipeptidase a) 1 | | | |
| CD3E | 110 | 159903 | cd3e antigen, epsilon polypeptide (tit3 | SEQ ID No: 264 | | SEQ ID No: 265 |
| | | | complex) | | | |
| MGC39325 | 111 | 165818 | hypothetical protein mgc39325 | SEQ ID No: 266 | SEQ ID No: 267 | SEQ ID No: 268 |
| PRKACA | 112 | 166052 | protein kinase, camp-dependent, | | SEQ ID No: 269 | SEQ ID No: 270 |
| | | | catalytic, alpha | | | |
| SERPINB5 | 113 | 1662274 | serine (or cysteine) proteinase inhibitor, | SEQ ID No: 271 | | SEQ ID No: 272 |
| | | | clade b (ovalbumin), member 5 | | | |
| HSF4 | 114 | 1667886 | heat shock transcription factor 4 | SEQ ID No: 273 | | SEQ ID No: 274 |
| DOK2 | 115 | 1671188 | docking protein 2, 56 kda | SEQ ID No: 275 | | SEQ ID No: 276 |
| EEF1A1 | 116 | 1683100 | eukaryotic translation elongation factor | SEQ ID No: 277 | | SEQ ID No: 278 |
| | | | 1 alpha 1 | | | |
| S100A12 | 117 | 1705397 | s100 calcium binding protein a12 | SEQ ID No: 279 | | SEQ ID No: 280 |
| | | | (calgranulin c) | | | |
| CAMK2B | 118 | 172444 | calcium/calmodulin-dependent protein | SEQ ID No: 281 | SEQ ID No: 282 | SEQ ID No: 283 |
| | | | kinase (cam kinase) ii beta | | | |
| PLCG2 | 119 | 1731982 | phospholipase c, gamma 2 | SEQ ID No: 284 | | SEQ ID No: 285 |
| | | | (phosphatidylinositol-specific) | | | |
| NME1 | 120 | 174388 | non-metastatic cells 1, protein (nm23a) | SEQ ID No: 286 | SEQ ID No: 287 | SEQ ID No: 288 |
| | | | expressed in | | | |
| PTGDS | 121 | 178305 | prostaglandin d2 synthase 21 kda (brain) | SEQ ID No: 289 | SEQ ID No: 290 | SEQ ID No: 291 |
| PP | 122 | 179232 | pyrophosphatase (inorganic) | SEQ ID No: 292 | | SEQ ID No: 293 |
| PPP2R2C | 123 | 179264 | protein phosphatase 2 (formerly 2a), | SEQ ID No: 294 | | |
| | | | regulatory subunit b (pr 52), gamma | | | |
| | | | isoform | | | |
| | 124 | 179776 | | SEQ ID No: 295 | | |
| | 125 | 181827 | | SEQ ID No: 296 | | |
| TP53 | 126 | 1847162 | tumor protein p53 (li-fraumeni | SEQ ID No: 297 | | SEQ ID No: 298 |
| | | | syndrome) | | | |
| DARS | 127 | 186331 | aspartyl-trna synthetase | SEQ ID No: 299 | SEQ ID No: 300 | SEQ ID No: 301 |
| EGF | 128 | 1869652 | epidermal growth factor (beta- | SEQ ID No: 302 | | SEQ ID No: 303 |
| | | | urogastrone) | | | |
| RPL29P2 | 129 | 190103 | ribosomal protein 129 pseudogene 2 | SEQ ID No: 304 | SEQ ID No: 305 | |
| EEF1B2 | 130 | 1902297 | eukaryotic translation elongation factor | SEQ ID No: 306 | | SEQ ID No: 307 |
| | | | 1 beta 2 | | | |
| STK6 | 131 | 1912132 | serine/threonine kinase 6 | SEQ ID No: 308 | | SEQ ID No: 124 |
| TAL1 | 132 | 191548 | t-cell acute lymphocytic leukemia 1 | SEQ ID No: 309 | | |
| RPS15A | 133 | 191714 | ribosomal protein s15a | SEQ ID No: 310 | | SEQ ID No: 311 |
| RPS19 | 134 | 192242 | ribosomal protein s19 | SEQ ID No: 312 | | SEQ ID No: 313 |
| HRD1 | 135 | 192515 | hrd1 protein | SEQ ID No: 314 | | SEQ ID No: 315 |
| PTPN21 | 136 | 192581 | protein tyrosine phosphatase, non- | SEQ ID No: 316 | | SEQ ID No: 317 |
| | | | receptor type 21 | | | |
| NDUFA4 | 137 | 193672 | nadh dehydrogenase (ubiquinone) 1 | SEQ ID No: 318 | SEQ ID No: 319 | SEQ ID No: 320 |
| | | | alpha subcomplex, 4, 9 kda | | | |
| TSG101 | 138 | 194350 | tumor susceptibility gene 101 | SEQ ID No: 321 | SEQ ID No: 322 | SEQ ID No: 323 |
| SDHD | 139 | 195013 | succinate dehydrogenase complex, | SEQ ID No: 324 | SEQ ID No: 325 | SEQ ID No: 326 |
| | | | subunit d, integral membrane protein | | | |
| DAP3 | 140 | 195702 | death associated protein 3 | SEQ ID No: 327 | SEQ ID No: 328 | SEQ ID No: 329 |
| BTF3 | 141 | 195889 | basic transcription factor 3 | SEQ ID No: 330 | | SEQ ID No: 331 |
| BUB3 | 142 | 198903 | bub3 budding uninhibited by | SEQ ID No: 332 | SEQ ID No: 333 | SEQ ID No: 334 |
| | | | benzimidazoles 3 homolog (yeast) | | | |
| | 143 | 199837 | homo sapiens transcribed sequence with | SEQ ID No: 335 | | |
| | | | strong similarity to protein sp: p08865 | | | |
| | | | (h. sapiens) rsp4_human 40s ribosomal | | | |
| | | | protein sa (p40) (34/67 kda laminin | | | |
| | | | receptor) (colon carcinoma laminin- | | | |
| | | | binding protein) (nem/1chd4) | | | |
| OAS1 | 144 | 200521 | 2′,5′-oligoadenylate synthetase 1, | SEQ ID No: 336 | SEQ ID No: 337 | SEQ ID No: 338 |
| | | | 40/46 kda | | | |
| CD209L | 145 | 200714 | cd209 antigen-like | SEQ ID No: 339 | SEQ ID No: 340 | SEQ ID No: 341 |
| FGB | 146 | 201352 | fibrinogen, b beta polypeptide | SEQ ID No: 342 | | SEQ ID No: 343 |
| MYL1 | 147 | 201925 | myosin, light polypeptide 1, alkali; | SEQ ID No: 344 | SEQ ID No: 345 | SEQ ID No: 346 |
| | | | skeletal, fast | | | |
| PRPF4B | 148 | 202609 | prp4 pre-mrna processing factor 4 | SEQ ID No: 347 | SEQ ID No: 348 | SEQ ID No: 349 |
| | | | homolog b (yeast) | | | |
| ARGBP2 | 149 | 203264 | arg/abl-interacting protein argbp2 | SEQ ID No: 350 | SEQ ID No: 351 | SEQ ID No: 352 |
| RFC4 | 150 | 203275 | replication factor c (activator 1) 4, | SEQ ID No: 353 | SEQ ID No: 354 | SEQ ID No: 355 |
| | | | 37 kda | | | |
| CSF1R | 151 | 204653 | colony stimulating factor 1 receptor, | SEQ ID No: 356 | SEQ ID No: 357 | SEQ ID No: 358 |
| | | | formerly mcdonough feline sarcoma | | | |
| | | | viral (v-fms) oncogene homolog | | | |
| | 152 | 204740 | | SEQ ID No: 359 | | |
| | 153 | 2048801 | homo sapiens mrna full length insert | SEQ ID No: 360 | | |
| | | | cdna clone euroimage 1630957 | | | |
| TP53 | 154 | 205314 | tumor protein p53 (li-fraumeni | | SEQ ID No: 361 | SEQ ID No: 298 |
| | | | syndrome) | | | |
| LRP2 | 155 | 2055272 | low density lipoprotein-related protein 2 | SEQ ID No: 362 | | SEQ ID No: 363 |
| SP110 | 156 | 205612 | sp110 nuclear body protein | SEQ ID No: 364 | SEQ ID No: 365 | SEQ ID No: 366 |
| CCNF | 157 | 206323 | cyclin f | | SEQ ID No: 367 | SEQ ID No: 368 |
| CAPN12 | 158 | 206522 | calpain 12 | SEQ ID No: 369 | SEQ ID No: 370 | |
| GRB14 | 159 | 2067776 | growth factor receptor-bound protein 14 | SEQ ID No: 371 | | SEQ ID No: 372 |
| DDX24 | 160 | 207491 | dead (asp-glu-ala-asp) box polypeptide | SEQ ID No: 373 | SEQ ID No: 374 | SEQ ID No: 375 |
| | | | 24 | | | |
| | 161 | 208357 | | SEQ ID No: 376 | SEQ ID No: 377 | |
| HPN | 162 | 208413 | hepsin (transmembrane protease, serine | SEQ ID No: 378 | SEQ ID No: 379 | SEQ ID No: 380 |
| | | | 1) | | | |
| MGP | 163 | 209710 | matrix gla protein | SEQ ID No: 381 | | SEQ ID No: 382 |
| | 164 | 2106469 | similar to riken cdna 4933405110 | SEQ ID No: 383 | | |
| EPB41L4B | 165 | 210698 | erythrocyte membrane protein band 4.1 | SEQ ID No: 384 | SEQ ID No: 385 | SEQ ID No: 386 |
| | | | like 4b | | | |
| RPS4X | 166 | 211433 | ribosomal protein s4, x-linked | SEQ ID No: 387 | | SEQ ID No: 388 |
| IGF2 | 167 | 211445 | insulin-like growth factor 2 | SEQ ID No: 389 | | SEQ ID No: 390 |
| | | | (somatomedin a) | | | |
| UBA52 | 168 | 211920 | ubiquitin a-52 residue ribosomal protein | SEQ ID No: 391 | SEQ ID No: 392 | SEQ ID No: 393 |
| | | | fusion product 1 | | | |
| AKR1C3 | 169 | 211995 | aldo-keto reductase family 1, member | | SEQ ID No: 394 | SEQ ID No: 395 |
| | | | c3 (3-alpha hydroxysteroid | | | |
| | | | dehydrogenase, type ii) | | | |
| RARB | 170 | 212414 | retinoic acid receptor, beta | SEQ ID No: 396 | SEQ ID No: 397 | SEQ ID No: 398 |
| MGLL | 171 | 21626 | monoglyceride lipase | SEQ ID No: 399 | | SEQ ID No: 400 |
| CRK | 172 | 22295 | v-crk sarcoma virus ct10 oncogene | | SEQ ID No: 401 | SEQ ID No: 402 |
| | | | homolog (avian) | | | |
| LAMA3 | 173 | 2266576 | laminin, alpha 3 | SEQ ID No: 403 | | SEQ ID No: 404 |
| ZDHHC1 | 174 | 2272404 | zinc finger, dhhc domain containing 1 | SEQ ID No: 405 | | SEQ ID No: 406 |
| BCL2 | 175 | 232714 | b-cell cll/lymphoma 2 | SEQ ID No: 407 | | SEQ ID No: 408 |
| VPREB3 | 176 | 2349125 | pre-b lymphocyte gene 3 | SEQ ID No: 409 | | SEQ ID No: 410 |
| PFC | 177 | 235934 | properdin p factor, complement | SEQ ID No: 411 | SEQ ID No: 412 | SEQ ID No: 413 |
| BAK1 | 178 | 235938 | bcl2-antagonist/killer 1 | SEQ ID No: 414 | SEQ ID No: 415 | SEQ ID No: 416 |
| MGC13071 | 179 | 236008 | hypothetical protein mgc13071 | SEQ ID No: 417 | SEQ ID No: 418 | SEQ ID No: 419 |
| TP53 | 180 | 236338 | tumor protein p53 (li-fraumeni | SEQ ID No: 420 | SEQ ID No: 421 | SEQ ID No: 298 |
| | | | syndrome) | | | |
| CAPN2 | 181 | 23643 | calpain 2, (m/ii) large subunit | SEQ ID No: 422 | SEQ ID No: 423 | SEQ ID No: 424 |
| ARAF1 | 182 | 23692 | v-raf murine sarcoma 3611 viral | SEQ ID No: 425 | SEQ ID No: 426 | SEQ ID No: 427 |
| | | | oncogene homolog 1 | | | |
| QDPR | 183 | 23776 | quinoid dihydropteridine reductase | SEQ ID No: 428 | SEQ ID No: 429 | SEQ ID No: 430 |
| SLC12A2 | 184 | 238612 | solute carrier family 12 | SEQ ID No: 431 | SEQ ID No: 432 | SEQ ID No: 433 |
| | | | (sodium/potassium/chloride | | | |
| | | | transporters), member 2 | | | |
| MGC5395 | 185 | 238840 | hypothetical protein mgc5395 | SEQ ID No: 434 | SEQ ID No: 435 | SEQ ID No: 436 |
| GCSH | 186 | 239937 | glycine cleavage system protein h | | SEQ ID No: 437 | SEQ ID No: 438 |
| | | | (aminomethyl carrier) | | | |
| EPHB2 | 187 | 24067 | ephb2 | SEQ ID No: 439 | | SEQ ID No: 440 |
| | 188 | 240753 | | SEQ ID No: 441 | SEQ ID No: 442 | |
| TPP2 | 189 | 24085 | tripeptidyl peptidase ii | SEQ ID No: 443 | SEQ ID No: 444 | SEQ ID No: 445 |
| TPP2 | 190 | 241151 | tripeptidyl peptidase ii | SEQ ID No: 446 | SEQ ID No: 447 | SEQ ID No: 445 |
| IQGAP1 | 191 | 24125 | iq motif containing gtpase activating | SEQ ID No: 448 | SEQ ID No: 449 | SEQ ID No: 450 |
| | | | protein 1 | | | |
| FGB | 192 | 241788 | fibrinogen, b beta polypeptide | SEQ ID No: 451 | SEQ ID No: 452 | SEQ ID No: 343 |
| FGA | 193 | 244810 | fibrinogen, a alpha polypeptide | SEQ ID No: 453 | | SEQ ID No: 454 |
| CTSS | 194 | 245614 | cathepsin s | SEQ ID No: 455 | SEQ ID No: 456 | SEQ ID No: 457 |
| FAM3A | 195 | 24609 | family with sequence similarity 3, | SEQ ID No: 458 | SEQ ID No: 459 | SEQ ID No: 460 |
| | | | member a | | | |
| GSN | 196 | 246170 | gelsolin (amyloidosis, finnish type) | SEQ ID No: 461 | SEQ ID No: 462 | SEQ ID No: 463 |
| IDE | 197 | 246290 | insulin-degrading enzyme | SEQ ID No: 464 | | SEQ ID No: 465 |
| ADH4 | 198 | 246860 | alcohol dehydrogenase 4 (class ii), pi | SEQ ID No: 466 | SEQ ID No: 467 | SEQ ID No: 468 |
| | | | polypeptide | | | |
| DSC2 | 199 | 247055 | desmocollin 2 | SEQ ID No: 469 | SEQ ID No: 470 | SEQ ID No: 471 |
| K-ALPHA-1 | 200 | 247905 | tubulin, alpha, ubiquitous | SEQ ID No: 472 | | SEQ ID No: 473 |
| ATP6V1H | 201 | 247909 | atpase, h+ transporting, lysosomal | SEQ ID No: 474 | | SEQ ID No: 475 |
| | | | 50/57 kda, v1 subunit h | | | |
| COX5B | 202 | 248263 | cytochrome c oxidase subunit vb | SEQ ID No: 476 | SEQ ID No: 477 | SEQ ID No: 478 |
| DLK1 | 203 | 248701 | delta-like 1 homolog (drosophila) | SEQ ID No: 479 | | SEQ ID No: 480 |
| CNTN1 | 204 | 24884 | contactin 1 | SEQ ID No: 481 | SEQ ID No: 482 | SEQ ID No: 483 |
| CDC42 | 205 | 251772 | cell division cycle 42 (gtp binding | SEQ ID No: 484 | | SEQ ID No: 485 |
| | | | protein, 25 kda) | | | |
| SCO1 | 206 | 25222 | sco cytochrome oxidase deficient | SEQ ID No: 486 | | SEQ ID No: 487 |
| | | | homolog 1 (yeast) | | | |
| LOC51058 | 207 | 25285 | hypothetical protein loc51058 | SEQ ID No: 488 | | SEQ ID No: 489 |
| RALB | 208 | 25392 | v-ral simian leukemia viral oncogene | SEQ ID No: 490 | SEQ ID No: 491 | SEQ ID No: 492 |
| | | | homolog b (ras related; gtp binding | | | |
| | | | protein) | | | |
| RPL3 | 209 | 254505 | ribosomal protein 13 | SEQ ID No: 493 | | SEQ ID No: 494 |
| SLPI | 210 | 255348 | secretory leukocyte protease inhibitor | SEQ ID No: 495 | | SEQ ID No: 496 |
| | | | (antileukoproteinase) | | | |
| HIPK3 | 211 | 256846 | homeodomain interacting protein kinase 3 | SEQ ID No: 497 | SEQ ID No: 498 | SEQ ID No: 499 |
| NIT1 | 212 | 257170 | nitrilase 1 | SEQ ID No: 500 | SEQ ID No: 501 | SEQ ID No: 502 |
| RPL39 | 213 | 257284 | ribosomal protein 139 | SEQ ID No: 503 | | SEQ ID No: 504 |
| UCHL3 | 214 | 257445 | ubiquitin carboxyl-terminal esterase 13 | SEQ ID No: 505 | SEQ ID No: 506 | SEQ ID No: 507 |
| | | | (ubiquitin thiolesterase) | | | |
| MAD | 215 | 257519 | max dimerization protein 1 | SEQ ID No: 508 | | SEQ ID No: 509 |
| DUSP1 | 216 | 257708 | dual specificity phosphatase 1 | SEQ ID No: 510 | | SEQ ID No: 511 |
| COX7B | 217 | 258313 | cytochrome c oxidase subunit viib | SEQ ID No: 512 | | SEQ ID No: 513 |
| KRT6B | 218 | 25831 | keratin 6b | SEQ ID No: 514 | SEQ ID No: 515 | SEQ ID No: 516 |
| CYP19A1 | 219 | 258870 | cytochrome p450, family 19, subfamily | SEQ ID No: 517 | SEQ ID No: 518 | SEQ ID No: 519 |
| | | | a, polypeptide 1 | | | |
| HPSE | 220 | 260138 | heparanase | SEQ ID No: 520 | SEQ ID No: 521 | SEQ ID No: 522 |
| CTCF | 221 | 26029 | ccctc-binding factor (zinc finger | SEQ ID No: 523 | SEQ ID No: 524 | SEQ ID No: 525 |
| | | | protein) | | | |
| HMGA2 | 222 | 261204 | high mobility group at-hook 2 | SEQ ID No: 526 | | SEQ ID No: 527 |
| CTSB | 223 | 261517 | cathepsin b | SEQ ID No: 528 | | SEQ ID No: 529 |
| GK | 224 | 262425 | glycerol kinase | | SEQ ID No: 530 | SEQ ID No: 531 |
| IL6ST | 225 | 263262 | interleukin 6 signal transducer (gp 130, | SEQ ID No: 532 | | SEQ ID No: 533 |
| | | | oncostatin m receptor) | | | |
| C5ORF5 | 226 | 264183 | chromosome 5 open reading frame 5 | SEQ ID No: 534 | SEQ ID No: 535 | SEQ ID No: 536 |
| LOC57209 | 227 | 264186 | kruppel-type zinc finger protein | SEQ ID No: 537 | SEQ ID No: 538 | |
| CRYAB | 228 | 264331 | crystallin, alpha b | SEQ ID No: 539 | SEQ ID No: 540 | SEQ ID No: 541 |
| MGC9850 | 229 | 26584 | hypothetical protein mgc9850 | SEQ ID No: 542 | | SEQ ID No: 543 |
| CCT4 | 230 | 26710 | chaperonin containing tcpl, subunit 4 | SEQ ID No: 544 | SEQ ID No: 545 | SEQ ID No: 546 |
| | | | (delta) | | | |
| LIAS | 231 | 267123 | lipoic acid synthetase | SEQ ID No: 547 | SEQ ID No: 548 | SEQ ID No: 549 |
| HMGB2 | 232 | 267145 | high-mobility group box 2 | SEQ ID No: 550 | SEQ ID No: 551 | SEQ ID No: 552 |
| MAGEH1 | 233 | 267657 | apr-1 protein | SEQ ID No: 553 | SEQ ID No: 554 | SEQ ID No: 555 |
| MADH1 | 234 | 268150 | mad, mothers against decapentaplegic | SEQ ID No: 556 | SEQ ID No: 557 | SEQ ID No: 558 |
| | | | homolog 1 (drosophila) | | | |
| ACADVL | 235 | 269388 | acyl-coenzyme a dehydrogenase, very | SEQ ID No: 559 | | SEQ ID No: 560 |
| | | | long chain | | | |
| RENT1 | 236 | 26945 | regulator of nonsense transcripts 1 | SEQ ID No: 561 | SEQ ID No: 562 | SEQ ID No: 563 |
| PWP1 | 237 | 26964 | nuclear phosphoprotein similar to | SEQ ID No: 564 | SEQ ID No: 565 | SEQ ID No: 566 |
| | | | s. cerevisiae pwp1 | | | |
| PTD004 | 238 | 270794 | hypothetical protein ptd004 | SEQ ID No: 567 | SEQ ID No: 568 | SEQ ID No: 569 |
| | 239 | 27100 | | SEQ ID No: 570 | SEQ ID No: 571 | |
| ASNS | 240 | 27208 | asparagine synthetase | SEQ ID No: 572 | SEQ ID No: 573 | SEQ ID No: 574 |
| NRAS | 241 | 272189 | neuroblastoma ras viral (v-ras) | SEQ ID No: 575 | SEQ ID No: 576 | SEQ ID No: 577 |
| | | | oncogene homolog | | | |
| MORF4L1 | 242 | 27237 | mortality factor 4 like 1 | SEQ ID No: 578 | | SEQ ID No: 579 |
| CCT4 | 243 | 272502 | chaperonin containing tcp1, subunit 4 | SEQ ID No: 580 | | SEQ ID No: 546 |
| | | | (delta) | | | |
| WBSCR22 | 244 | 27326 | williams beuren syndrome chromosome | SEQ ID No: 581 | SEQ ID No: 582 | SEQ ID No: 583 |
| | | | region 22 | | | |
| GNS | 245 | 274315 | glucosamine (n-acetyl)-6-sulfatase | SEQ ID No: 584 | SEQ ID No: 585 | SEQ ID No: 586 |
| | | | (sanfilippo disease iiid) | | | |
| SLC17A7 | 246 | 27506 | solute carrier family 17 (sodium- | SEQ ID No: 587 | | SEQ ID No: 588 |
| | | | dependent inorganic phosphate | | | |
| | | | cotransporter), member 7 | | | |
| ARHT2 | 247 | 27599 | ras homolog gene family, member t2 | SEQ ID No: 589 | SEQ ID No: 590 | SEQ ID No: 591 |
| TP53BP2 | 248 | 277339 | tumor protein p53 binding protein, 2 | SEQ ID No: 592 | SEQ ID No: 593 | SEQ ID No: 594 |
| CCBL1 | 249 | 277740 | cysteine conjugate-beta lyase; | SEQ ID No: 595 | SEQ ID No: 596 | SEQ ID No: 597 |
| | | | cytoplasmic (glutamine transaminase k, | | | |
| | | | kyneurenine aminotransferase) | | | |
| ID4 | 250 | 2783684 | inhibitor of dna binding 4, dominant | SEQ ID No: 598 | SEQ ID No: 599 | SEQ ID No: 600 |
| | | | negative helix-loop-helix protein | | | |
| TUBE1 | 251 | 279460 | tubulin, epsilon 1 | SEQ ID No: 601 | SEQ ID No: 602 | SEQ ID No: 603 |
| MPDZ | 252 | 28019 | multiple pdz domain protein | SEQ ID No: 604 | SEQ ID No: 605 | SEQ ID No: 606 |
| CACNA1I | 253 | 283375 | calcium channel, voltage-dependent, | SEQ ID No: 607 | SEQ ID No: 608 | SEQ ID No: 609 |
| | | | alpha 1i subunit | | | |
| GFER | 254 | 283601 | growth factor, augmenter of liver | SEQ ID No: 610 | SEQ ID No: 611 | SEQ ID No: 612 |
| | | | regeneration (erv1 homolog, s. cerevisiae | | | |
| SNRPB2 | 255 | 284256 | small nuclear ribonucleoprotein | SEQ ID No: 613 | | SEQ ID No: 614 |
| | | | polypeptide b″ | | | |
| CHI3L2 | 256 | 284640 | chitinase 3-like 2 | SEQ ID No: 615 | | SEQ ID No: 616 |
| ABCA8 | 257 | 284828 | atp-binding cassette, sub-family a | SEQ ID No: 617 | | SEQ ID No: 618 |
| | | | (abc1), member 8 | | | |
| BTBD1 | 258 | 28577 | btb (poz) domain containing 1 | SEQ ID No: 619 | SEQ ID No: 620 | SEQ ID No: 621 |
| MMP13 | 259 | 285780 | matrix metalloproteinase 13 | SEQ ID No: 622 | | SEQ ID No: 623 |
| | | | (collagenase 3) | | | |
| GART | 260 | 28596 | phosphoribosylglycinamide | SEQ ID No: 624 | SEQ ID No: 625 | SEQ ID No: 626 |
| | | | formyltransferase, | | | |
| | | | phosphoribosylglycinamide synthetase, | | | |
| | | | phosphoribosylaminoimidazole | | | |
| | | | synthetase | | | |
| CUL2 | 261 | 286287 | cullin 2 | SEQ ID No: 627 | | SEQ ID No: 628 |
| GRM3 | 262 | 287843 | glutamate receptor, metabotropic 3 | SEQ ID No: 629 | | SEQ ID No: 630 |
| CA7 | 263 | 288874 | carbonic anhydrase vii | SEQ ID No: 631 | SEQ ID No: 632 | SEQ ID No: 633 |
| PNMT | 264 | 289857 | phenylethanolamine n- | SEQ ID No: 634 | | SEQ ID No: 635 |
| | | | methyltransferase | | | |
| SILV | 265 | 291448 | silver homolog (mouse) | SEQ ID No: 636 | SEQ ID No: 637 | SEQ ID No: 638 |
| ANK1 | 266 | 292321 | ankyrin 1, erythrocytic | SEQ ID No: 639 | SEQ ID No: 640 | SEQ ID No: 641 |
| XRCC1 | 267 | 29451 | x-ray repair complementing defective | SEQ ID No: 642 | SEQ ID No: 643 | SEQ ID No: 644 |
| | | | repair in chinese hamster cells 1 | | | |
| CSE1L | 268 | 29933 | cse1 chromosome segregation 1-like | SEQ ID No: 645 | SEQ ID No: 646 | SEQ ID No: 647 |
| | | | (yeast) | | | |
| DXS1283E | 269 | 300163 | gs2 gene | SEQ ID No: 648 | SEQ ID No: 649 | |
| TAF10 | 270 | 30066 | taf10 rna polymerase ii, tata box | SEQ ID No: 650 | | SEQ ID No: 651 |
| | | | binding protein (tbp)-associated factor, | | | |
| | | | 30 kda | | | |
| CKMT2 | 271 | 301119 | creatine kinase, mitochondrial 2 | SEQ ID No: 652 | SEQ ID No: 653 | SEQ ID No: 654 |
| | | | (sarcomeric) | | | |
| TNNC1 | 272 | 301128 | troponin c, slow | SEQ ID No: 655 | | SEQ ID No: 656 |
| DKFZP434J0617 | 273 | 301258 | hypothetical protein dkfzp434j0617 | SEQ ID No: 657 | | |
| | 274 | 302310 | homo sapiens cdna flj36340 fis, clone | SEQ ID No: 658 | SEQ ID No: 659 | |
| | | | thymu2006468. | | | |
| GUK1 | 275 | 302453 | guanylate kinase 1 | | SEQ ID No: 660 | SEQ ID No: 661 |
| HSPA9B | 276 | 305045 | heat shock 70 kda protein 9b (mortalin- | SEQ ID No: 662 | SEQ ID No: 663 | SEQ ID No: 664 |
| | | | 2) | | | |
| NDUFA6 | 277 | 306510 | nadh dehydrogenase (ubiquinone) 1 | SEQ ID No: 665 | SEQ ID No: 666 | SEQ ID No: 667 |
| | | | alpha subcomplex, 6, 14 kda | | | |
| IFNGR2 | 278 | 306555 | interferon gamma receptor 2 (interferon | SEQ ID No: 668 | SEQ ID No: 669 | SEQ ID No: 670 |
| | | | gamma transducer 1) | | | |
| HRIHFB2206 | 279 | 306697 | hrihfb2206 protein | SEQ ID No: 671 | SEQ ID No: 672 | |
| GCAT | 280 | 307094 | glycine c-acetyltransferase (2-amino-3- | SEQ ID No: 673 | SEQ ID No: 674 | SEQ ID No: 675 |
| | | | ketobutyrate coenzyme a ligase) | | | |
| CD9 | 281 | 307352 | cd9 antigen (p24) | SEQ ID No: 676 | SEQ ID No: 677 | SEQ ID No: 678 |
| ESD | 282 | 310057 | esterase d/formylglutathione hydrolase | SEQ ID No: 679 | SEQ ID No: 680 | |
| ZNF183 | 283 | 310088 | zinc finger protein 183 (ring finger, | SEQ ID No: 681 | SEQ ID No: 682 | SEQ ID No: 683 |
| | | | c3hc4 type) | | | |
| HSPA8 | 284 | 31027 | heat shock 70 kda protein 8 | SEQ ID No: 684 | SEQ ID No: 685 | SEQ ID No: 686 |
| RPL35 | 285 | 310774 | ribosomal protein 135 | SEQ ID No: 687 | SEQ ID No: 688 | SEQ ID No: 689 |
| NUDT5 | 286 | 310860 | nudix (nucleoside diphosphate linked | SEQ ID No: 690 | SEQ ID No: 691 | SEQ ID No: 692 |
| | | | moiety x)-type motif 5 | | | |
| PFDN4 | 287 | 320143 | prefoldin 4 | SEQ ID No: 693 | SEQ ID No: 694 | SEQ ID No: 695 |
| RPL37 | 288 | 320151 | ribosomal protein 137 | SEQ ID No: 696 | SEQ ID No: 697 | SEQ ID No: 698 |
| SPR | 289 | 320457 | sepiapterin reductase (7,8- | SEQ ID No: 699 | SEQ ID No: 700 | SEQ ID No: 701 |
| | | | dihydrobiopterin:nadp + | | | |
| | | | oxidoreductase) | | | |
| LOC56267 | 290 | 320775 | hypothetical protein 669 | SEQ ID No: 702 | SEQ ID No: 703 | SEQ ID No: 704 |
| RPL31 | 291 | 321259 | ribosomal protein 131 | SEQ ID No: 705 | SEQ ID No: 706 | SEQ ID No: 707 |
| SRP72 | 292 | 321510 | signal recognition particle 72 kda | SEQ ID No: 708 | SEQ ID No: 709 | SEQ ID No: 710 |
| RPS6 | 293 | 321733 | ribosomal protein s6 | SEQ ID No: 711 | SEQ ID No: 712 | SEQ ID No: 713 |
| PHKG1 | 294 | 321783 | phosphorylase kinase, gamma 1 | SEQ ID No: 714 | SEQ ID No: 715 | SEQ ID No: 716 |
| | | | (muscle) | | | |
| TACSTD1 | 295 | 321907 | tumor-associated calcium signal | SEQ ID No: 717 | SEQ ID No: 718 | SEQ ID No: 719 |
| | | | transducer 1 | | | |
| RPS27L | 296 | 321973 | ribosomal protein s27-like | SEQ ID No: 720 | SEQ ID No: 721 | SEQ ID No: 722 |
| | 297 | 321981 | loc151103 | SEQ ID No: 723 | SEQ ID No: 724 | |
| CHGA | 298 | 322452 | chromogranin a (parathyroid secretory | SEQ ID No: 725 | SEQ ID No: 726 | SEQ ID No: 727 |
| | | | protein 1) | | | |
| SNRPC | 299 | 322471 | small nuclear ribonucleoprotein | SEQ ID No: 728 | SEQ ID No: 729 | SEQ ID No: 730 |
| | | | polypeptide c | | | |
| AIP | 300 | 322495 | aryl hydrocarbon receptor interacting | SEQ ID No: 731 | SEQ ID No: 732 | SEQ ID No: 733 |
| | | | protein | | | |
| IRF1 | 301 | 323001 | interferon regulatory factor 1 | SEQ ID No: 734 | SEQ ID No: 735 | SEQ ID No: 736 |
| COX7A2 | 302 | 323650 | cytochrome c oxidase subunit viia | SEQ ID No: 737 | SEQ ID No: 738 | SEQ ID No: 739 |
| | | | polypeptide 2 (liver) | | | |
| LOC51255 | 303 | 323681 | hypothetical protein loc51255 | SEQ ID No: 740 | SEQ ID No: 741 | SEQ ID No: 742 |
| COPZ2 | 304 | 323753 | coatomer protein complex, subunit zeta 2 | SEQ ID No: 743 | SEQ ID No: 744 | SEQ ID No: 745 |
| CKAP1 | 305 | 323766 | cytoskeleton-associated protein 1 | SEQ ID No: 746 | | SEQ ID No: 747 |
| RPS3A | 306 | 323863 | ribosomal protein s3a | SEQ ID No: 748 | SEQ ID No: 749 | SEQ ID No: 750 |
| SOX9 | 307 | 323948 | sry (sex determining region y)-box 9 | SEQ ID No: 751 | | SEQ ID No: 752 |
| | | | (campomelic dysplasia, autosomal sex- | | | |
| | | | reversal) | | | |
| DSCR1 | 308 | 324006 | down syndrome critical region gene 1 | SEQ ID No: 753 | SEQ ID No: 754 | SEQ ID No: 755 |
| KRAS2 | 309 | 324257 | v-ki-ras2 kirsten rat sarcoma 2 viral | SEQ ID No: 756 | SEQ ID No: 757 | SEQ ID No: 758 |
| | | | oncogene homolog | | | |
| CTBS | 310 | 324369 | chitobiase, di-n-acetyl- | SEQ ID No: 759 | | SEQ ID No: 760 |
| PPP1R15A | 311 | 324684 | protein phosphatase 1, regulatory | SEQ ID No: 761 | SEQ ID No: 762 | SEQ ID No: 763 |
| | | | (inhibitor) subunit 15a | | | |
| RPS15A | 312 | 324757 | ribosomal protein s15a | SEQ ID No: 764 | SEQ ID No: 765 | SEQ ID No: 311 |
| SAT | 313 | 324930 | spermidine/spermine n1- | SEQ ID No: 766 | SEQ ID No: 767 | SEQ ID No: 768 |
| | | | acetyltransferase | | | |
| GRSF1 | 314 | 325058 | g-rich rna sequence binding factor 1 | SEQ ID No: 769 | SEQ ID No: 770 | SEQ ID No: 771 |
| PSG5 | 315 | 325641 | pregnancy specific beta-1-glycoprotein 5 | SEQ ID No: 772 | SEQ ID No: 773 | SEQ ID No: 774 |
| STMN4 | 316 | 32698 | stathmin-like 4 | SEQ ID No: 775 | SEQ ID No: 776 | SEQ ID No: 777 |
| CDH15 | 317 | 327684 | cadherin 15, m-cadherin (myotubule) | SEQ ID No: 778 | SEQ ID No: 779 | SEQ ID No: 780 |
| NDUFA4 | 318 | 327740 | nadh dehydrogenase (ubiquinone) 1 | SEQ ID No: 781 | SEQ ID No: 782 | SEQ ID No: 320 |
| | | | alpha subcomplex, 4, 9 kda | | | |
| RAN | 319 | 328245 | ran, member ras oncogene family | SEQ ID No: 783 | SEQ ID No: 784 | SEQ ID No: 785 |
| PNLIPRP1 | 320 | 328591 | pancreatic lipase-related protein 1 | SEQ ID No: 786 | SEQ ID No: 787 | SEQ ID No: 788 |
| CAP2 | 321 | 33005 | cap, adenylate cyclase-associated | SEQ ID No: 789 | SEQ ID No: 790 | SEQ ID No: 791 |
| | | | protein, 2 (yeast) | | | |
| NDFIP2 | 322 | 33722 | nedd4 family interacting protein 2 | | SEQ ID No: 792 | |
| ATP5C1 | 323 | 33794 | atp synthase, h+ transporting, | SEQ ID No: 793 | SEQ ID No: 794 | SEQ ID No: 109 |
| | | | mitochondrial f1 complex, gamma | | | |
| | | | polypeptide 1 | | | |
| ATP7A | 324 | 340995 | atpase, cu++ transporting, alpha | SEQ ID No: 795 | SEQ ID No: 796 | SEQ ID No: 797 |
| | | | polypeptide (menkes syndrome) | | | |
| ATP6V0B | 325 | 341121 | atpase, h+ transporting, lysosomal | SEQ ID No: 798 | SEQ ID No: 799 | SEQ ID No: 800 |
| | | | 21 kda, v0 subunit c″ | | | |
| DAD1 | 326 | 341699 | defender against cell death 1 | SEQ ID No: 801 | SEQ ID No: 802 | SEQ ID No: 803 |
| | 327 | 341834 | loc349507 | SEQ ID No: 804 | SEQ ID No: 805 | |
| | 328 | 341984 | | SEQ ID No: 806 | SEQ ID No: 807 | |
| CXORF6 | 329 | 342054 | chromosome x open reading frame 6 | SEQ ID No: 808 | SEQ ID No: 809 | SEQ ID No: 810 |
| B2M | 330 | 342416 | beta-2-microglobulin | SEQ ID No: 811 | SEQ ID No: 812 | SEQ ID No: 813 |
| CLIC5 | 331 | 34260 | chloride intracellular channel 5 | SEQ ID No: 814 | SEQ ID No: 815 | SEQ ID No: 816 |
| NDN | 332 | 343578 | necdin homolog (mouse) | SEQ ID No: 817 | SEQ ID No: 818 | SEQ ID No: 819 |
| OSBPL1A | 333 | 344037 | oxysterol binding protein-like 1a | SEQ ID No: 820 | SEQ ID No: 821 | SEQ ID No: 822 |
| COL6A1 | 334 | 344326 | collagen, type vi, alpha 1 | SEQ ID No: 823 | SEQ ID No: 824 | SEQ ID No: 825 |
| MRPS23 | 335 | 344792 | mitochondrial ribosomal protein s23 | SEQ ID No: 826 | SEQ ID No: 827 | SEQ ID No: 828 |
| PIK3CA | 336 | 345430 | phosphoinositide-3-kinase, catalytic, | SEQ ID No: 829 | SEQ ID No: 830 | SEQ ID No: 831 |
| | | | alpha polypeptide | | | |
| C6ORF9 | 337 | 345437 | chromosome 6 open reading frame 9 | SEQ ID No: 832 | SEQ ID No: 833 | SEQ ID No: 834 |
| FLJ20813 | 338 | 345648 | hypothetical protein flj20813 | SEQ ID No: 835 | SEQ ID No: 836 | SEQ ID No: 837 |
| RPS21 | 339 | 345676 | ribosomal protein s21 | SEQ ID No: 838 | SEQ ID No: 839 | SEQ ID No: 840 |
| | 340 | 345694 | | SEQ ID No: 841 | SEQ ID No: 842 | |
| CA3 | 341 | 345706 | carbonic anhydrase iii, muscle specific | SEQ ID No: 843 | SEQ ID No: 844 | SEQ ID No: 845 |
| P4HA1 | 342 | 346016 | procollagen-proline, 2-oxoglutarate 4- | SEQ ID No: 846 | SEQ ID No: 847 | SEQ ID No: 848 |
| | | | dioxygenase (proline 4-hydroxylase), | | | |
| | | | alpha polypeptide i | | | |
| COL6A2 | 343 | 346269 | collagen, type vi, alpha 2 | SEQ ID No: 849 | SEQ ID No: 850 | SEQ ID No: 851 |
| SFN | 344 | 346610 | Stratifin | SEQ ID No: 852 | SEQ ID No: 853 | SEQ ID No: 854 |
| TCEB1 | 345 | 347373 | transcription elongation factor b (siii), | SEQ ID No: 855 | SEQ ID No: 856 | SEQ ID No: 857 |
| | | | polypeptide 1 (15 kda, elongin c) | | | |
| RELN | 346 | 34888 | Reelin | SEQ ID No: 858 | SEQ ID No: 859 | SEQ ID No: 860 |
| SKP1A | 347 | 34917 | s-phase kinase-associated protein 1a | SEQ ID No: 861 | SEQ ID No: 862 | SEQ ID No: 863 |
| | | | (p19a) | | | |
| AQP1 | 348 | 35072 | aquaporin 1 (channel-forming integral | SEQ ID No: 864 | SEQ ID No: 865 | SEQ ID No: 866 |
| | | | protein, 28 kda) | | | |
| IRF2 | 349 | 35262 | interferon regulatory factor 2 | SEQ ID No: 867 | SEQ ID No: 868 | SEQ ID No: 869 |
| NGB | 350 | 35483 | Neuroglobin | SEQ ID No: 870 | SEQ ID No: 871 | SEQ ID No: 872 |
| TM4SF5 | 351 | 356783 | transmembrane 4 superfamily member 5 | SEQ ID No: 873 | SEQ ID No: 874 | SEQ ID No: 875 |
| TGFB3 | 352 | 356980 | transforming growth factor, beta 3 | SEQ ID No: 876 | SEQ ID No: 877 | SEQ ID No: 878 |
| RPA3 | 353 | 357239 | replication protein a3, 14 kda | SEQ ID No: 879 | SEQ ID No: 880 | SEQ ID No: 881 |
| SEMA3C | 354 | 357820 | sema domain, immunoglobulin domain | SEQ ID No: 882 | SEQ ID No: 883 | SEQ ID No: 884 |
| | | | (ig), short basic domain, secreted, | | | |
| | | | (semaphorin) 3c | | | |
| CNOT2 | 355 | 357893 | ccr4-not transcription complex, subunit 2 | SEQ ID No: 885 | | SEQ ID No: 886 |
| CDW52 | 356 | 358041 | cdw52 antigen (campath-1 antigen) | SEQ ID No: 887 | SEQ ID No: 888 | SEQ ID No: 889 |
| SOX9 | 357 | 358117 | sry (sex determining region y)-box 9 | SEQ ID No: 890 | SEQ ID No: 891 | SEQ ID No: 752 |
| | | | (campomelic dysplasia, autosomal sex- | | | |
| | | | reversal) | | | |
| HSU79266 | 358 | 358162 | protein predicted by clone 23627 | SEQ ID No: 892 | SEQ ID No: 893 | SEQ ID No: 894 |
| PFDN2 | 359 | 358267 | prefoldin 2 | SEQ ID No: 895 | SEQ ID No: 896 | SEQ ID No: 897 |
| TPM1 | 360 | 358683 | tropomyosin 1 (alpha) | SEQ ID No: 898 | SEQ ID No: 899 | SEQ ID No: 900 |
| FLJ21272 | 361 | 358943 | hypothetical protein flj21272 | SEQ ID No: 901 | SEQ ID No: 902 | SEQ ID No: 903 |
| PSMC2 | 362 | 358993 | proteasome (prosome, macropain) 26s | SEQ ID No: 904 | | SEQ ID No: 905 |
| | | | subunit, atpase, 2 | | | |
| CKS2 | 363 | 359119 | cdc28 protein kinase regulatory subunit 2 | SEQ ID No: 906 | | SEQ ID No: 907 |
| NDUFA9 | 364 | 359147 | nadh dehydrogenase (ubiquinone) 1 | SEQ ID No: 908 | | SEQ ID No: 909 |
| | | | alpha subcomplex, 9, 39 kda | | | |
| H11 | 365 | 359191 | protein kinase h11 | SEQ ID No: 910 | | SEQ ID No: 911 |
| CA4 | 366 | 359250 | carbonic anhydrase iv | SEQ ID No: 912 | SEQ ID No: 913 | SEQ ID No: 914 |
| PRSS3 | 367 | 359254 | protease, serine, 3 (mesotrypsin) | SEQ ID No: 915 | SEQ ID No: 916 | SEQ ID No: 917 |
| | 368 | 360588 | homo sapiens transcribed sequence with | SEQ ID No: 918 | | |
| | | | moderate similarity to protein | | | |
| | | | ref: np_036199.1 (h. sapiens) aldo-keto | | | |
| | | | reductase family 7, member a3 | | | |
| | | | (aflatoxin aldehyde reductase) [homo | | | |
| | | | sapiens] | | | |
| HIG1 | 369 | 361108 | likely ortholog of mouse hypoxia | SEQ ID No: 919 | SEQ ID No: 920 | SEQ ID No: 921 |
| | | | induced gene 1 | | | |
| | 370 | 363273 | | SEQ ID No: 922 | SEQ ID No: 923 | |
| ADD1 | 371 | 363991 | adducin 1 (alpha) | SEQ ID No: 924 | SEQ ID No: 925 | SEQ ID No: 68 |
| LAMB1 | 372 | 364012 | laminin, beta 1 | SEQ ID No: 926 | SEQ ID No: 927 | SEQ ID No: 928 |
| CD5 | 373 | 364687 | cd5 antigen (p56-62) | SEQ ID No: 929 | SEQ ID No: 930 | SEQ ID No: 931 |
| UQCR | 374 | 36607 | ubiquinol-cytochrome c reductase | SEQ ID No: 932 | SEQ ID No: 933 | SEQ ID No: 934 |
| | | | (6.4 kd) subunit | | | |
| RAP2A | 375 | 36684 | rap2a, member of ras oncogene family | SEQ ID No: 935 | SEQ ID No: 936 | SEQ ID No: 937 |
| RGS6 | 376 | 36710 | regulator of g-protein signalling 6 | SEQ ID No: 938 | SEQ ID No: 939 | SEQ ID No: 940 |
| IL1RN | 377 | 36844 | interleukin 1 receptor antagonist | SEQ ID No: 941 | SEQ ID No: 942 | SEQ ID No: 943 |
| LRP1 | 378 | 37345 | low density lipoprotein-related protein | SEQ ID No: 944 | SEQ ID No: 945 | SEQ ID No: 946 |
| | | | 1 (alpha-2-macroglobulin receptor) | | | |
| DJ1042K10.2 | 379 | 37496 | hypothetical protein dj1042k10.2 | SEQ ID No: 947 | SEQ ID No: 948 | SEQ ID No: 949 |
| PTPRN2 | 380 | 37506 | protein tyrosine phosphatase, receptor | SEQ ID No: 950 | SEQ ID No: 951 | SEQ ID No: 952 |
| | | | type, n polypeptide 2 | | | |
| CCNB2 | 381 | 375781 | cyclin b2 | SEQ ID No: 953 | SEQ ID No: 954 | SEQ ID No: 955 |
| TCTEL1 | 382 | 376284 | t-complex-associated-testis-expressed | SEQ ID No: 956 | SEQ ID No: 957 | SEQ ID No: 958 |
| | | | 1-like 1 | | | |
| TUBB | 383 | 37630 | tubulin, beta polypeptide | SEQ ID No: 959 | | SEQ ID No: 960 |
| RHEB | 384 | 376473 | ras homolog enriched in brain | SEQ ID No: 961 | SEQ ID No: 962 | SEQ ID No: 963 |
| VCP | 385 | 376547 | valosin-containing protein | SEQ ID No: 964 | | SEQ ID No: 965 |
| IL2RB | 386 | 376696 | interleukin 2 receptor, beta | SEQ ID No: 966 | SEQ ID No: 967 | SEQ ID No: 152 |
| TAZ | 387 | 376755 | transcriptional co-activator with pdz- | SEQ ID No: 968 | SEQ ID No: 969 | SEQ ID No: 970 |
| | | | binding motif (taz) | | | |
| HSPC150 | 388 | 376769 | hspc150 protein similar to ubiquitin- | SEQ ID No: 971 | SEQ ID No: 972 | SEQ ID No: 973 |
| | | | conjugating enzyme | | | |
| PLCD4 | 389 | 376802 | phospholipase c, delta 4 | SEQ ID No: 974 | SEQ ID No: 975 | SEQ ID No: 976 |
| NR2F6 | 390 | 377020 | nuclear receptor subfamily 2, group f, | | SEQ ID No: 977 | SEQ ID No: 978 |
| | | | member 6 | | | |
| MTPN | 391 | 377545 | Myotrophin | SEQ ID No: 979 | | SEQ ID No: 980 |
| SLPI | 392 | 378813 | secretory leukocyte protease inhibitor | SEQ ID No: 981 | | SEQ ID No: 496 |
| | | | (antileukoproteinase) | | | |
| KPNA1 | 393 | 38056 | karyopherin alpha 1 (importin alpha 5) | SEQ ID No: 982 | SEQ ID No: 983 | SEQ ID No: 984 |
| LAMR1 | 394 | 383433 | laminin receptor 1 (ribosomal protein | SEQ ID No: 985 | SEQ ID No: 986 | SEQ ID No: 987 |
| | | | sa, 67 kda) | | | |
| SST | 395 | 39593 | Somatostatin | SEQ ID No: 988 | | SEQ ID No: 989 |
| ABCA5 | 396 | 39821 | atp-binding cassette, sub-family a | SEQ ID No: 990 | SEQ ID No: 991 | SEQ ID No: 992 |
| | | | (abc1), member 5 | | | |
| NME1 | 397 | 39961 | non-metastatic cells 1, protein (nm23a) | SEQ ID No: 993 | SEQ ID No: 994 | SEQ ID No: 288 |
| | | | expressed in | | | |
| ADAM23 | 398 | 39972 | a disintegrin and metalloproteinase | SEQ ID No: 995 | SEQ ID No: 996 | SEQ ID No: 997 |
| | | | domain 23 | | | |
| CYCS | 399 | 40017 | cytochrome c, somatic | SEQ ID No: 998 | SEQ ID No: 999 | SEQ ID No: 1000 |
| GCNIL1 | 400 | 40567 | gcn1 general control of amino-acid | SEQ ID No: 1001 | SEQ ID No: 1002 | |
| | | | synthesis 1-like 1 (yeast) | | | |
| RBBP1 | 401 | 40721 | retinoblastoma binding protein 1 | SEQ ID No: 1003 | SEQ ID No: 1004 | SEQ ID No: 1005 |
| CNN3 | 402 | 41099 | calponin 3, acidic | SEQ ID No: 1006 | SEQ ID No: 1007 | SEQ ID No: 1008 |
| RPL24 | 403 | 41411 | ribosomal protein 124 | SEQ ID No: 1009 | SEQ ID No: 1010 | SEQ ID No: 1011 |
| SAT | 404 | 41452 | spermidine/spermine n1- | SEQ ID No: 1012 | SEQ ID No: 1013 | SEQ ID No: 768 |
| | | | acetyltransferase | | | |
| SNRPE | 405 | 415389 | small nuclear ribonucleoprotein | SEQ ID No: 1014 | SEQ ID No: 1015 | SEQ ID No: 1016 |
| | | | polypeptide e | | | |
| ARG1 | 406 | 416060 | arginase, liver | SEQ ID No: 1017 | SEQ ID No: 1018 | SEQ ID No: 1019 |
| IL13RA2 | 407 | 41648 | interleukin 13 receptor, alpha 2 | SEQ ID No: 1020 | SEQ ID No: 1021 | SEQ ID No: 1022 |
| TXN | 408 | 416946 | Thioredoxin | SEQ ID No: 1023 | SEQ ID No: 1024 | SEQ ID No: 1025 |
| TFR2 | 409 | 417861 | transferrin receptor 2 | SEQ ID No: 1026 | SEQ ID No: 1027 | SEQ ID No: 1028 |
| NUTF2 | 410 | 41857 | nuclear transport factor 2 | SEQ ID No: 1029 | | SEQ ID No: 1030 |
| P2RX4 | 411 | 42118 | purinergic receptor p2x, ligand-gated | SEQ ID No: 1031 | SEQ ID No: 1032 | SEQ ID No: 1033 |
| | | | ion channel, 4 | | | |
| SYK | 412 | 42214 | spleen tyrosine kinase | SEQ ID No: 1034 | SEQ ID No: 1035 | SEQ ID No: 1036 |
| GPC6 | 413 | 427858 | glypican 6 | SEQ ID No: 1037 | SEQ ID No: 1038 | SEQ ID No: 1039 |
| CD1C | 414 | 428103 | cd1c antigen, c polypeptide | SEQ ID No: 1040 | SEQ ID No: 1041 | SEQ ID No: 1042 |
| CYCS | 415 | 429544 | cytochrome c, somatic | SEQ ID No: 1043 | SEQ ID No: 1044 | SEQ ID No: 1000 |
| TNFRSF7 | 416 | 430090 | tumor necrosis factor receptor | SEQ ID No: 1045 | SEQ ID No: 1046 | SEQ ID No: 1047 |
| | | | superfamily, member 7 | | | |
| | 417 | 43207 | homo sapiens transcribed sequence with | SEQ ID No: 1048 | SEQ ID No: 1049 | |
| | | | strong similarity to protein sp: o00451 | | | |
| | | | (h. sapiens) nrtr_human neurturin | | | |
| | | | receptor alpha precursor (ntnr-alpha) | | | |
| | | | (nrtnr-alpha) (tgf-beta related | | | |
| | | | neurotrophic factor receptor 2) (gdnf | | | |
| | | | receptor beta) (gdnfr-beta) (ret ligand 2) | | | |
| | | | (gfr-alpha 2) | | | |
| GALNACT-2 | 418 | 43276 | chondroitin sulfate galnact-2 | SEQ ID No: 1050 | | SEQ ID No: 1051 |
| F5 | 419 | 433155 | coagulation factor v (proaccelerin, | SEQ ID No: 1052 | | SEQ ID No: 1053 |
| | | | labile factor) | | | |
| | 420 | 43338 | homo sapiens transcribed sequence with | SEQ ID No: 1054 | | |
| | | | moderate similarity to protein | | | |
| | | | ref: np_004491.1 (h. sapiens) | | | |
| | | | heterogeneous nuclear | | | |
| | | | ribonucleoprotein c, isoform b; nuclear | | | |
| | | | ribonucleoprotein particle c1 protein; | | | |
| | | | nuclear ribonucleoprotein particle c2 | | | |
| | | | protein [homo sapiens] | | | |
| RPL15 | 421 | 43442 | ribosomal protein 115 | SEQ ID No: 1055 | | SEQ ID No: 1056 |
| RPS28 | 422 | 43493 | ribosomal protein s28 | SEQ ID No: 1057 | SEQ ID No: 1058 | SEQ ID No: 1059 |
| LDHA | 423 | 43550 | lactate dehydrogenase a | SEQ ID No: 1060 | | SEQ ID No: 1061 |
| RAN | 424 | 43638 | ran, member ras oncogene family | SEQ ID No: 1062 | SEQ ID No: 1063 | SEQ ID No: 785 |
| PPP2CA | 425 | 43760 | protein phosphatase 2 (formerly 2a), | SEQ ID No: 1064 | SEQ ID No: 1065 | SEQ ID No: 1066 |
| | | | catalytic subunit, alpha isoform | | | |
| CSNK2A1 | 426 | 43941 | casein kinase 2, alpha 1 polypeptide | SEQ ID No: 1067 | SEQ ID No: 1068 | SEQ ID No: 1069 |
| CCT3 | 427 | 44152 | chaperonin containing tcp1, subunit 3 | SEQ ID No: 1070 | SEQ ID No: 1071 | SEQ ID No: 1072 |
| | | | (gamma) | | | |
| LOC115286 | 428 | 45021 | hypothetical protein loc115286 | SEQ ID No: 1073 | SEQ ID No: 1074 | SEQ ID No: 1075 |
| SNCA | 429 | 45086 | synuclein, alpha (non a4 component of | SEQ ID No: 1076 | SEQ ID No: 1077 | SEQ ID No: 1078 |
| | | | amyloid precursor) | | | |
| MORF4L2 | 430 | 45706 | mortality factor 4 like 2 | SEQ ID No: 1079 | | SEQ ID No: 1080 |
| YWHAB | 431 | 45831 | tyrosine 3-monooxygenase/tryptophan | SEQ ID No: 1081 | SEQ ID No: 1082 | SEQ ID No: 1083 |
| | | | 5-monooxygenase activation protein, | | | |
| | | | beta polypeptide | | | |
| PCSK7 | 432 | 45900 | proprotein convertase subtilisin/kexin | SEQ ID No: 1084 | | SEQ ID No: 1085 |
| | | | type 7 | | | |
| COX7A2L | 433 | 46147 | cytochrome c oxidase subunit viia | SEQ ID No: 1086 | SEQ ID No: 1087 | SEQ ID No: 117 |
| | | | polypeptide 2 like | | | |
| DTNA | 434 | 46518 | dystrobrevin, alpha | SEQ ID No: 1088 | SEQ ID No: 1089 | SEQ ID No: 1090 |
| PPP1R7 | 435 | 46888 | protein phosphatase 1, regulatory | SEQ ID No: 1091 | SEQ ID No: 1092 | SEQ ID No: 1093 |
| | | | subunit 7 | | | |
| KCNMB1 | 436 | 470122 | potassium large conductance calcium- | SEQ ID No: 1094 | SEQ ID No: 1095 | SEQ ID No: 1096 |
| | | | activated channel, subfamily m, beta | | | |
| | | | member 1 | | | |
| MTCP1 | 437 | 470175 | mature t-cell proliferation 1 | SEQ ID No: 1097 | SEQ ID No: 1098 | SEQ ID No: 1099 |
| CNTNAP1 | 438 | 470279 | contactin associated protein 1 | SEQ ID No: 1100 | | SEQ ID No: 1101 |
| LOC90139 | 439 | 470819 | tetraspanin similiar to uroplakin 1 | SEQ ID No: 1102 | | SEQ ID No: 1103 |
| MRE11A | 440 | 471256 | mre11 meiotic recombination 11 | SEQ ID No: 1104 | SEQ ID No: 1105 | SEQ ID No: 1106 |
| | | | homolog a (s. cerevisiae) | | | |
| ICAM2 | 441 | 471918 | intercellular adhesion molecule 2 | SEQ ID No: 1107 | | SEQ ID No: 1108 |
| BZRP | 442 | 472021 | benzodiazapine receptor (peripheral) | SEQ ID No: 1109 | SEQ ID No: 1110 | SEQ ID No: 1111 |
| | 443 | 47986 | | | SEQ ID No: 1112 | |
| ITGB3 | 444 | 484874 | integrin, beta 3 (platelet glycoprotein | SEQ ID No: 1113 | | SEQ ID No: 1114 |
| | | | iiia, antigen cd61) | | | |
| | 445 | 485742 | similar to hypothetical protein | SEQ ID No: 1115 | SEQ ID No: 1116 | |
| | | | bc015353 | | | |
| CABC1 | 446 | 486151 | chaperone, abc1 activity of bc1 | SEQ ID No: 1117 | SEQ ID No: 1118 | SEQ ID No: 1119 |
| | | | complex like (s. pombe) | | | |
| RY1 | 447 | 486400 | putative nucleic acid binding protein ry-1 | SEQ ID No: 1120 | SEQ ID No: 1121 | SEQ ID No: 1122 |
| CDH13 | 448 | 486510 | cadherin 13, h-cadherin (heart) | SEQ ID No: 1123 | SEQ ID No: 1124 | SEQ ID No: 1125 |
| SRP19 | 449 | 486702 | signal recognition particle 19 kda | SEQ ID No: 1126 | SEQ ID No: 1127 | SEQ ID No: 1128 |
| MIF | 450 | 488144 | macrophage migration inhibitory factor | | SEQ ID No: 1129 | SEQ ID No: 1130 |
| | | | (glycosylation-inhibiting factor) | | | |
| LTBP1 | 451 | 488316 | latent transforming growth factor beta | SEQ ID No: 1131 | SEQ ID No: 1132 | SEQ ID No: 1133 |
| | | | binding protein 1 | | | |
| ZNF354A | 452 | 488412 | zinc finger protein 354a | SEQ ID No: 1134 | SEQ ID No: 1135 | SEQ ID No: 1136 |
| TLE2 | 453 | 488430 | transducin-like enhancer of split 2 | SEQ ID No: 1137 | SEQ ID No: 1138 | SEQ ID No: 1139 |
| | | | (e(sp1) homolog, drosophila) | | | |
| MYH11 | 454 | 488526 | myosin, heavy polypeptide 11, smooth | SEQ ID No: 1140 | SEQ ID No: 1141 | SEQ ID No: 1142 |
| | | | muscle | | | |
| PIP5K1A | 455 | 488875 | phosphatidylinositol-4-phosphate 5- | SEQ ID No: 1143 | SEQ ID No: 1144 | SEQ ID No: 1145 |
| | | | kinase, type i, alpha | | | |
| MFAP3 | 456 | 488913 | microfibrillar-associated protein 3 | SEQ ID No: 1146 | SEQ ID No: 1147 | SEQ ID No: 1148 |
| GTF2H4 | 457 | 489497 | general transcription factor iih, | SEQ ID No: 1149 | SEQ ID No: 1150 | SEQ ID No: 1151 |
| | | | polypeptide 4, 52 kda | | | |
| LRPPRC | 458 | 489772 | leucine-rich ppr-motif containing | SEQ ID No: 1152 | SEQ ID No: 1153 | SEQ ID No: 1154 |
| KIAA0232 | 459 | 489950 | kiaa0232 gene product | SEQ ID No: 1155 | SEQ ID No: 1156 | |
| GTF2F1 | 460 | 489961 | general transcription factor iif, | SEQ ID No: 1157 | SEQ ID No: 1158 | SEQ ID No: 1159 |
| | | | polypeptide 1, 74 kda | | | |
| PSMD3 | 461 | 490174 | proteasome (prosome, macropain) 26s | SEQ ID No: 1160 | SEQ ID No: 1161 | SEQ ID No: 1162 |
| | | | subunit, non-atpase, 3 | | | |
| DF | 462 | 491284 | d component of complement (adipsin) | SEQ ID No: 1163 | | SEQ ID No: 1164 |
| PRNP | 463 | 49691 | prion protein (p27-30) (creutzfeld-jakob | SEQ ID No: 1165 | SEQ ID No: 1166 | SEQ ID No: 1167 |
| | | | disease, gerstmann-strausler-scheinker | | | |
| | | | syndrome, fatal familial insomnia) | | | |
| | 464 | 501939 | homo sapiens transcribed sequence with | SEQ ID No: 1168 | SEQ ID No: 1169 | |
| | | | strong similarity to protein | | | |
| | | | ref: np_057457.1 (h. sapiens) ww | | | |
| | | | domain-containing oxidoreductase, | | | |
| | | | isoform 1; ww domain-containing | | | |
| | | | protein wwox; fragile site fra16d | | | |
| | | | oxidoreductase; fragile 16d oxido | | | |
| | | | reductase [homo sapiens] | | | |
| CCL11 | 465 | 502658 | chemokine (c—c motif) ligand 11 | SEQ ID No: 1170 | SEQ ID No: 1171 | SEQ ID No: 1172 |
| ARHA | 466 | 503820 | ras homolog gene family, member a | SEQ ID No: 1173 | SEQ ID No: 1174 | SEQ ID No: 1175 |
| ETFB | 467 | 504184 | electron-transfer-flavoprotein, beta | SEQ ID No: 1176 | | SEQ ID No: 1177 |
| | | | polypeptide | | | |
| ZNF3 | 468 | 504811 | zinc finger protein 3 (a8-51) | SEQ ID No: 1178 | | SEQ ID No: 1179 |
| PYGL | 469 | 505573 | phosphorylase, glycogen; liver (hers | SEQ ID No: 1180 | | SEQ ID No: 1181 |
| | | | disease, glycogen storage disease type | | | |
| | | | vi) | | | |
| PRKCB1 | 470 | 50561 | protein kinase c, beta 1 | SEQ ID No: 1182 | SEQ ID No: 1183 | SEQ ID No: 1184 |
| FNBP3 | 471 | 509515 | formin binding protein 3 | SEQ ID No: 1185 | SEQ ID No: 1186 | SEQ ID No: 1187 |
| GNG12 | 472 | 509584 | guanine nucleotide binding protein (g | SEQ ID No: 1188 | | SEQ ID No: 1189 |
| | | | protein), gamma 12 | | | |
| TAF12 | 473 | 509588 | taf12 rna polymerase ii, tata box | SEQ ID No: 1190 | SEQ ID No: 1191 | SEQ ID No: 1192 |
| | | | binding protein (tbp)-associated factor, | | | |
| | | | 20 kda | | | |
| RPL27A | 474 | 509719 | ribosomal protein l27a | SEQ ID No: 1193 | SEQ ID No: 1194 | SEQ ID No: 1195 |
| PHB | 475 | 509735 | prohibitin | SEQ ID No: 1196 | SEQ ID No: 1197 | SEQ ID No: 1198 |
| SFRS9 | 476 | 509751 | splicing factor, arginine/serine-rich 9 | SEQ ID No: 1199 | | SEQ ID No: 1200 |
| NONO | 477 | 509887 | non-pou domain containing, octamer- | SEQ ID No: 1201 | SEQ ID No: 1202 | SEQ ID No: 1203 |
| | | | binding | | | |
| CDH17 | 478 | 510130 | cadherin 17, li cadherin (liver-intestine) | SEQ ID No: 1204 | SEQ ID No: 1205 | SEQ ID No: 1206 |
| CCT5 | 479 | 510161 | chaperonin containing tcp1, subunit 5 | SEQ ID No: 1207 | | SEQ ID No: 1208 |
| | | | (epsilon) | | | |
| RRM2 | 480 | 510231 | ribonucleotide reductase m2 | SEQ ID No: 1209 | SEQ ID No: 1210 | SEQ ID No: 1211 |
| | | | polypeptide | | | |
| ENO1 | 481 | 510235 | enolase 1, (alpha) | SEQ ID No: 1212 | SEQ ID No: 1213 | SEQ ID No: 1214 |
| DKFZP564B1023 | 482 | 510354 | hypothetical protein dkfzp564b1023 | SEQ ID No: 1215 | SEQ ID No: 1216 | SEQ ID No: 1217 |
| PPEF1 | 483 | 51064 | protein phosphatase, ef hand calcium- | SEQ ID No: 1218 | SEQ ID No: 1219 | SEQ ID No: 1220 |
| | | | binding domain 1 | | | |
| CKB | 484 | 510977 | creatine kinase, brain | SEQ ID No: 1221 | SEQ ID No: 1222 | SEQ ID No: 1223 |
| TM4SF1 | 485 | 511778 | transmembrane 4 superfamily member 1 | SEQ ID No: 1224 | SEQ ID No: 1225 | SEQ ID No: 1226 |
| UBE2D3 | 486 | 512000 | ubiquitin-conjugating enzyme e2d 3 | SEQ ID No: 1227 | SEQ ID No: 1228 | SEQ ID No: 1229 |
| | | | (ubc4/5 homolog, yeast) | | | |
| MRG2 | 487 | 512333 | likely ortholog of mouse myeloid | SEQ ID No: 1230 | | |
| | | | ecotropic viral integration site-related | | | |
| | | | gene 2 | | | |
| AK5 | 488 | 512824 | adenylate kinase 5 | SEQ ID No: 1231 | | SEQ ID No: 1232 |
| | 489 | 512924 | | SEQ ID No: 1233 | SEQ ID No: 1234 | |
| | 490 | 513189 | | SEQ ID No: 1235 | | |
| GADD45A | 491 | 52065 | growth arrest and dna-damage- | SEQ ID No: 1236 | | SEQ ID No: 1237 |
| | | | inducible, alpha | | | |
| GRIA1 | 492 | 52228 | glutamate receptor, ionotropic, ampa 1 | SEQ ID No: 1238 | SEQ ID No: 1239 | SEQ ID No: 1240 |
| IDH1 | 493 | 525983 | isocitrate dehydrogenase 1 (nadp+), | SEQ ID No: 1241 | SEQ ID No: 1242 | SEQ ID No: 1243 |
| | | | soluble | | | |
| | 494 | 526038 | | SEQ ID No: 1244 | SEQ ID No: 1245 | |
| PTK2 | 495 | 52982 | ptk2 protein tyrosine kinase 2 | SEQ ID No: 1246 | SEQ ID No: 1247 | SEQ ID No: 1248 |
| CBR3 | 496 | 529844 | carbonyl reductase 3 | SEQ ID No: 1249 | SEQ ID No: 1250 | SEQ ID No: 1251 |
| COX7A2 | 497 | 529882 | cytochrome c oxidase subunit viia | SEQ ID No: 1252 | SEQ ID No: 1253 | SEQ ID No: 739 |
| | | | polypeptide 2 (liver) | | | |
| | 498 | 530034 | | SEQ ID No: 1254 | SEQ ID No: 1255 | |
| | 499 | 530037 | | SEQ ID No: 1256 | SEQ ID No: 1257 | |
| UBA52 | 500 | 530069 | ubiquitin a-52 residue ribosomal protein | SEQ ID No: 1258 | SEQ ID No: 1259 | SEQ ID No: 393 |
| | | | fusion product 1 | | | |
| COX7C | 501 | 530338 | cytochrome c oxidase subunit viic | SEQ ID No: 1260 | SEQ ID No: 1261 | SEQ ID No: 1262 |
| RPL5 | 502 | 530368 | ribosomal protein 15 | SEQ ID No: 1263 | SEQ ID No: 1264 | SEQ ID No: 1265 |
| FLIPT1 | 503 | 53061 | fly-like putative organic ion transporter 1 | SEQ ID No: 1266 | SEQ ID No: 1267 | SEQ ID No: 1268 |
| | 504 | 530744 | homo sapiens cyclophilin mrna, | SEQ ID No: 1269 | SEQ ID No: 1270 | |
| | | | complete cds | | | |
| RPL13A | 505 | 530773 | ribosomal protein l13a | SEQ ID No: 1271 | SEQ ID No: 1272 | SEQ ID No: 1273 |
| | 506 | 531366 | | SEQ ID No: 1274 | SEQ ID No: 1275 | |
| EPS15R | 507 | 531496 | epidermal growth factor receptor | SEQ ID No: 1276 | SEQ ID No: 1277 | SEQ ID No: 1278 |
| | | | substrate eps15r | | | |
| STMN1 | 508 | 53227 | stathmin 1/oncoprotein 18 | SEQ ID No: 1279 | SEQ ID No: 1280 | SEQ ID No: 1281 |
| MDH1 | 509 | 53316 | malate dehydrogenase 1, nad (soluble) | SEQ ID No: 1282 | | SEQ ID No: 1283 |
| | 510 | 53331 | loc350717 | SEQ ID No: 1284 | | |
| HCNGP | 511 | 544680 | transcriptional regulator protein | SEQ ID No: 1285 | SEQ ID No: 1286 | SEQ ID No: 1287 |
| | 512 | 544767 | | SEQ ID No: 1288 | SEQ ID No: 1289 | |
| | 513 | 544806 | | SEQ ID No: 1290 | SEQ ID No: 1291 | |
| TMSB4X | 514 | 544841 | thymosin, beta 4, x chromosome | SEQ ID No: 1292 | SEQ ID No: 1293 | SEQ ID No: 1294 |
| | 515 | 544875 | | SEQ ID No: 1295 | SEQ ID No: 1296 | |
| RPL5 | 516 | 544885 | ribosomal protein l5 | SEQ ID No: 1297 | SEQ ID No: 1298 | SEQ ID No: 1265 |
| | 517 | 545000 | | SEQ ID No: 1299 | SEQ ID No: 1300 | |
| | 518 | 545236 | | SEQ ID No: 1301 | SEQ ID No: 1302 | |
| LOC92906 | 519 | 545423 | hypothetical protein bc008217 | SEQ ID No: 1303 | SEQ ID No: 1304 | SEQ ID No: 30 |
| RPL29 | 520 | 545580 | ribosomal protein l29 | SEQ ID No: 1305 | SEQ ID No: 1306 | SEQ ID No: 1307 |
| TM9SF2 | 521 | 546351 | transmembrane 9 superfamily member 2 | SEQ ID No: 1308 | | SEQ ID No: 1309 |
| GNB2L1 | 522 | 546439 | guanine nucleotide binding protein (g | SEQ ID No: 1310 | SEQ ID No: 1311 | SEQ ID No: 1312 |
| | | | protein), beta polypeptide 2-like 1 | | | |
| WASF3 | 523 | 546460 | was protein family, member 3 | SEQ ID No: 1313 | SEQ ID No: 1314 | SEQ ID No: 1315 |
| RAB7 | 524 | 546545 | rab7, member ras oncogene family | SEQ ID No: 1316 | SEQ ID No: 1317 | SEQ ID No: 1318 |
| RPS8 | 525 | 546664 | ribosomal protein s8 | SEQ ID No: 1319 | SEQ ID No: 1320 | SEQ ID No: 1321 |
| | 526 | 546935 | | SEQ ID No: 1322 | SEQ ID No: 1323 | |
| | 527 | 547224 | | SEQ ID No: 1324 | SEQ ID No: 1325 | |
| | 528 | 547334 | | SEQ ID No: 1326 | SEQ ID No: 1327 | |
| WASL | 529 | 547443 | wiskott-aldrich syndrome-like | SEQ ID No: 1328 | | SEQ ID No: 1329 |
| RPL10A | 530 | 548702 | ribosomal protein l10a | SEQ ID No: 1330 | SEQ ID No: 1331 | SEQ ID No: 1332 |
| BOP1 | 531 | 548777 | block of proliferation 1 | SEQ ID No: 1333 | SEQ ID No: 1334 | SEQ ID No: 1335 |
| G22P1 | 532 | 549065 | thyroid autoantigen 70 kda (ku antigen) | SEQ ID No: 1336 | SEQ ID No: 1337 | SEQ ID No: 1338 |
| ARSD | 533 | 549139 | arylsulfatase d | SEQ ID No: 1339 | SEQ ID No: 1340 | SEQ ID No: 1341 |
| RPS8 | 534 | 549152 | ribosomal protein s8 | SEQ ID No: 1342 | SEQ ID No: 1343 | SEQ ID No: 1321 |
| EIF3S2 | 535 | 549173 | eukaryotic translation initiation factor 3, | SEQ ID No: 1344 | SEQ ID No: 1345 | SEQ ID No: 1346 |
| | | | subunit 2 beta, 36 kda | | | |
| YWHAQ | 536 | 549178 | tyrosine 3-monooxygenase/tryptophan | SEQ ID No: 1347 | | SEQ ID No: 1348 |
| | | | 5-monooxygenase activation protein, | | | |
| | | | theta polypeptide | | | |
| RPL5 | 537 | 549200 | ribosomal protein 15 | SEQ ID No: 1349 | SEQ ID No: 1350 | SEQ ID No: 1265 |
| NPM1 | 538 | 549212 | nucleophosmin (nucleolar | | SEQ ID No: 1351 | SEQ ID No: 1352 |
| | | | phosphoprotein b23, numatrin) | | | |
| COX5B | 539 | 549361 | cytochrome c oxidase subunit vb | SEQ ID No: 1353 | | SEQ ID No: 478 |
| PPP2CA | 540 | 550315 | protein phosphatase 2 (formerly 2a), | SEQ ID No: 1354 | SEQ ID No: 1355 | SEQ ID No: 1066 |
| | | | catalytic subunit, alpha isoform | | | |
| MYH1 | 541 | 561922 | myosin, heavy polypeptide 1, skeletal | SEQ ID No: 1356 | SEQ ID No: 1357 | SEQ ID No: 1358 |
| | | | muscle, adult | | | |
| ACTA1 | 542 | 561948 | actin, alpha 1, skeletal muscle | SEQ ID No: 1359 | SEQ ID No: 1360 | SEQ ID No: 1361 |
| TTN | 543 | 562021 | titin | SEQ ID No: 1362 | SEQ ID No: 1363 | SEQ ID No: 1364 |
| XRCC5 | 544 | 563112 | x-ray repair complementing defective | SEQ ID No: 1365 | | SEQ ID No: 1366 |
| | | | repair in chinese hamster cells 5 | | | |
| | | | (double-strand-break rejoining; ku | | | |
| | | | autoantigen, 80 kda) | | | |
| CCNB1 | 545 | 563130 | cyclin b1 | SEQ ID No: 1367 | SEQ ID No: 1368 | SEQ ID No: 1369 |
| HSPD1 | 546 | 563819 | heat shock 60 kda protein 1 (chaperonin) | SEQ ID No: 1370 | SEQ ID No: 1371 | SEQ ID No: 1372 |
| HMGB1 | 547 | 564501 | high-mobility group box 1 | SEQ ID No: 1373 | | SEQ ID No: 1374 |
| SP3 | 548 | 564535 | sp3 transcription factor | SEQ ID No: 1375 | SEQ ID No: 1376 | |
| GSTT2 | 549 | 564547 | glutathione s-transferase theta 2 | SEQ ID No: 1377 | SEQ ID No: 1378 | SEQ ID No: 1379 |
| XRCC5 | 550 | 587547 | x-ray repair complementing defective | SEQ ID No: 1380 | SEQ ID No: 1381 | SEQ ID No: 1366 |
| | | | repair in chinese hamster cells 5 | | | |
| | | | (double-strand-break rejoining; ku | | | |
| | | | autoantigen, 80 kda) | | | |
| CRNKL1 | 551 | 590592 | crn, crooked neck-like 1 (drosophila) | SEQ ID No: 1382 | SEQ ID No: 1383 | SEQ ID No: 1384 |
| UBE2C | 552 | 592041 | ubiquitin-conjugating enzyme e2c | SEQ ID No: 1385 | | SEQ ID No: 1386 |
| PPP4R2 | 553 | 592521 | protein phosphatase 4, regulatory | | SEQ ID No: 1387 | SEQ ID No: 1388 |
| | | | subunit 2 | | | |
| PDK4 | 554 | 594120 | pyruvate dehydrogenase kinase, | SEQ ID No: 1389 | | SEQ ID No: 1390 |
| | | | isoenzyme 4 | | | |
| | 555 | 594540 | similar to metallothionein-ie (mt-1e) | SEQ ID No: 1391 | | |
| BPHL | 556 | 595600 | biphenyl hydrolase-like (serine | SEQ ID No: 1392 | SEQ ID No: 1393 | SEQ ID No: 1394 |
| | | | hydrolase; breast epithelial mucin- | | | |
| | | | associated antigen) | | | |
| ZNF204 | 557 | 60204 | zinc finger protein 204 | SEQ ID No: 1395 | SEQ ID No: 1396 | |
| HOXA1 | 558 | 611075 | homeo box a1 | SEQ ID No: 1397 | SEQ ID No: 1398 | SEQ ID No: 1399 |
| C22ORF19 | 559 | 611123 | chromosome 22 open reading frame 19 | SEQ ID No: 1400 | SEQ ID No: 1401 | SEQ ID No: 1402 |
| MYF6 | 560 | 611255 | myogenic factor 6 (herculin) | SEQ ID No: 1403 | SEQ ID No: 1404 | SEQ ID No: 1405 |
| KIAA1181 | 561 | 611623 | kiaa1181 protein | SEQ ID No: 1406 | | SEQ ID No: 1407 |
| AMPD1 | 562 | 611660 | adenosine monophosphate deaminase 1 | SEQ ID No: 1408 | | SEQ ID No: 1409 |
| | | | (isoform m) | | | |
| TNNT3 | 563 | 611783 | troponin t3, skeletal, fast | SEQ ID No: 1410 | | SEQ ID No: 1411 |
| NEDD5 | 564 | 611946 | neural precursor cell expressed, | SEQ ID No: 1412 | SEQ ID No: 1413 | SEQ ID No: 1414 |
| | | | developmentally down-regulated 5 | | | |
| HSPA9B | 565 | 612365 | heat shock 70 kda protein 9b (mortalin- | SEQ ID No: 1415 | SEQ ID No: 1416 | SEQ ID No: 664 |
| | | | 2) | | | |
| | 566 | 62429 | | SEQ ID No: 1417 | SEQ ID No: 1418 | |
| | 567 | 624513 | homo sapiens transcribed sequence with | SEQ ID No: 1419 | SEQ ID No: 1420 | |
| | | | strong similarity to protein pir: s29331 | | | |
| | | | (h. sapiens) s29331 glutamate | | | |
| | | | dehydrogenase - human | | | |
| GNB2L1 | 568 | 625541 | guanine nucleotide binding protein (g | SEQ ID No: 1421 | SEQ ID No: 1422 | SEQ ID No: 1312 |
| | | | protein), beta polypeptide 2-like 1 | | | |
| GNB2L1 | 569 | 625574 | guanine nucleotide binding protein (g | SEQ ID No: 1423 | SEQ ID No: 1424 | SEQ ID No: 1312 |
| | | | protein), beta polypeptide 2-like 1 | | | |
| MYL3 | 570 | 628602 | myosin, light polypeptide 3, alkali; | SEQ ID No: 1425 | SEQ ID No: 1426 | SEQ ID No: 1427 |
| | | | ventricular, skeletal, slow | | | |
| COX6B | 571 | 632026 | cytochrome c oxidase subunit vib | SEQ ID No: 1428 | SEQ ID No: 1429 | SEQ ID No: 1430 |
| DNAJD1 | 572 | 664980 | dnaj (hsp40) homolog, subfamily d, | SEQ ID No: 1431 | | SEQ ID No: 1432 |
| | | | member 1 | | | |
| AKR1A1 | 573 | 665117 | aldo-keto reductase family 1, member | SEQ ID No: 1433 | SEQ ID No: 1434 | SEQ ID No: 1435 |
| | | | a1 (aldehyde reductase) | | | |
| MAP2K7 | 574 | 665682 | mitogen-activated protein kinase kinase 7 | SEQ ID No: 1436 | SEQ ID No: 1437 | SEQ ID No: 1438 |
| SLC7A6 | 575 | 665778 | solute carrier family 7 (cationic amino | SEQ ID No: 1439 | SEQ ID No: 1440 | SEQ ID No: 1441 |
| | | | acid transporter, y+ system), member 6 | | | |
| ANXA6 | 576 | 665818 | annexin a6 | SEQ ID No: 1442 | SEQ ID No: 1443 | SEQ ID No: 1444 |
| HIST1H4C | 577 | 667303 | histone 1, h4c | SEQ ID No: 1445 | SEQ ID No: 1446 | SEQ ID No: 1447 |
| | 578 | 66800 | | SEQ ID No: 1448 | | |
| CPSF5 | 579 | 66820 | cleavage and polyadenylation specific | SEQ ID No: 1449 | | SEQ ID No: 1450 |
| | | | factor 5, 25 kda | | | |
| | 580 | 66832 | | SEQ ID No: 1451 | | |
| | 581 | 66836 | | SEQ ID No: 1452 | | |
| GTF2E1 | 582 | 668494 | general transcription factor iie, | SEQ ID No: 1453 | SEQ ID No: 1454 | SEQ ID No: 1455 |
| | | | polypeptide 1, alpha 56 kda | | | |
| | 583 | 66895 | homo sapiens transcribed sequences | SEQ ID No: 1456 | | |
| RPS14 | 584 | 67721 | ribosomal protein s14 | SEQ ID No: 1457 | SEQ ID No: 1458 | SEQ ID No: 1459 |
| KRT23 | 585 | 67740 | keratin 23 (histone deacetylase | SEQ ID No: 1460 | SEQ ID No: 1461 | SEQ ID No: 1462 |
| | | | inducible) | | | |
| | 586 | 67776 | | SEQ ID No: 1463 | | |
| | 587 | 68140 | | SEQ ID No: 1464 | SEQ ID No: 1465 | |
| | 588 | 68141 | | SEQ ID No: 1466 | | |
| FLJ10916 | 589 | 68176 | hypothetical protein flj10916 | SEQ ID No: 1467 | SEQ ID No: 1468 | SEQ ID No: 1469 |
| ERCC4 | 590 | 682268 | excision repair cross-complementing | SEQ ID No: 1470 | SEQ ID No: 1471 | SEQ ID No: 1472 |
| | | | rodent repair deficiency, | | | |
| | | | complementation group 4 | | | |
| | 591 | 68227 | | SEQ ID No: 1473 | SEQ ID No: 1474 | |
| COL5A1 | 592 | 68276 | collagen, type v, alpha 1 | SEQ ID No: 1475 | | SEQ ID No: 1476 |
| MYOM1 | 593 | 68351 | myomesin 1 (skelemin) 185 kda | SEQ ID No: 1477 | | SEQ ID No: 1478 |
| NEK6 | 594 | 69584 | nima (never in mitosis gene a)-related | SEQ ID No: 1479 | | SEQ ID No: 1480 |
| | | | kinase 6 | | | |
| RPS23 | 595 | 70825 | ribosomal protein s23 | SEQ ID No: 1481 | SEQ ID No: 1482 | SEQ ID No: 1483 |
| RPL5 | 596 | 71096 | ribosomal protein 15 | SEQ ID No: 1484 | SEQ ID No: 1485 | SEQ ID No: 1265 |
| HSF1 | 597 | 712675 | heat shock transcription factor 1 | SEQ ID No: 1486 | SEQ ID No: 1487 | SEQ ID No: 1488 |
| FRAP1 | 598 | 713218 | fk506 binding protein 12-rapamycin | SEQ ID No: 1489 | SEQ ID No: 1490 | SEQ ID No: 1491 |
| | | | associated protein 1 | | | |
| MGC27165 | 599 | 713459 | hypothetical protein mgc27165 | SEQ ID No: 1492 | SEQ ID No: 1493 | |
| RPS27 | 600 | 72056 | ribosomal protein s27 | SEQ ID No: 1494 | SEQ ID No: 1495 | SEQ ID No: 1496 |
| | | | (metallopanstimulin 1) | | | |
| RELA | 601 | 723731 | v-rel reticuloendotheliosis viral | SEQ ID No: 1497 | | SEQ ID No: 1498 |
| | | | oncogene homolog a, nuclear factor of | | | |
| | | | kappa light polypeptide gene enhancer | | | |
| | | | in b-cells 3, p65 (avian) | | | |
| RYR3 | 602 | 72497 | ryanodine receptor 3 | SEQ ID No: 1499 | | SEQ ID No: 1500 |
| COL6A1 | 603 | 726342 | collagen, type vi, alpha 1 | SEQ ID No: 1501 | SEQ ID No: 1502 | SEQ ID No: 825 |
| CNN1 | 604 | 726779 | calponin 1, basic, smooth muscle | SEQ ID No: 1503 | | SEQ ID No: 1504 |
| ITIH1 | 605 | 72694 | inter-alpha (globulin) inhibitor, h1 | SEQ ID No: 1505 | | SEQ ID No: 1506 |
| | | | polypeptide | | | |
| PDE1A | 606 | 727792 | phosphodiesterase 1a, calmodulin- | SEQ ID No: 1507 | SEQ ID No: 1508 | SEQ ID No: 1509 |
| | | | dependent | | | |
| SSR2 | 607 | 72789 | signal sequence receptor, beta | SEQ ID No: 1510 | SEQ ID No: 1511 | SEQ ID No: 1512 |
| | | | (translocon-associated protein beta) | | | |
| NFYA | 608 | 730787 | nuclear transcription factor y, alpha | SEQ ID No: 1513 | SEQ ID No: 1514 | SEQ ID No: 1515 |
| RPS7 | 609 | 73590 | ribosomal protein s7 | SEQ ID No: 1516 | SEQ ID No: 1517 | SEQ ID No: 1518 |
| | 610 | 74834 | | SEQ ID No: 1519 | | |
| SVIL | 611 | 754018 | supervillin | SEQ ID No: 1520 | | SEQ ID No: 1521 |
| THPO | 612 | 754034 | thrombopoietin (myeloproliferative | SEQ ID No: 1522 | SEQ ID No: 1523 | SEQ ID No: 1524 |
| | | | leukemia virus oncogene ligand, | | | |
| | | | megakaryocyte growth and | | | |
| | | | development factor) | | | |
| C1ORF29 | 613 | 754479 | chromosome 1 open reading frame 29 | SEQ ID No: 1525 | SEQ ID No: 1526 | SEQ ID No: 1527 |
| IFITM1 | 614 | 755599 | interferon induced transmembrane | SEQ ID No: 1528 | SEQ ID No: 1529 | SEQ ID No: 1530 |
| | | | protein 1 (9-27) | | | |
| RARB | 615 | 755663 | retinoic acid receptor, beta | SEQ ID No: 1531 | SEQ ID No: 1532 | SEQ ID No: 398 |
| BMP6 | 616 | 768168 | bone morphogenetic protein 6 | SEQ ID No: 1533 | SEQ ID No: 1534 | SEQ ID No: 1535 |
| RPS6KB1 | 617 | 773319 | ribosomal protein s6 kinase, 70 kda, | SEQ ID No: 1536 | SEQ ID No: 1537 | SEQ ID No: 1538 |
| | | | polypeptide 1 | | | |
| R30953_1 | 618 | 782601 | hypothetical protein r30953_1 | SEQ ID No: 1539 | SEQ ID No: 1540 | SEQ ID No: 1541 |
| RNF13 | 619 | 785886 | ring finger protein 13 | SEQ ID No: 1542 | SEQ ID No: 1543 | SEQ ID No: 1544 |
| CGI-128 | 620 | 786662 | cgi-128 protein | SEQ ID No: 1545 | SEQ ID No: 1546 | SEQ ID No: 1547 |
| | 621 | 78879 | similar to complement component 3 | SEQ ID No: 1548 | | |
| CDH1 | 622 | 79598 | cadherin 1, type 1, e-cadherin | SEQ ID No: 1549 | SEQ ID No: 1550 | SEQ ID No: 1551 |
| | | | (epithelial) | | | |
| FHL3 | 623 | 796475 | four and a half lim domains 3 | SEQ ID No: 1552 | SEQ ID No: 1553 | SEQ ID No: 1554 |
| | 624 | 79829 | homo sapiens transcribed sequences | SEQ ID No: 1555 | | |
| VAV1 | 625 | 80384 | vav 1 oncogene | SEQ ID No: 1556 | SEQ ID No: 1557 | SEQ ID No: 1558 |
| PPP1R14A | 626 | 809611 | protein phosphatase 1, regulatory | SEQ ID No: 1559 | | SEQ ID No: 1560 |
| | | | (inhibitor) subunit 14a | | | |
| ETV4 | 627 | 809959 | ets variant gene 4 (e1a enhancer | SEQ ID No: 1561 | SEQ ID No: 1562 | SEQ ID No: 1563 |
| | | | binding protein, e1af) | | | |
| S100A2 | 628 | 810813 | s100 calcium binding protein a2 | SEQ ID No: 1564 | SEQ ID No: 1565 | SEQ ID No: 1566 |
| ITGA2 | 629 | 811740 | integrin, alpha 2 (cd49b, alpha 2 | SEQ ID No: 1567 | SEQ ID No: 1568 | SEQ ID No: 1569 |
| | | | subunit of vla-2 receptor) | | | |
| YWHAZ | 630 | 811939 | tyrosine 3-monooxygenase/tryptophan | SEQ ID No: 1570 | SEQ ID No: 1571 | SEQ ID No: 1572 |
| | | | 5-monooxygenase activation protein, | | | |
| | | | zeta polypeptide | | | |
| PCDH7 | 631 | 813384 | bh-protocadherin (brain-heart) | SEQ ID No: 1573 | | SEQ ID No: 1574 |
| | 632 | 813755 | similar to zinc finger protein 7 (zinc | SEQ ID No: 1575 | SEQ ID No: 1576 | |
| | | | finger protein kox4) (zinc finger protein | | | |
| | | | hf. 16) | | | |
| GJB2 | 633 | 823859 | gap junction protein, beta 2, 26 kda | SEQ ID No: 1577 | SEQ ID No: 1578 | SEQ ID No: 1579 |
| | | | (connexin 26) | | | |
| VWF | 634 | 840486 | von willebrand factor | SEQ ID No: 1580 | SEQ ID No: 1581 | SEQ ID No: 1582 |
| NME1 | 635 | 845363 | non-metastatic cells 1, protein (nm23a) | SEQ ID No: 1583 | | SEQ ID No: 288 |
| | | | expressed in | | | |
| EIF3S6 | 636 | 856961 | eukaryotic translation initiation factor 3, | SEQ ID No: 1584 | | SEQ ID No: 1585 |
| | | | subunit 6 48 kda | | | |
| | 637 | 86078 | | SEQ ID No: 1586 | | |
| | 638 | 869440 | | SEQ ID No: 1587 | | |
| RPL30 | 639 | 878681 | ribosomal protein 130 | SEQ ID No: 1588 | | SEQ ID No: 1589 |
| B2M | 640 | 878798 | beta-2-microglobulin | SEQ ID No: 1590 | | SEQ ID No: 813 |
| HMGB2 | 641 | 884365 | high-mobility group box 2 | SEQ ID No: 1591 | | SEQ ID No: 552 |
| LAMR1 | 642 | 884644 | laminin receptor 1 (ribosomal protein | SEQ ID No: 1592 | | SEQ ID No: 987 |
| | | | sa, 67 kda) | | | |
| PRAME | 643 | 897956 | preferentially expressed antigen in | SEQ ID No: 1593 | | SEQ ID No: 1594 |
| | | | melanoma | | | |
| NME2 | 644 | 951066 | non-metastatic cells 2, protein (nm23b) | SEQ ID No: 1595 | | SEQ ID No: 1596 |
| | | | expressed in | | | |

Table 1 above identifies a library of polynucleotide sequences of SEQ ID NO. 1 to SEQ ID NO. 1556 and arranges them into sets. Table 1 indicates, wherever available, the name of the gene with its gene symbol, its Image Clone and, for each gene, the relevant SEQ ID NOS defining the set. The “3′” and “5′” columns represent ESTs and the “Ref.” column represent mRNAs of the named gene or Image Clone.

Thus, the nucleotide sequences of the present invention can be defined by the differents sets, but can also be defined by the name of the gene or fragments thereof as recited in Table 1. Each polynucleotide sequence in Table 1 can therefore be considered as a marker of the corresponding gene. Each marker corresponds to a gene in the human genome; i.e., such marker is identifiable as all or a portion of a gene. The term “marker”, as used herein, is thus meant to refer to the complete gene nucleotide sequence or an EST nucleotide sequence derived from that gene (or a subsequence or complement thereof), the expression or level of which changes with certain conditions, disorders or diseases. Where the expression of the gene correlates with a certain condition, disorder or disease, the gene is a marker for that condition, disorder or disease. Any RNA transcribed from a marker gene (e.g., mRNAs), any cDNA or cRNA produced therefrom, and any nucleic acid derived therefrom, such as synthetic nucleic acid having a sequence derived from the gene corresponding to the marker gene, are also encompassed by the present invention.

Each mRNA sequence in the Ref. column represents one of the various mRNA splice forms of the gene that are known in the art; e.g., splice forms described in publicly available genomic databases. A skilled artisan is able to select, by routine experimentation, one or more appropriate splice form(s) by, e.g., determining those splice forms having a sequence that matches the sequence of the corresponding Image Clone with a predetermined level of homology.

A disease, disorder, or condition “associated with” an aberrant expression of a nucleic acid refers to a disease, disorder, or condition in a subject which is caused by, contributed to by, or causative of an aberrant level of expression of a nucleic acid.

By “nucleic acids,” as used herein, is meant polynucleotides, e.g., isolated, such as isolated deoxyribonucleic acid (DNA), and, where appropriate, isolated ribonucleic acid (RNA). The term is also understood to include, as equivalents, analogs of RNA or DNA made from nucleotide analogs, and, as applicable to the embodiment being described, single (sense or antisense) and double-stranded polynucleotides. ESTs, chromosomes or genomic DNA, cDNAs, mRNAs, and rRNAs are representative examples of molecules that can be referred to as nucleic acids. DNA can be obtained from said nucleic acids sample and RNA can be obtained by transcription of said DNA. In addition, mRNA can be isolated from said nucleic acids sample and cDNA can be obtained by reverse transcription of said mRNA.

The term “subsequence”, as used herein, is meant to refer to any sequence corresponding to a part of said polynucleotide sequence, which would also be suitable to perform the method of analysis according to the invention. A person skilled in the art can choose the position and length of a subsequence of the invention by applying routine experiments. A subsequence can have at least about 80% homology with said polynucleotide sequence; e.g., at least about 85%, at least about 90%, at least about 95%, or at least about 99% homology.

The term “pool”, as used herein, is meant to refer to a group of nucleic acid sequences comprising one or more sequences, for example about: 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500,1600, 1700, 1800, 1900, or 2000 sequences.

The number of sets may vary in the range of from 1 to the maximum number of sets described therein, e.g., 646 sets, for example about: 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 550, or 600 sets.

The over or under expression (or respectively “up regulation” and “down regulation,” which may be used interchangeably with over or under expression, respectively) can be determined by any known method within the skill in the art, such as disclosed in PCT patent application WO 02/103320, the entire disclosure of which is herein incorporated by reference. Such methods can comprise the detection of difference in the expression of the polynucleotide sequences according to the present invention in relation to at least one control. Said control can comprise, for example, polynucleotide sequence(s) from sample of the same patient or from a pool of patients exhibiting histopathologic features of colorectal disease, or selected from among reference sequence(s) which are already known to be over or under expressed. The expression level of said control can be an average or an absolute value of the expression of reference polynucleotide sequences. These values can be processed (e.g., statistically) in order to accentuate the difference relative to the expression of the polynucleotide sequences of the invention.

The analysis of the over or under expression of polynucleotide sequences can be carried out on sample, such as biological material derived from any mammalian cells, including cell lines, xenografts, and human tissues, preferably from colon tissue. The method according to the invention can be performed on sample from a human subject or an animal (for example for veterinary application or preclinical trial).

By “over or underexpression” of a polynucleotide sequence, as used herein, is meant that overexpression of certain sequences is detected simultaneously with the underexpression of other sequences. “Simultaneously” means concurrent with or within a biologic or functionally relevant period of time during which the over expression of a sequence can be followed by the under expression of another sequence, or conversely, e.g., because both over and under expression are directly or indirectly correlated.

In one embodiment, the method according to the present invention is therefore directed to the analysis of differential gene expression associated with colon tumors wherein the pool of polynucleotide sequences corresponds to all or part of the polynucleotide sequences, subsequences or complements thereof, selected from each of predefined polynucleotide sequence sets consisting of sets:

1; 4; 9; 10; 11; 13; 15; 16; 17; 18; 21; 27; 28; 30; 31; 34; 37; 39; 41; 43; 45; 46; 52; 53; 58; 59; 60; 65; 68; 69; 70; 75; 76; 78; 79; 80; 84; 85; 87; 88; 90; 95; 96; 98; 99; 101; 105; 108; 110; 111; 113; 114; 116; 119; 120; 122; 124; 125; 126; 127; 130; 131; 138; 139; 140; 141; 143; 150; 152; 153; 155; 159; 164; 171; 175; 176; 178; 181; 182; 184; 185; 189; 192; 196; 197; 198; 203; 205; 207; 208; 210; 213; 214; 215; 216; 218; 221; 223; 225; 227; 231; 235; 241; 243; 251; 256; 259; 261; 262; 263; 264; 266; 267; 268; 270; 279; 281; 286; 287; 288; 291; 298; 299; 301; 307; 310; 312; 313; 317; 319; 329; 331; 332; 337; 338; 339; 340; 341; 342; 344; 346; 352; 354; 357; 360; 361; 366; 368; 369; 377; 379; 381; 384; 385; 386; 390; 392; 394; 395; 397; 398; 400; 401; 405; 406; 409; 410; 413; 423; 427; 434; 436; 437; 438; 440; 442; 443; 444; 445; 448; 454; 459; 463; 464; 467; 469; 470; 488; 492; 495; 500; 503; 507; 508; 516; 518; 520; 522; 524; 538; 543; 547; 549; 552; 555; 557; 561; 567; 568; 569; 573; 574; 583; 586; 588; 592; 596; 597; 598; 599; 600; 601; 604; 609; 610; 611; 614; 616; 617; 621; 626; 627; 629; 630; 631; 632; 634; 635; 636; 638; 641; 642; and 644.

Said analysis can comprise at least one of the following steps:
The detection of the overexpression of a pool of polynucleotide sequences in colon tissues, said pool corresponding to all or part of the polynucleotide sequences, subsequences or complements thereof, selected from each of predefined polynucleotide sequences sets consisting of sets:

1; 9; 10; 16; 18; 27; 28; 30; 39; 41; 43; 45; 53; 58; 60; 65; 69; 75; 76; 113; 116; 120; 122; 126; 127; 130; 131; 138; 139; 140; 141; 143; 150; 152; 153; 159; 181; 182; 184; 189; 192; 197; 198; 210; 213; 214; 216; 218; 225; 227; 243; 259; 261; 264; 266; 267; 268; 281; 286; 287; 288; 291; 299; 307; 312; 313; 317; 319; 332; 337; 338; 339; 340; 341; 342; 344; 354; 357; 360; 361; 368; 381; 384; 385; 392; 394; 397; 398; 405; 423; 427; 442; 444; 464; 467; 469; 488; 495; 500; 507; 508; 516; 520; 522; 524; 538; 543; 547; 549; 552; 561; 567; 568; 569; 573; 586; 588; 592; 596; 600; 609; 614; 627; 629; 630; 635; 636; 641; 642; and 644.
The detection of the underexpression of a pool of polynucleotide sequences in colon tissues, said pool corresponding to all or part of the polynucleotide sequences, subsequences or complements thereof, selected from each of predefined polynucleotide sequence sets consisting of sets:

4; 11; 13; 15; 17; 21; 31; 34; 37; 46; 52; 59; 68; 70; 78; 79; 80; 84; 85; 87; 88; 90; 95; 96; 98; 99; 101; 105; 108; 110; 111; 114; 119; 124; 125; 155; 164; 171; 175; 176; 178; 185; 196; 203; 205; 207; 208; 215; 221; 223; 231; 235; 241; 251; 256; 262; 263; 270; 279; 298; 301; 310; 329; 331; 346; 352; 366; 369; 377; 379; 386; 390; 395; 400; 401; 406; 409; 410; 413; 434; 436; 437; 438; 440; 443; 445; 448; 454; 459; 463; 470; 492; 503; 518; 555; 557; 574; 583; 597; 598; 599; 601; 604; 610; 611; 616; 617; 621; 626; 631; 632; 634; and 638.

In a preferred embodiment, the sets for analyzing differential gene expression associated with colon tumors can, for example, consist of those mentioned in Table 2:

TABLE 2

| | Clone | | | | | |
|---|---|---|---|---|---|---|
| | identifier | | Gene | Reference | Title of cluster | |
| Sets | (Image) | Cluster (Unigene) | Symbol | sequences | (Gene name) | SEQ ID Numbers |
| 1 | 1012666 | ughs.82422:175 | capg | nm_001747 | capping protein (actin filament), | SEQ ID NO: 1597 |
| | | | | | gelsolin-like | |
| 4 | 1046837 | ughs.235935:175 | nov | nm_002514 | nephroblastoma overexpressed gene | SEQ ID NO: 1598 |
| 15 | 110486 | ughs.404336:175 | loc92906 | nm_138394 | hypothetical protein bc008217 | SEQ ID NO: 1599 |
| 21 | 117240 | ughs.180398:175 | lpp | nm_005578 | lim domain containing preferred | SEQ ID NO: 1600 |
| | | | | | translocation partner in lipoma | |
| 27 | 119530 | ughs.17287:175 | kcnj15 | nm_002243, | potassium inwardly-rectifying | SEQ ID NO: 1601 |
| | | | | nm_170736, | channel, subfamily j, member 15 | SEQ ID NO: 1602 |
| | | | | nm_170737 | | SEQ ID NO: 1603 |
| 58 | 1338831 | | | | | |
| 68 | 139789 | ughs.79095:175 | eps15 | nm_001981 | epidermal growth factor receptor | SEQ ID NO: 1604 |
| | | | | | pathway substrate 15 | |
| 75 | 1456160 | ughs.531989:175 | azgp1 | nm_001185 | alpha-2-glycoprotein 1, zinc | SEQ ID NO: 1605 |
| 79 | 146922 | | | | | |
| 95 | 153461 | ughs.25511:175 | tgfb1i1 | nm_015927 | transforming growth factor beta 1 | SEQ ID NO: 1606 |
| | | | | | induced transcript 1 | |
| 98 | 153854 | ughs.279604:175 | des | nm_001927 | desmin | SEQ ID NO: 1607 |
| 101 | 154600 | ughs.80776:175 | plcd1 | nm_006225 | phospholipase c, delta 1 | SEQ ID NO: 1608 |
| 114 | 1667886 | ughs.75486:175 | hsf4 | nm_001538 | heat shock transcription factor 4 | SEQ ID NO: 1609 |
| 119 | 1731982 | ughs.271620:175 | plcg2 | nm_002661 | phospholipase c, gamma 2 | SEQ ID NO: 1610 |
| | | | | | (phosphatidylinositol-specific) | |
| 127 | 186331 | ughs.32393:175 | dars | nm_001349 | aspartyl-trna synthetase | SEQ ID NO: 1611 |
| 131 | 1912132 | ughs.250822:175 | stk6 | nm_003600, | serine/threonine kinase 6 | SEQ ID NO: 1612 |
| | | | | nm_198433, | | SEQ ID NO: 1613 |
| | | | | nm_198434, | | SEQ ID NO: 1614 |
| | | | | nm_198435, | | SEQ ID NO: 1615 |
| | | | | nm_198436, | | SEQ ID NO: 1616 |
| | | | | nm_198437 | | SEQ ID NO: 1617 |
| 140 | 195702 | ughs.270920:175 | dap3 | nm_004632, | death associated protein 3 | SEQ ID NO: 1618 |
| | | | | nm_033657 | | SEQ ID NO: 1619 |
| 155 | 2055272 | ughs.252938:175 | lrp2 | nm_004525 | low density lipoprotein-related | SEQ ID NO: 1620 |
| | | | | | protein 2 | |
| 176 | 2349125 | ughs.136713:175 | vpreb3 | nm_013378 | pre-b lymphocyte gene 3 | SEQ ID NO: 1621 |
| 192 | 241788 | ughs.300774:175 | fgb | nm_005141 | fibrinogen, b beta polypeptide | SEQ ID NO: 1622 |
| 241 | 272189 | ughs.260523:175 | nras | nm_002524 | neuroblastoma ras viral (v-ras) | SEQ ID NO: 1623 |
| | | | | | oncogene homolog | |
| 243 | 272502 | ughs.374334:175 | cct4 | nm_006430 | chaperonin containing tcp1, subunit 4 | SEQ ID NO: 1624 |
| | | | | | (delta) | |
| 259 | 285780 | ughs.2936:175 | mmp13 | nm_002427 | matrix metalloproteinase 13 | SEQ ID NO: 1625 |
| | | | | | (collagenase 3) | |
| 263 | 288874 | ughs.37014:175; | ca7; | nm_005182; | carbonic anhydrase vii; zinc finger | SEQ ID NO: 1626 |
| | | ughs.48589:175 | znf228 | nm_013380 | protein 228 | SEQ ID NO: 1627 |
| 270 | 30066 | ughs.89657:175 | ilk | nm_004517 | integrin-linked kinase | SEQ ID NO: 1628 |
| 279 | 306697 | ughs.82508:175 | thap11 | nm_020457 | thap domain containing 11 | SEQ ID NO: 1629 |
| 286 | 310860 | ughs.368481:175 | nudt5 | nm_014142 | nudix (nucleoside diphosphate linked | SEQ ID NO: 1630 |
| | | | | | moiety x)-type motif 5 | |
| 298 | 322452 | ughs.124411:175 | chga | nm_001275 | chromogranin a (parathyroid | SEQ ID NO: 1631 |
| | | | | | secretory protein 1) | |
| 299 | 322471 | ughs.1063:175 | snrpc | nm_003093 | small nuclear ribonucleoprotein | SEQ ID NO: 1632 |
| | | | | | polypeptide c | |
| 307 | 323948 | ughs.2316:175 | sox9 | nm_000346 | sry (sex determining region y)-box 9 | SEQ ID NO: 1633 |
| | | | | | (campomelic dysplasia, autosomal | |
| | | | | | sex-reversal) | |
| 310 | 324369 | ughs.513557:175 | ctbs | nm_004388 | chitobiase, di-n-acetyl- | SEQ ID NO: 1634 |
| 312 | 324757 | ughs.370504:175 | rps15a | nm_001019 | ribosomal protein s15a | SEQ ID NO: 1635 |
| 313 | 324930 | ughs.28491:175 | sat | nm_002970 | spermidine/spermine n1- | SEQ ID NO: 1636 |
| | | | | | acetyltransferase | |
| 317 | 327684 | ughs.148090:175 | cdh15 | nm_004933 | cadherin 15, m-cadherin (myotubule) | SEQ ID NO: 1637 |
| 329 | 342054 | ughs.20136:175 | cxorf6 | nm_005491 | chromosome x open reading frame 6 | SEQ ID NO: 1638 |
| 346 | 34888 | ughs.489521:175; | reln; | nm_005045, | reelin; transcribed locus | SEQ ID NO: 1639 |
| | | ughs.492257:175 | | nm_173054; | | SEQ ID NO: 1640 |
| 357 | 358117 | ughs.2316:175 | sox9 | nm_000346 | sry (sex determining region y)-box 9 | |
| | | | | | (campomelic dysplasia, autosomal | |
| | | | | | sex-reversal) | |
| 360 | 358683 | ughs.133892:175 | tpm1 | nm_000366 | tropomyosin 1 (alpha) | SEQ ID NO: 1641 |
| 361 | 358943 | ughs.438837:175 | n2n | nm_203458 | similar to notch2 protein | SEQ ID NO: 1642 |
| 394 | 383433 | ughs.356261:175 | | | similar to laminin receptor 1 | |
| 395 | 39593 | ughs.12409:175 | sst | nm_001048 | somatostatin | SEQ ID NO: 1643 |
| 398 | 39972 | ughs.432317:175 | adam23 | nm_003812 | a disintegrin and metalloproteinase | SEQ ID NO: 1644 |
| | | | | | domain 23 | |
| 405 | 415389 | ughs.334612:175 | snrpe | nm_003094 | small nuclear ribonucleoprotein | SEQ ID NO: 1645 |
| | | | | | polypeptide e | |
| 406 | 416060 | ughs.440934:175 | arg1 | nm_000045 | arginase, liver | SEQ ID NO: 1646 |
| 413 | 427858 | ughs.508411:175 | gpc6 | nm_005708 | glypican 6 | SEQ ID NO: 1647 |
| 427 | 44152 | ughs.1708:175 | cct3 | nm_005998 | chaperonin containing tcp1, subunit 3 | SEQ ID NO: 1648 |
| | | | | | (gamma) | |
| 436 | 470122 | ughs.93841:175 | kcnmb1 | nm_004137 | potassium large conductance | SEQ ID NO: 1649 |
| | | | | | calcium-activated channel, subfamily | |
| | | | | | m, beta member 1 | |
| 437 | 470175 | ughs.3548:175 | mtcp1 | nm_014221 | mature t-cell proliferation 1 | SEQ ID NO: 1650 |
| 438 | 470279 | ughs.408730:175 | cntnap1 | nm_003632 | contactin associated protein 1 | SEQ ID NO: 1651 |
| 443 | 47986 | ughs.149609:175 | itga5 | nm_002205 | integrin, alpha 5 (fibronectin | SEQ ID NO: 1652 |
| | | | | | receptor, alpha polypeptide) | |
| 454 | 488526 | ughs.78344:175 | myh11 | nm_002474, | myosin, heavy polypeptide 11, | SEQ ID NO: 1653 |
| | | | | nm_022844 | smooth muscle | SEQ ID NO: 1654 |
| 464 | 501939 | ughs.21635:175; | tubg1; | nm_001070; | tubulin, gamma 1; ww domain | SEQ ID NO: 1655 |
| | | ughs.461453:175 | wwox | nm_016373, | containing oxidoreductase | SEQ ID NO: 1656 |
| | | | | nm_018560, | | SEQ ID NO: 1657 |
| | | | | nm_130788, | | SEQ ID NO: 1658 |
| | | | | nm_130790, | | SEQ ID NO: 1659 |
| | | | | nm_130791, | | SEQ ID NO: 1660 |
| | | | | nm_130792, | | SEQ ID NO: 1661 |
| | | | | nm_130844 | | SEQ ID NO: 1662 |
| 507 | 531496 | ughs.292072:175 | eps15l1 | nm_021235 | epidermal growth factor receptor | SEQ ID NO: 1663 |
| | | | | | pathway substrate 15-like 1 | |
| 522 | 546439 | ughs.5662:175 | gnb2l1 | nm_006098 | guanine nucleotide binding protein (g | SEQ ID NO: 1664 |
| | | | | | protein), beta polypeptide 2-like 1 | |
| 547 | 564501 | ughs.434102:175 | hmgb1 | nm_002128 | high-mobility group box 1 | SEQ ID NO: 1665 |
| 552 | 592041 | ughs.93002:175 | ube2c | nm_007019, | ubiquitin-conjugating enzyme e2c | SEQ ID NO: 1666 |
| | | | | nm_181799, | | SEQ ID NO: 1667 |
| | | | | nm_181800, | | SEQ ID NO: 1668 |
| | | | | nm_181801, | | SEQ ID NO: 1669 |
| | | | | nm_181802, | | SEQ ID NO: 1670 |
| | | | | nm_181803 | | SEQ ID NO: 1671 |
| 555 | 594540 | ughs.454253:175 | ptch | nm_000264 | patched homolog (drosophila) | SEQ ID NO: 1672 |
| 568 | 625541 | ughs.5662:175 | gnb2l1 | nm_006098 | guanine nucleotide binding protein (g | |
| | | | | | protein), beta polypeptide 2-like 1 | |
| 569 | 625574 | ughs.5662:175 | gnb2l1 | nm_006098 | guanine nucleotide binding protein (g | |
| | | | | | protein), beta polypeptide 2-like 1 | |
| 614 | 755599 | ughs.458414:175 | ifitm1 | nm_003641 | interferon induced transmembrane | SEQ ID NO: 1673 |
| | | | | | protein 1 (9-27) | |
| 631 | 813384 | ughs.443020:175 | pcdh7 | nm_002589, | bh-protocadherin (brain-heart) | SEQ ID NO: 1674 |
| | | | | nm_032456, | | SEQ ID NO: 1675 |
| | | | | nm_032457 | | SEQ ID NO: 1676 |
| 634 | 840486 | ughs.440848:175 | vwf | nm_000552 | von willebrand factor | SEQ ID NO: 1677 |
| 636 | 856961 | ughs.405590:175 | eif3s6 | nm_001568 | eukaryotic translation initiation | SEQ ID NO: 1678 |
| | | | | | factor 3, subunit 6 48 kda | |
| 641 | 884365 | ughs.434953:175 | hmgb2 | nm_002129 | high-mobility group box 2 | SEQ ID NO: 1679 |
| 644 | 951066 | ughs.433416:175 | nme2 | nm_002512 | non-metastatic cells 2, protein | SEQ ID NO: 1680 |
| | | | | | (nm23b) expressed in | |

In another embodiment, the method according to the present invention is directed to the analysis of differential gene expression associated with secondary metastatic events in patients with colorectal tumors, in particular visceral metastasis or lymph node metastasis. In the visceral metastasis embodiment, said analysis comprises the detection of the overexpression or the underexpression of a pool of polynucleotide sequences in colon tissues, said pool corresponding to all or part of the polynucleotide sequences, subsequences or complements thereof, selected from each of predefined polynucleotide sequence sets consisting of sets:

2; 3; 10; 22; 24; 25; 30; 32; 33; 35; 36; 39; 40; 41; 42; 47; 50; 54; 57; 67; 72; 86; 97; 102; 103; 104; 107; 117; 118; 120; 128; 130; 132; 133; 134; 137; 144; 145; 146; 147; 149; 153; 156; 158; 162; 163; 165; 169; 170; 173; 174; 179; 180; 188; 191; 193; 194; 195; 199; 200; 201; 202; 204; 206; 209; 210; 211; 212; 213; 214; 216; 217; 219; 222; 234; 238; 246; 248; 249; 250; 255; 271; 272; 273; 276; 277; 278; 282; 283; 284; 291; 292; 293; 294; 295; 296; 303; 304; 305; 306; 308; 312; 314; 318; 323; 324; 325; 326; 330; 336; 337; 338; 339; 340; 341; 342; 343; 344; 347; 349; 350; 351; 353; 356; 359; 360; 361; 362; 363; 364; 371; 372; 374; 378; 380; 381; 382; 383; 384; 387; 388; 393; 396; 397; 399; 402; 403; 408; 414; 415; 417; 418; 419; 420; 421; 422; 426; 428; 430; 432; 433; 441; 446; 449; 457; 458; 460; 465; 471; 472; 473; 475; 476; 478; 480; 481; 482; 484; 485; 486; 490; 493; 494; 497; 501; 502; 504; 505; 509; 510; 514; 516; 520; 525; 526; 527; 528; 529; 530; 537; 538; 539; 541; 545; 546; 550; 558; 559; 560; 561; 562; 564; 565; 566; 571; 576; 577; 578; 580; 581; 584; 585; 586; 590; 591; 593; 594; 595; 596; 602; 607; 609; 612; 613; 615; 623; 624; 625; 633; 635; 639; 640; 643; and 644.

The analysis can comprise at least one of the following steps:
The detection of the overexpression of a pool of polynucleotide sequences in colon tissues, said pool corresponding to all or part of the polynucleotide sequences, subsequences or complement thereof selected from each of predefined polynucleotide sequence sets consisting of sets:

36; 86; 104; 107; 117; 132; 144; 153; 156; 174; 191; 209; 248; 349; 350; 396; 417; 419; 432; 558; 566; 613; 623; 625; 633; and 643.
The detection of the underexpression of a pool of polynucleotide sequences in colon tissues, said pool corresponding to all or part of the polynucleotide sequences, subsequences or complements thereof, selected in each of predefined polynucleotide sequence sets consisting of sets:

2; 3; 10; 22; 24; 25; 30; 32; 33; 35; 39; 40; 41; 42; 47; 50; 54; 57; 67; 72; 97; 102; 103; 118; 120; 128; 130; 133; 134; 137; 145; 146; 147; 149; 158; 162; 163; 165; 169; 170; 173; 179; 180; 188; 193; 194; 195; 199; 200; 201; 202; 204; 206; 210; 211; 212; 213; 214; 216; 217; 219; 222; 234; 238; 246; 249; 250; 255; 271; 272; 273; 276; 277; 278; 282; 283; 284; 291; 292; 293; 294; 295; 296; 303; 304; 305; 306; 308; 312; 314; 318; 323; 324; 325; 326; 330; 336; 337; 338; 339; 340; 341; 342; 343; 344; 347; 351; 353; 356; 359; 360; 361; 362; 363; 364; 371; 372; 374; 378; 380; 381; 382; 383; 384; 387; 388; 393; 397; 399; 402; 403; 408; 414; 415; 418; 420; 421; 422; 426; 428; 430; 433; 441; 446; 449; 457; 458; 460; 465; 471; 472; 473; 475; 476; 478; 480; 481; 482; 484; 485; 486; 490; 493; 494; 497; 501; 502; 504; 505; 509; 510; 514; 516; 520; 525; 526; 527; 528; 529; 530; 537; 538; 539; 541; 545; 546; 550; 559; 560; 561; 562; 564; 565; 571; 576; 577; 578; 580; 581; 584; 585; 586; 590; 591; 593; 594; 595; 596; 602; 607; 609; 612; 615; 624; 635; 639; 640; and 644.

In a preferred embodiment, the sets for analyzing differential gene expression associated with visceral metastasis can, for example, consist of those mentioned in Table 3:

TABLE 3

| | Clone | | Gene | Reference | | |
|---|---|---|---|---|---|---|
| Set | identifier | cluster | Symbol | sequences | Title of cluster | SEQ ID Numbers |
| 32 | image: 121076 | ughs.107476:175; | atp5l; | nm_006476; | atp synthase, h+ transporting, | SEQ ID NO: 1681 |
| | | ughs.75275:175 | ube4a | nm_004788 | mitochondrial f0 complex, subunit g; | SEQ ID NO: 1682 |
| | | | | | ubiquitination factor e4a (ufd2 | |
| | | | | | homolog, yeast) | |
| 33 | image: 121265 | ughs.181315:175 | Ifnar1 | nm_000629 | interferon (alpha, beta and omega) | SEQ ID NO: 1683 |
| | | | | | receptor 1 | |
| 50 | image: 129146 | ughs.423404:175 | cox7a2l | nm_004718 | cytochrome c oxidase subunit viia | SEQ ID NO: 1684 |
| | | | | | polypeptide 2 like | |
| 133 | image: 191714 | ughs.370504:175; | rps15a; | nm_001019; | ribosomal protein s15a; transcribed | |
| | | ughs.486908:175 | | | locus, moderately similar to | |
| | | | | | xp_212877.2 ribosomal protein s15a | |
| | | | | | [rattus norvegicus] | |
| 188 | image: 240753 | | | | | |
| 217 | image: 258313 | ughs.432170:175 | cox7b | nm_001866 | cytochrome c oxidase subunit viib | SEQ ID NO: 1685 |
| 271 | image: 301119 | ughs.80691:175 | ckmt2 | nm_001825 | creatine kinase, mitochondrial 2 | SEQ ID NO: 1686 |
| | | | | | (sarcomeric) | |
| 284 | image: 31027 | ughs.180414:175; | hspa8; | nm_006597, | heat shock 70 kda protein 8; fragile x | SEQ ID NO: 1687 |
| | | ughs.52788:175 | fxr2 | nm_153201; | mental retardation, autosomal | SEQ ID NO: 1688 |
| | | | | nm_004860 | homolog 2 | SEQ ID NO: 1689 |
| 296 | image: 321973 | ughs.108957:175 | rps27l | nm_015920 | ribosomal protein s27-like | SEQ ID NO: 1690 |
| 303 | image: 323681 | ughs.11156:175 | loc51255 | nm_016494 | hypothetical protein loc51255 | SEQ ID NO: 1691 |
| 312 | image: 324757 | ughs.370504:175 | rps15a | nm_001019 | ribosomal protein s15a | |
| 323 | image: 33794 | ughs.155433:175 | atp5c1 | nm_001001973, | atp synthase, h+ transporting, | SEQ ID NO: 1692 |
| | | | | nm_005174 | mitochondrial f1 complex, gamma | SEQ ID NO: 1693 |
| | | | | | polypeptide 1 | |
| 340 | image: 345694 | ughs.156316:175 | Dcn | nm_001920, | decorin | SEQ ID NO: 1694 |
| | | | | nm_133503, | | SEQ ID NO: 1695 |
| | | | | nm_133504, | | SEQ ID NO: 1696 |
| | | | | nm_133505, | | SEQ ID NO: 1697 |
| | | | | nm_133506, | | SEQ ID NO: 1698 |
| | | | | nm_133507 | | SEQ ID NO: 1699 |
| 343 | image: 346269 | ughs.420269:175 | col6a2 | nm_001849, | collagen, type vi, alpha 2 | SEQ ID NO: 1700 |
| | | | | nm_058174, | | SEQ ID NO: 1701 |
| | | | | nm_058175 | | SEQ ID NO: 1702 |
| 361 | image: 358943 | ughs.438837:175 | n2n | nm_203458 | similar to notch2 protein | SEQ ID NO: 1703 |
| 403 | image: 41411 | ughs.184582:175; | rpl24; | nm_000986; | ribosomal protein l24; transcribed | SEQ ID NO: 1704 |
| | | ughs.206520:175 | | | locus | |
| 408 | image: 416946 | ughs.395309:175 | Txn | nm_003329 | thioredoxin | SEQ ID NO: 1705 |
| 473 | image: 509588 | ughs.421646:175 | taf12 | nm_005644 | taf12 rna polymerase ii, tata box | SEQ ID NO: 1706 |
| | | | | | binding protein (tbp)-associated | |
| | | | | | factor, 20 kda | |
| 484 | image: 510977 | ughs.173724:175 | Ckb | nm_001823 | creatine kinase, brain | SEQ ID NO: 1707 |
| 494 | image: 526038 | ughs.536668:175 | | | transcribed locus | |
| 502 | image: 530368 | ughs.469653:175 | rpl5 | nm_000969 | ribosomal protein l5 | SEQ ID NO: 1708 |
| 516 | image: 544885 | ughs.469653:175 | rpl5 | nm_000969 | ribosomal protein l5 | SEQ ID NO: 1708 |
| 624 | image: 79829 | ughs.7888:175 | erbb4 | nm_005235 | v-erb-a erythroblastic leukemia viral | SEQ ID NO: 1709 |
| | | | | | oncogene homolog 4 (avian) | |

According to the lymph node metastasis embodiment, said analysis comprises the detection of the overexpression or the underexpression of a pool of polynucleotide sequences in colon tissues, said pool corresponding to all or part of the polynucleotide sequences, subsequences or complements thereof, selected from each of predefined polynucleotide sequence sets consisting of sets:

38; 55; 66; 91; 93; 102; 103; 133; 142; 144; 153; 163; 190; 210; 232; 254; 280; 296; 300; 304; 311; 321; 335; 378; 383; 384; 420; 425; 429; 432; 468; 473; 487; 516; 519; 544; 553; 573; 577; 578; 585; 587; 589; 592; 605; 608; and 644; preferably from sets 142; 144; 153; 190; 280; 468; 519; 553; and 589.

The analysis can comprise at least one of the following steps:
The detection of the overexpression of a pool of polynucleotide sequences in colon tissues, said pool corresponding to all or part of the polynucleotide sequences, subsequences or complements thereof selected from each of predefined polynucleotide sequence sets consisting of sets:

55; 66; 144; 153; 432; 553; and 608; preferably 144; 153; and 553.
The detection of the underexpression of a pool of polynucleotide sequences in colon tissues, said pool corresponding to all or part of the polynucleotide sequences, subsequences or complements thereof, selected from each of predefined polynucleotide sequence sets consisting of sets:

38; 91; 93; 102; 103; 133; 142; 163; 190; 210; 232; 254; 280; 296; 300; 304; 311; 321; 335; 378; 383; 384; 420; 425; 429; 468; 473; 487; 516; 519; 544; 573; 577; 578; 585; 587; 589; 592; 605; and 644, preferably 142; 190; 280; 468; 519; and 589.

In a further preferred embodiment, the sets for analyzing differential gene expression associated with lymph node metastasis can, for example, consist of those mentioned in Table 4:

TABLE 4

| | Clone | | Gene | Reference | | |
|---|---|---|---|---|---|---|
| Set | identifier | Cluster | Symbol | sequences | Title of cluster | SEQ ID Numbers |
| 142 | Image: 198903 | ughs.418533:175 | bub3 | nm_004725 | bub3 budding uninhibited by | SEQ ID NO: 1710 |
| | | | | | benzimidazoles 3 homolog (yeast) | |
| 144 | Image: 200521 | ughs.442936:175 | oas1 | nm_002534, | 2′,5′-oligoadenylate synthetase 1, | SEQ ID NO: 1711 |
| | | | | nm_016816 | 40/46 kda | SEQ ID NO: 1712 |
| 153 | Image: 2048801 | ughs.439109:175 | ntrk2 | nm_006180 | neurotrophic tyrosine kinase, | SEQ ID NO: 1713 |
| | | | | | receptor, type 2 | |
| 190 | Image: 241151 | ughs.432424:175 | tpp2 | nm_003291 | tripeptidyl peptidase ii | SEQ ID NO: 1714 |
| 280 | Image: 307094 | ughs.54609:175 | gcat | nm_014291 | glycine c-acetyltransferase (2-amino- | SEQ ID NO: 1715 |
| | | | | | 3-ketobutyrate coenzyme a ligase) | |
| 468 | Image: 504811 | ughs.20082:175 | znf38 | nm_017715, | zinc finger protein 38 | SEQ ID NO: 1716 |
| | | | | nm_145914 | | SEQ ID NO: 1717 |
| 553 | Image: 592521 | ughs.446590:175; | ppp4r2; | nm_174907; | protein phosphatase 4, regulatory | SEQ ID NO: 1718 |
| | | ughs.534524:175 | flj10213 | nm_018029 | subunit 2; hypothetical protein | SEQ ID NO: 1719 |
| | | | | | flj10213 | |
| 589 | Image: 68176 | ughs.179203:175 | flj10916 | nm_018271 | hypothetical protein flj10916 | SEQ ID NO: 1720 |

In a further embodiment, the method of the present invention is directed to the analysis of differential gene expression associated with MSI phenotype in colon cancer. In this embodiment, said analysis comprises the detection of the overexpression or the underexpression of a pool of polynucleotide sequences in colon tissues, said pool corresponding to all or part of the polynucleotide sequences subsequences or complements thereof, selected from each of predefined polynucleotide sequence sets consisting of sets:

29; 48; 56; 62; 71; 77; 82; 109; 112; 135; 136; 154; 157; 166; 167; 186; 220; 226; 236; 237; 239; 240; 242; 244; 253; 260; 277; 290; 297; 348; 358; 375; 376; 404; 407; 412; 416; 424; 431; 450; 451; 452; 462; 474; 477; 479; 486; 498; 511; 521; 533; 534; 535; 542; 572; 619; and 622.

The analysis can comprise at least one of the following steps:
The detection of the overexpression of a pool of polynucleotide sequences in colon tissues, said pool corresponding to all or part of the polynucleotide sequences, subsequences or complements thereof selected from each of predefined polynucleotide sequence sets consisting of sets:

48; 56; 62; 157; 186; 220; 226; 253; 260; 376; 450; 452; 462; 498; and 511.
The detection of the underexpression of a pool of polynucleotide sequences in colon tissues, said pool corresponding to all or part of the polynucleotide sequences, subsequences or complements thereof, selected from each of predefined polynucleotide sequence sets consisting of sets:

29; 71; 77; 82; 109; 112; 135; 136; 154; 166; 167; 236; 237; 239; 240; 242; 244; 277; 290; 297; 348; 358; 375; 404; 407; 412; 416; 424; 431; 451; 474; 477; 479; 486; 521; 533; 534; 535; 542; 572; 619; and 622.

In a preferred embodiment, the sets for analyzing differential gene expression associated with MSI phenotype can, for example, consist of those mentioned in Table 5:

TABLE 5

| | Clone | | Gene | Reference | | |
|---|---|---|---|---|---|---|
| Set | identifier | Cluster | Symbol | sequences | Title of cluster | SEQ ID Numbers |
| 29 | Image: 120009 | Ughs.77578:175 | usp9x | nm_004652, | ubiquitin specific protease 9, x- | SEQ ID NO: 1721 |
| | | | | nm_021906 | linked (fat facets-like, drosophila) | SEQ ID NO: 1722 |
| 62 | image: 136361 | Ughs.519034:175; | tnfsf13 | nm_003808, | transcribed locus; tumor necrosis | SEQ ID NO: 1723 |
| | | ughs.54673:175 | | nm_003809, | factor (ligand) superfamily, member | SEQ ID NO: 1724 |
| | | | | nm_153012, | 12 | SEQ ID NO: 1725 |
| | | | | nm_172087, | | SEQ ID NO: 1726 |
| | | | | nm_172088, | | SEQ ID NO: 1727 |
| | | | | nm_172089 | | SEQ ID NO: 1728 |
| 71 | image: 143519 | Ughs.227729:175 | fkbp2 | nm_004470, | fk506 binding protein 2, 13 kda | SEQ ID NO: 1729 |
| | | | | nm_057092 | | SEQ ID NO: 1730 |
| 109 | image: 159885 | Ughs.298469:175 | ace | nm_000789, | angiotensin i converting enzyme | SEQ ID NO: 1731 |
| | | | | nm_152830, | (peptidyl-dipeptidase a) 1 | SEQ ID NO: 1732 |
| | | | | nm_152831 | | SEQ ID NO: 1733 |
| 136 | image: 192581 | Ughs.437040:175 | ptpn21 | nm_007039 | protein tyrosine phosphatase, non- | SEQ ID NO: 1734 |
| | | | | | receptor type 21 | |
| 154 | image: 205314 | Ughs.408312:175 | tp53 | nm_000546 | tumor protein p53 (li-fraumeni | SEQ ID NO: 1735 |
| | | | | | syndrome) | |
| 348 | image: 35072 | Ughs.76152:175 | aqp1 | nm_000385, | aquaporin 1 (channel-forming | SEQ ID NO: 1736 |
| | | | | nm_198098 | integral protein, 28 kda) | SEQ ID NO: 1737 |
| 404 | image: 41452 | Ughs.28491:175 | sat | nm_002970 | spermidine/spermine n1- | SEQ ID NO: 1636 |
| | | | | | acetyltransferase | |
| 412 | image: 42214 | Ughs.192182:175 | syk | nm_003177 | spleen tyrosine kinase | SEQ ID NO: 1738 |
| 416 | image: 430090 | Ughs.355307:175 | tnfrsf7 | nm_001242 | tumor necrosis factor receptor | SEQ ID NO: 1739 |
| | | | | | superfamily, member 7 | |
| 431 | image: 45831 | Ughs.279920:175 | ywhab | nm_003404, | tyrosine 3- | SEQ ID NO: 1740 |
| | | | | nm_139323 | monooxygenase/tryptophan 5- | SEQ ID NO: 1741 |
| | | | | | monooxygenase activation protein, | |
| | | | | | beta polypeptide | |
| 451 | image: 488316 | Ughs.368256:175 | ltbp1 | nm_000627, | latent transforming growth factor | SEQ ID NO: 1742 |
| | | | | nm_206943 | beta binding protein 1 | SEQ ID NO: 1743 |
| 479 | image: 510161 | Ughs.1600:175 | cct5 | nm_012073 | chaperonin containing tcp1, subunit 5 | SEQ ID NO: 1744 |
| | | | | | (epsilon) | |
| 486 | image: 512000 | Ughs.411826:175 | ube2d3 | nm_003340, | ubiquitin-conjugating enzyme e2d 3 | SEQ ID NO: 1745 |
| | | | | nm_181886, | (ubc4/5 homolog, yeast) | SEQ ID NO: 1746 |
| | | | | nm_181887, | | SEQ ID NO: 1747 |
| | | | | nm_181888, | | SEQ ID NO: 1748 |
| | | | | nm_181889, | | SEQ ID NO: 1749 |
| | | | | nm_181890, | | SEQ ID NO: 1750 |
| | | | | nm_181891, | | SEQ ID NO: 1751 |
| | | | | nm_181892, | | SEQ ID NO: 1752 |
| | | | | nm_181893 | | SEQ ID NO: 1753 |
| 498 | image: 530034 | Ughs.544630:175 | | | transcribed locus | |
| 535 | image: 549173 | Ughs.192023:175 | eif3s2 | nm_003757 | eukaryotic translation initiation | SEQ ID NO: 1754 |
| | | | | | factor 3, subunit 2 beta, 36 kda | |
| 622 | image: 79598 | Ughs.194657:175 | cdh1 | nm_004360 | cadherin 1, type 1, e-cadherin | SEQ ID NO: 1755 |
| | | | | | (epithelial) | |

In a further preferred embodiment, the sets for analyzing differential gene expression associated with MSI phenotype can, for example, consist of those mentioned in Table 6:

TABLE 6

| | | | Gene | Reference | | |
|---|---|---|---|---|---|---|
| Set | Clone identifier | Cluster | Symbol | sequences | Title of cluster | SEQ ID Numbers |
| 109 | image: 159885 | ughs.298469:175 | Ace | nm_000789, | angiotensin i converting enzyme | SEQ ID NO: 1731 |
| | | | | nm_152830 | (peptidyl-dipeptidase a) 1 | SEQ ID NO: 1732 |
| | | | | nm_152831 | | SEQ ID NO: 1733 |
| 154 | image: 205314 | ughs.408312:175 | tp53 | Nm_000546 | tumor protein p53 (li-fraumeni | SEQ ID NO: 1735 |
| | | | | | syndrome) | |
| 412 | image: 42214 | ughs.192182:175 | Syk | Nm_003177 | spleen tyrosine kinase | SEQ ID NO: 1738 |
| 486 | image: 512000 | ughs.411826:175 | ube2d3 | nm_003340, | ubiquitin-conjugating enzyme e2d 3 | SEQ ID NO: 1745 |
| | | | | nm_181886 | (ubc4/5 homolog, yeast) | SEQ ID NO: 1746 |
| | | | | nm_181887 | | SEQ ID NO: 1747 |
| | | | | nm_181888 | | SEQ ID NO: 1748 |
| | | | | nm_181889 | | SEQ ID NO: 1749 |
| | | | | nm_181890 | | SEQ ID NO: 1750 |
| | | | | nm_181891 | | SEQ ID NO: 1751 |
| | | | | nm_181892 | | SEQ ID NO: 1752 |
| | | | | nm_181893 | | SEQ ID NO: 1753 |
| 535 | image: 549173 | ughs.192023:175 | eif3s2 | Nm_003757 | eukaryotic translation initiation | SEQ ID NO: 1754 |
| | | | | | factor 3, subunit 2 beta, 36 kda | |
| 622 | image: 79598 | ughs.194657:175 | cdh1 | Nm_004360 | cadherin 1, type 1, e-cadherin | SEQ ID NO: 1755 |
| | | | | | (epithelial) | |

In a further embodiment, the method of the present invention is directed to the analysis of differential gene expression associated with survival and death of patients in colon cancer. In this embodiment, said analysis comprises the detection of the overexpression or the underexpression of a pool of polynucleotide sequences in colon tissues, said pool corresponding to all or part of the polynucleotide sequences, subsequences or complements thereof, selected from each of predefined polynucleotide sequences sets consisting of sets:

2; 3; 5; 7; 8; 10; 12; 14; 20; 22; 23; 26; 28; 32; 33; 35; 36; 41; 42; 44; 47; 50; 51; 60; 61; 63; 64; 70; 73; 74; 81; 92; 93; 95; 106; 115; 118; 120; 121; 123; 129; 130; 132; 133; 137; 145; 148; 149; 160; 161; 162; 163; 183; 187; 188; 195; 199; 200; 202; 206; 209; 211; 213; 214; 217; 219; 222; 228; 229; 230; 233; 234; 238; 245; 246; 247; 250; 257; 269; 271; 274; 275; 276; 282; 283; 284; 285; 289; 291; 292; 296; 302; 303; 304; 312; 314; 318; 323; 327; 333; 334; 335; 336; 337; 339; 340; 341; 342; 344; 345; 347; 350; 351; 356; 359; 361; 362; 363; 364; 367; 370; 373; 374; 378; 380; 381; 382; 383; 384; 387; 389; 402; 403; 408; 411; 414; 418; 420; 428; 430; 433; 435; 439; 444; 446; 447; 449; 456; 457; 458; 460; 461; 465; 473; 478; 482; 484; 489; 490; 491; 494; 497; 501; 502; 504; 510; 514; 516; 520; 523; 528; 529; 530; 536; 537; 538; 539; 540; 548; 551; 556; 561; 562; 570; 571; 580; 581; 582; 584; 586; 590; 591; 593; 594; 596; 603; 607; 609; 612; 615; 620; 624; 625; 628; 635; 639; and 640.

The analysis can comprise at least one of the following steps:
The detection of the overexpression of a pool of polynucleotide sequences in colon tissues, said pool corresponding to all or part of the polynucleotide sequences, subsequences or complements thereof selected from each of predefined polynucleotide sequence sets consisting of sets:

5; 14; 36; 44; 61; 64; 70; 81; 95; 115; 121; 132; 183; 209; 228; 275; 333; 334; 350; 367; 373; 435; 439; 523; 570; 603; and 625.
The detection of the underexpression of a pool of polynucleotide sequences in colon tissues, said pool corresponding to all or part of the polynucleotide sequences, subsequences or complements thereof, selected from each of predefined polynucleotide sequence sets consisting of sets:

2; 3; 7; 8; 10; 12; 20; 22; 23; 26; 28; 32; 33; 35; 41; 42; 47; 50; 51; 60; 63; 73; 74; 92; 93; 106; 118; 120; 123; 129; 130; 133; 137; 145; 148; 149; 160; 161; 162; 163; 187; 188; 195; 199; 200; 202; 206; 211; 213; 214; 217; 219; 222; 229; 230; 233; 234; 238; 245; 246; 247; 250; 257; 269; 271; 274; 276; 282; 283; 284; 285; 289; 291; 292; 296; 302; 303; 304; 312; 314; 318; 323; 327; 335; 336; 337; 339; 340; 341; 342; 344; 345; 347; 351; 356; 359; 361; 362; 363; 364; 370; 374; 378; 380; 381; 382; 383; 384; 387; 389; 402; 403; 408; 411; 414; 418; 420; 428; 430; 433; 444; 446; 447; 449; 456; 457; 458; 460; 461; 465; 473; 478; 482; 484; 489; 490; 491; 494; 497; 501; 502; 504; 510; 514; 516; 520; 528; 529; 530; 536; 537; 538; 539; 540; 548; 551; 556; 561; 562; 571; 580; 581; 582; 584; 586; 590; 591; 593; 594; 596; 607; 609; 612; 615; 620; 624; 628; 635; 639; and 640.

In a preferred embodiment the sets for analyzing differential gene expression associated with the survival and death of patients may for example consist of those mentioned in Table 7:

TABLE 7

| | | | Gene | Reference | | |
|---|---|---|---|---|---|---|
| Set | Clone identifier | cluster | Symbol | sequences | Title of cluster | SEQ ID Numbers |
| 10 | image: 108370 | ughs.366546:175 | map2k2 | nm_030662 | mitogen-activated protein kinase | SEQ ID NO: 1756 |
| | | | | | kinase 2 | |
| 12 | image: 108399 | | | | | |
| 33 | image: 121265 | ughs.181315:175 | ifnar1 | nm_000629 | interferon (alpha, beta and omega) | SEQ ID NO: 1683 |
| | | | | | receptor 1 | |
| 214 | image: 257445 | ughs.77917:175 | uchl3 | nm_006002 | ubiquitin carboxyl-terminal esterase | SEQ ID NO: 1757 |
| | | | | | 13 (ubiquitin thiolesterase) | |
| 217 | image: 258313 | ughs.432170:175 | cox7b | nm_001866 | cytochrome c oxidase subunit viib | SEQ ID NO: 1685 |
| 271 | image: 301119 | ughs.80691:175 | ckmt2 | nm_001825 | creatine kinase, mitochondrial 2 | |
| | | | | | (sarcomeric) | |
| 344 | image: 346610 | ughs.184510:175 | sfn | nm_006142 | stratifin | SEQ ID NO: 1758 |
| 383 | image: 37630 | ughs.300701:175 | mgc8685 | nm_178012 | tubulin, beta polypeptide paralog | SEQ ID NO: 1759 |
| 387 | image: 376755 | ughs.24341:175 | taz | nm_015472 | transcriptional co-activator with pdz- | SEQ ID NO: 1760 |
| | | | | | binding motif (taz) | |
| 414 | image: 428103 | ughs.1311:175 | Cd1c | nm_001765 | cd1c antigen, c polypeptide | SEQ ID NO: 1761 |
| 473 | image: 509588 | ughs.421646:175 | taf12 | nm_005644 | taf12 rna polymerase ii, tata box | SEQ ID NO: 1706 |
| | | | | | binding protein (tbp)-associated | |
| | | | | | factor, 20 kda | |
| 484 | image: 510977 | ughs.173724:175 | ckb | nm_001823 | creatine kinase, brain | SEQ ID NO: 1707 |
| 516 | image: 544885 | ughs.469653:175 | rp15 | nm_000969 | ribosomal protein 15 | SEQ ID NO: 1708 |
| 536 | image: 549178 | ughs.448580:175; | sec611; | nm_007277; | sec6-like 1 (s. cerevisiae); tyrosine 3- | SEQ ID NO: 1762 |
| | | ughs.74405:175 | ywhaq | nm_006826 | monooxygenase/tryptophan 5- | SEQ ID NO: 1763 |
| | | | | | monooxygenase activation protein, | |
| | | | | | theta polypeptide | |
| 561 | image: 611623 | ughs.124979:175; | dj159a19.3; | nm_020462; | hypothetical protein dj159a19.3; | SEQ ID NO: 1764 |
| | | ughs.519765:175 | kiaa1181 | | kiaa1181 protein | |

In a further embodiment the method of the present invention is directed to the analysis or differential gene expression associated with the location of primary colorectal carcinoma in colon cancer. In this embodiment, said analysis comprises the detection of the overexpression or the underexpression of a pool of polynucleotide sequences in colon tissues, said pool corresponding to all or part of the polynucleotide sequences, subsequences or complements thereof, selected in from of predefined polynucleotide sequence sets consisting of sets:

6; 19; 43; 49; 83; 89; 94; 100; 151; 168; 172; 177; 224; 252; 258; 265; 309; 315; 316; 320; 322; 328; 355; 365; 391; 443; 453; 455; 466; 483; 496; 499; 506; 512; 513; 515; 517; 531; 532; 554; 563; 575; 579; 606; 618; and 637.

The analysis can comprise at least one of the following steps:
The detection of the overexpression of a pool of polynucleotide sequences in left-colon tissues, said pool corresponding to all or part of the polynucleotide sequences, subsequences or complements thereof selected from each of predefined polynucleotide sequence sets consisting of sets:

19; 43; 89; 94; 100; 168; 224; 309; 328; 355; 391; 466; 531; 532; 563; and 637.
The detection of the overexpression of a pool of polynucleotide sequences in right-colon tissues, said pool corresponding to all or part of the polynucleotide sequences, subsequences or complements thereof, selected from each of predefined polynucleotide sequence sets consisting of sets:

6; 49; 83; 151; 172; 177; 252; 258; 265; 315; 316; 320; 322; 365; 443; 453; 455; 483; 496; 499; 506; 512; 513; 515; 517; 554; 575; 579; 606; and 618.

In a preferred embodiment, the sets for analyzing differential gene expression associated with the location of the primary colorectal carcinoma can, for example, consist of those mentioned in Table 8:

TABLE 8

| | | | Gene | Reference | | |
|---|---|---|---|---|---|---|
| Set | Clone identifier | cluster | Symbol | sequences | Title of cluster | SEQ ID Numbers |
| 43 | image: 124345 | ughs.77204:175 | cenpf | nm_016343 | centromere protein f, 350/400 ka | SEQ ID NO: 1765 |
| | | | | | (mitosin) | |
| 100 | image: 154335 | ughs.321234:175 | exosc10 | nm_001001998, | exosome component 10 | SEQ ID NO: 1766 |
| | | | | nm_002685 | | SEQ ID NO: 1767 |
| 151 | image: 204653 | ughs.174142:175 | csf1r | nm_005211 | colony stimulating factor 1 receptor, | SEQ ID NO: 1768 |
| | | | | | formerly mcdonough feline sarcoma | |
| | | | | | viral (v-fms) oncogene homolog | |
| 172 | image: 22295 | ughs.343220:175 | crk | nm_005206, | v-crk sarcoma virus ct10 oncogene | SEQ ID NO: 1769 |
| | | | | nm_016823 | homolog (avian) | SEQ ID NO: 1770 |
| 265 | image: 291448 | ughs.95972:175 | silv | nm_006928 | silver homolog (mouse) | SEQ ID NO: 1771 |
| 315 | image: 325641 | ughs.534030:175 | psg5 | nm_002781 | pregnancy specific beta-1- | SEQ ID NO: 1772 |
| | | | | | glycoprotein 5 | |
| 443 | image: 47986 | ughs.149609:175 | itga5 | nm_002205 | integrin, alpha 5 (fibronectin | SEQ ID NO: 1652 |
| | | | | | receptor, alpha polypeptide) | |
| 499 | image: 530037 | ughs.244230:175 | | | full-length cdna clone cs0di056yj24 | |
| | | | | | of placenta cot 25-normalized of | |
| | | | | | homo sapiens (human) | |
| 532 | image: 549065 | ughs.169744:175 | g22p1 | nm_001469 | thyroid autoantigen 70 kda (ku | SEQ ID NO: 1773 |
| | | | | | antigen) | |
| 554 | image: 594120 | ughs.8364:175 | pdk4 | nm_002612 | pyruvate dehydrogenase kinase, | SEQ ID NO: 1774 |
| | | | | | isoenzyme 4 | |

Tables 2 to 8 provide, for each set listed, certain features, some of which are redundant with Table 1 and some of which are additional. For instance, certain reference sequences (“NM_xxxxxx”) in the “Reference Sequences” column of Tables 2 to 8 are supplemental to the sequences mentioned in the “Ref.” column of Table 1. This “Reference Sequences” column provides one or more mRNA references for a specific corresponding gene. These mRNAs, that represent the various splice forms currently identified in the art, are encompassed by the nucleotide sequence sets listed in Tables 2 to 8. Each of these mRNAs can be considered as a marker in the meaning of the present invention. The use of the “NM_xxxxxx” references herein would be clearly understood by a person skilled in the art who is familiar with this type of referencing system. The sequences corresponding to each “NM_xxxxxx” reference (or corresponding splice forms) are available, e.g., in the OMIM and LocusLink databases (NCBI web site) and are incorporated herein by reference. An “NM_xxxxxx” reference is therefore a constant; i.e., it will always designate the same sequence over time and whatever the source (database, printed document, or the like).

Each set described herein comprises sequence(s) mentioned in Table 1 and, in addition, can comprise the “NM_XXXXXX” sequence and splice form(s) thereof mentioned in Tables 2 to 8 for each same set. For example, the sequences that comprise Set 1 are SEQ ID No. 1, 2 (of Table 1) and nm_{—}001747 sequence (of Table 2), including subsequences, or complements thereof, as described previously. In case of redundancy between the “Ref.” column of Table 1 and the “References Sequences” column of Tables 2 to 8 (i.e., if a “NM_XXXXXX” reference sequence corresponds to a SEQ ID sequence already mentioned in “Ref” column of Table 1), only one of these sequences may be considered.

The present invention further relates to a polynucleotide library useful for the molecular characterization of a colon cancer, comprising or corresponding to a pool of polynucleotide sequences which are either overexpressed or underexpressed in one or more of the above-cited tissues (e.g., colon tissue) said pool corresponding to all or part of the polynucleotide sequences (or markers) selected as defined above.

The detection of over or under expression of polynucleotide sequences according to the method of the invention can be carried out by fluorescence in-situ hybridization (FISH) or immuno histochemical (IHC), methods. Such detection can be performed on nucleic acids from a tissue sample, e.g., from one or more of the above-cited tissues, e.g., colorectal tissue sample, or from a tumor cell line.

The invention also relates particularly to a method performed on DNA or cDNA arrays; e.g., DNA or cDNA microarrays.

The detection of over or under expression of polynucleotide sequences according to the method of the invention can also be carried out at the protein level. Such detections are performed on proteins expressed from nucleic acid in one or more of the above-cited tissue samples.

Accordingly, a further method according to the present invention comprises:

a) obtaining a sample comprising proteins from a colorectal tissue sample from a subject; and

b) measuring in said sample obtained in step (a) the level of those proteins encoded by a polynucleotide library according to the invention.

The present invention is useful for detecting, diagnosing, staging, classifying, monitoring, predicting, and/or preventing colorectal cancer. It is particularly useful for predicting clinical outcome of colon cancer and/or predicting occurrence of metastatic relapse and/or determining the stage or aggressiveness of a colorectal disease in at least about 50%, e.g., at least about 55%, at least about 60%, at least about 65%, at least about 70%, at least about 75%, at least about 80%, at least about 85%, at least about 90%, at least about 95%, or about 100% of the subjects. The invention is also useful for selecting a more appropriate dose and/or schedule of chemotherapeutics and/or biopharmaceuticals and/or radiation therapy to circumvent toxicities in a subject.

By “aggressiveness of a colorectal disease” is meant, e.g., cancer growth rate or potential to metastasize; a so-called “aggressive cancer” will grow or metastasize rapidly or significantly affect overall health status and quality of life.

By “predicting clinical outcome” is meant, e.g., the ability for a skilled artisan to classify subjects into at least two classes (good vs. poor prognosis) showing significantly different long-term Metastasis Free Survival (MFS).

In particular, the method of the invention is useful for classifying cell or tissue samples from subjects with histopathological features of colorectal disease, e.g., colon tumor or colon cancer, as samples from subjects having a “poor prognosis” (i.e., metastasis or disease occurred within 5 years since diagnosis) or a “good prognosis” (i.e., metastasis- or disease-free for at least 5 years of follow-up time since diagnosis).

The present invention further relates to a method of assigning a therapeutic regimen to subject with histopathological features of colorectal disease, for example colon cancer, comprising:

a) classifying said subject having a “poor prognosis” or a “good prognosis” on the basis of the method of analysing according to the present invention;

b) assigning said subject a therapeutic regimen, said therapeutic regimen (i) comprising no adjuvant chemotherapy if the subject is lymph node negative and is classified as having a good prognosis, or (ii) comprising chemotherapy if said subject has any other combination of lymph node status and expression profile.

For example, the assigning of a therapeutic regimen can comprise the use of an appropriate dose of irinotecan drug compound. For example, this dose is selected according to the presence or the absence of a polymorphism(s) in a uridine diphosphate glucuronosyltransferase I (UGT1A1) gene promoter of the subject. For example, a polymorphism may be the presence of an abnormal number of (TA) repeats in said UGT1A1 promoter.

More generally, the invention is also useful for selecting appropriate doses and/or schedules of chemotherapeutics and/or (bio)pharmaceuticals, and/or targeted agents, which can include irinotecan, 5-fluorouracil, fluorouracil, levamisole, mitomycin, lomustine, vincristine, oxaliplatin, methotrexate, and anti-thymidilate synthase. Further relevant anti-colorectal cancer agents are known in the art. These agents may administered alone or in combination.

The method for analyzing differential gene expression associated with histopathologic features of colorectal disease according to the present invention, e.g., the method for classifying cell or tissue samples, allows one to achieve high specificity and/or sensitivity levels of at least about 80%, at least about 85%, at least about 90%, at least about 91%, at least about 92%, at least about 93%, at least about 94%, at least about 95%, at least about 96%, at least about 97%, at least about 98%, or at least about 99%.

By “specificity” is meant:
Number of true negative samples×100/(Number of true negative samples+Number of false positive samples)

By “sensitivity” is meant:
Number of true positive samples×100/(Number of true positive samples+Number of false negative samples)

With reference to the figures:

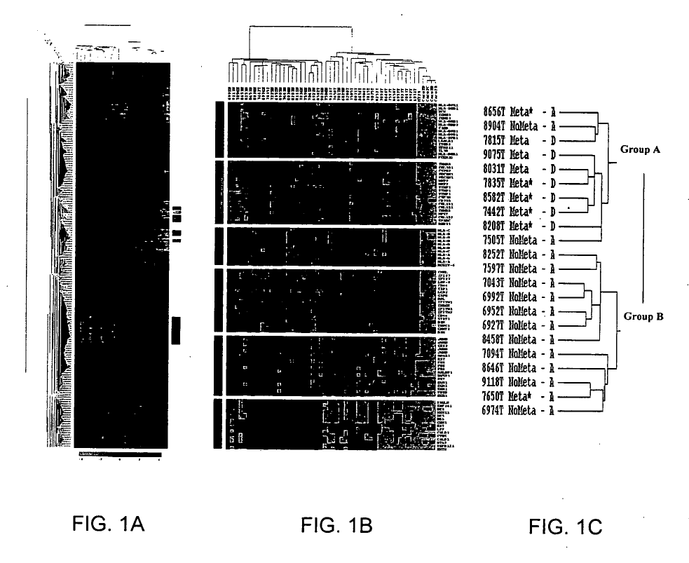

FIG. 1 shows global gene expression profiles in colorectal cancer and non-cancerous samples. 1A—Hierarchical clustering of 50 samples and ˜9,000 cDNA clones based on mRNA expression levels. Each row represents a clone and each column represents a sample. Expression level of each gene in a single sample is relative to its median abundance across all samples and depicted according to a color scale shown at the bottom. Red and green indicate expression levels above and below the median, respectively. The magnitude of deviation from the median is represented by the color saturation. Grey indicates missing data. Dendrogram of samples (above matrix) and genes (to the left of matrix) represent overall similarities in gene expression profiles. For samples, black branches represent normal tissues (n=23), red branches represent cancer tissues (n=22) and purple branches represent cancer cell lines (n=5). Colored bars to the right indicate the locations of 7 gene clusters of interest. These clusters, except the “proliferation cluster” (brown bar), are zoomed in B. 1B—Top panel: dendrogram of samples: tissue samples are designated with numbers followed by N when non-cancerous tissue and T when tumor tissue. Lower panel: expanded view of selected gene clusters named from top to bottom: “MHC class II”, “stromal”, “MHC class I”, “interferon-related”, “early response”, “smooth muscle” and “proliferation”. Genes are referenced by their HUGO abbreviation as used in “Locus Link”. 1C—Dendrogram of samples representing the results of the same hierarchical clustering applied only to the 22 cancer tissue samples. Two groups of samples (A and B) are defined. Sample names and branches highlighted in blue and in red represent patient samples without and with metastatic disease at diagnosis (labelled by *) or during follow-up, respectively. Status of each patient at last follow-up is marked by A (alive) or D (deceased)from CRC.

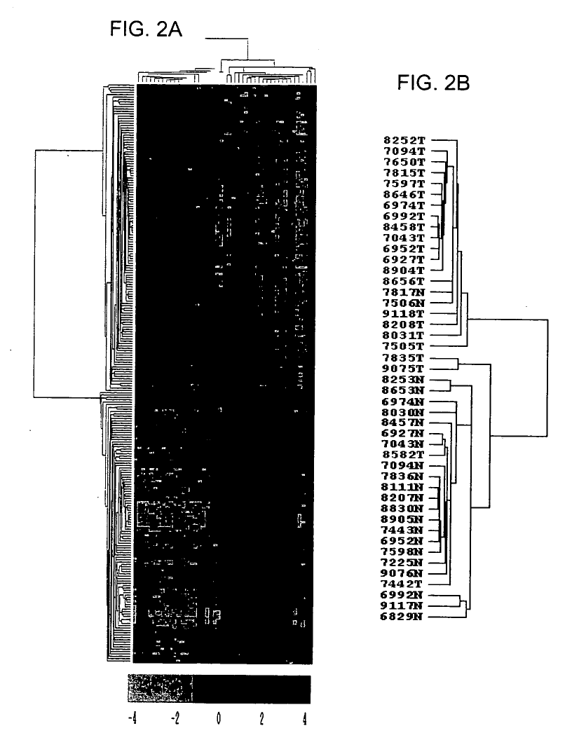

FIG. 2 shows hierarchical classification of tissue samples using genes which discriminate between normal and cancer samples. 2A—Hierarchical clustering of the 45 colon tissue samples using expression levels of the 245 cDNA clones were significantly different between normal and cancer samples. Dendrogram of these samples are magnified in B. 2B—Dendrogram of samples: black branches represent normal tissues (n=23) and red branches represent cancer tissues (n=22).

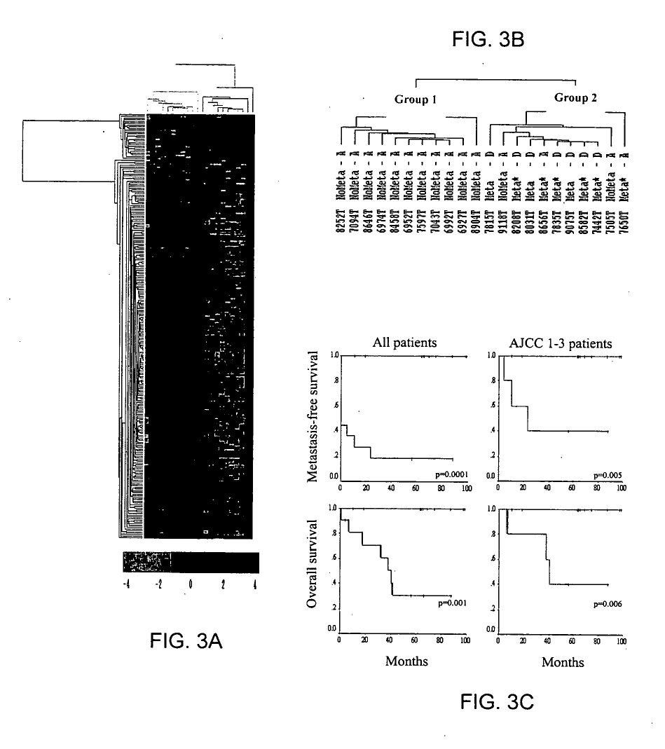

FIG. 3 shows hierarchical classification of CRC tissue samples using genes that discriminate metastatic from non-metastatic samples, correlated with survival. 3A—Hierarchical clustering of the 22 CRC tissue samples based on expression levels of the 244 cDNA clones was significantly different between metastatic and non-metastatic cancer samples. Dendrogram of samples is zoomed in B. 3B—Dendrogram of samples: blue represents samples without metastasis and red represents samples with metastasis at diagnosis (labelled by *) or during follow-up. A means alive at last follow-up and D means dead, from CRC. The analysis delineates 2 groups of tumors, group 1 and group 2. 3C—Kaplan-Meier plots of metastasis-free survival and overall survival of the 2 groups of samples defined by hierarchical clustering for all patients (left, n=22) and AJCC 1-3 patients (right, n=16).

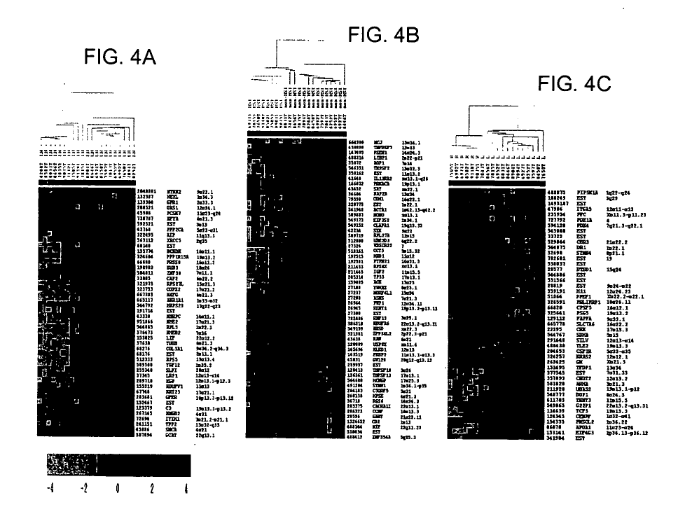

FIG. 4 shows hierarchical classification of CRC tissue samples using discriminator genes selected by supervised analyses based on lymph node status, MSI phenotype and location of tumors. 4A—Hierarchical clustering of the 21 CRC tissue samples based on expression levels of the 46 cDNA clones significantly different between lymph node-positive (LN+, n=5, red branches and names) and lymph node-negative (LN−, n=16, blue branches and names) cancer samples. Each gene is identified by IMAGE cDNA clone number, HUGO abbreviation, and chromosomal location. EST means expressed sequence tag for clones without significant identity to a known gene or protein. 4B—Hierarchical clustering of the 22 CRC tissue samples based on expression levels of the 58 cDNA clones significantly different between MSI+ (MSI, n=8, blue branches and names) and non-MSI (n=14, red branches and names) cancer samples. 4C—Hierarchical clustering of the 22 CRC tissue samples based on expression levels of the 46 cDNA clones was significantly different between cancer samples from right colon (R, n=6, blue branches and names) and left colon (L, n=13, red branches and names).

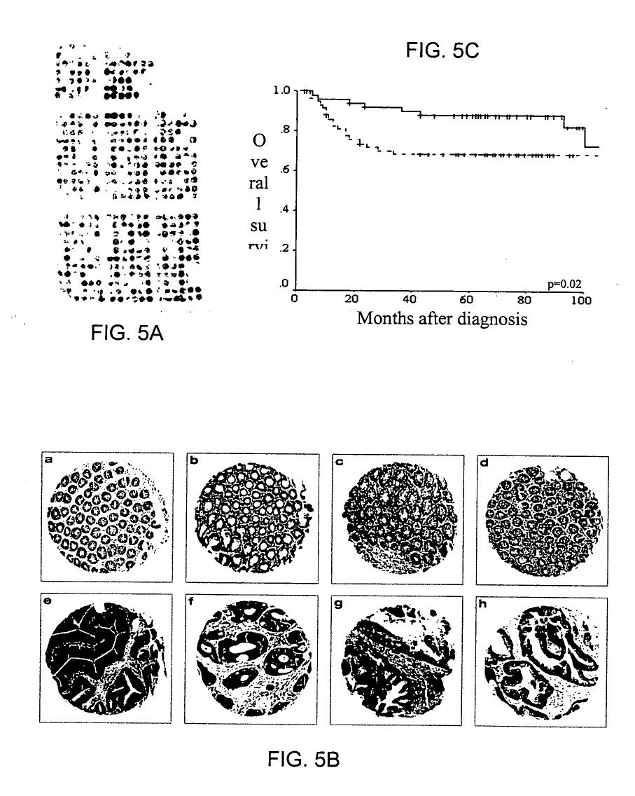

FIG. 5 shows analysis of NM23 protein expression in colorectal tissue samples using tissue microarrays. Protein expression of NM23 was analysed using tissue microarrays containing 190 pairs of cancer samples and corresponding normal mucosa. 5A—Hematoxylin & Eosin staining of a paraffin block section (25x30) from a tissue microarray containing 216 tumors (3×55) and control samples. 5B—Five-μm sections of 0.6 mm core biopsies of cancer colorectal samples stained with anti-NM23 antibody are shown. Sections e and f are from CRC patients without metastasis (strong staining) and Sections g and h are from CRC patients with metastasis (low staining). 5C—Kaplan-Meier plots of overall survival in AJCC1-3 patients according to NM23 protein expression levels. Magnification is 50× in B-E.

### EXAMPLE

The invention will now be illustrated with the following non-limiting examples.

1) Gene expression profiling of CRC and unsupervised classification

The mRNA expression profiles of 50 cancer and non-cancerous colon samples, including 45 clinical tissue samples and 5 cell lines, were determined using DNA microarrays containing ˜9,000 spotted PCR products from known genes and ESTs. Both unsupervised and supervised analyses were performed on all samples following normalization of expression levels.

Unsupervised hierarchical clustering of all samples based on the total gene expression profile was first applied. Results were displayed in a color-coded matrix (FIG. 1A) where samples were ordered on the horizontal axis and genes on the vertical axis on the basis of similarity of their expression profiles. The 50 samples were sorted into two large clusters that extensively differed with respect to normal or cancer type (FIG. 1B, top): 87% were non-cancerous in the left cluster and 87% were cancerous in the right cluster. As expected, the CRC cell lines represented a branch of the “cancer” cluster. Hierarchical clustering also allowed identification of clusters of gene expression corresponding to defined functions or cell types, some of which are indicated by colored bars on the right of FIG. 1A, and which are zoomed in FIG. 1B. Three clusters are overexpressed in tissue samples overall as compared to epithelial cell lines, reflecting the cell heterogeneity of tissues: an “immune cluster” with different subclusters including a MHC class I subcluster that correlated with an interferon-related subcluster, a MHC class II subcluster, which is a “stromal cluster” enriched with genes expressed in stromal cells (COL1A1, COL1A2, COL3A1, MMP2, TIMP1, SPARC, CSPG2, PECAM, INHBA), and a “smooth muscle cluster” (CNN1, CALD1, DES, MYH11, SMTN, TAGL) that was globally overexpressed in normal tissue as compared to cancer tissues. An “early response cluster” included immediate-early genes (JUNB, FOS, EGR1, NR4A1, DUSP1) involved in the human cellular response to environmental stress. Conversely, a very large cluster, defined as a “proliferation cluster”, was generally overexpressed in cell lines as compared to tissues, probably reflecting the proliferation rate difference between cells in culture and tumor tissues. This cluster included PCNA that codes for a proliferation marker used in clinical practice, as well as many genes involved in: glycolysis, such as GAPD, LDHA, ENO1; cell cycle and mitosis, such as CDK4, BUB3, CDKN3, GSPT2; metabolism, such as ALDH3A1, cytochrome C oxidase subunits, and GSTP1, and protein synthesis such as genes coding for ribosomal proteins.

The same clustering algorithm applied only to the 22 CRC clinical samples sorted two groups of tumors (A, 10 patients and B, 12 patients) that differed with respect to AJCC stage and clinical outcome (FIG. 1C). Group A included a high proportion of patients presenting with metastases at diagnosis (AJCC4 stage, 5 out of 10) as compared with group B (1 out of 12). Interestingly, 3 out of 5 “AJCC1-3” patients of group A experienced metastatic relapse after a median duration of 18 months (range, 4 to 88) from diagnosis and died from CRC, while none of the 11 “AJCC1-3” patients of group B relapsed or died after a median follow-up of 69 months (range, 10 to 98). This suggests that patients are at higher risk for metastasis in group A than in group B. To identify particular sets of genes that could better define subgroups of samples, supervised analyses were then conducted.

2) Differential gene expression between normal colon and colon tumors

To identify and rank genes with significant differential expression between cancer (22 samples) and non-cancerous colon tissues (23 samples), a discriminating score (DS) combined with iterative random permutation tests was applied. Two hundred forty-five cDNA clones, 130 of which were overexpressed and 115 were underexpressed in cancer samples, were identified. These clones corresponded to 237 unique sequences that represented 191 different known genes and 46 ESTs. The function of the known genes, as given in the OMIM and LocusLink databases (NCBI web site), are listed in Table. 1 above. Samples were then reclustered on the basis of these genes (FIG. 2), with a good resulting discrimination between normal and cancer samples: in the left branch 90% of samples were cancerous, while in the large right branch 92% were normal.

3) Differential gene expression within CRC tissue samples

A supervised approach was applied to the 22 cancer tissue samples by comparing tumor subgroups defined by relevant histoclinical parameters.

3.a) Genes associated with visceral metastases

The occurrence of metastasis is the leading cause of death in patients with CRC. Accurate predictors of metastasis are needed to determine therapeutic strategies and improve survival. Two hundred forty-four cDNA clones, corresponding to 235 unique sequences representing 194 characterized genes and 41 ESTs, were identified that discriminated between primary tumor samples collected from patients with and without metastasis at time of diagnosis or during follow-up. Among these clones, 219 were underexpressed and 25 were overexpressed in metastatic samples as compared to non-metastatic samples. Hierarchical clustering of samples based on expression of these selected genes (FIGS. 3A-B) successfully classified patients according to outcome, with only two non-metastatic samples misplaced in the group 2. Significantly, differences of survival between the two groups were statistically significant (FIG. 3C). The 5-year MFS (Metastatic Free Survival) and OS (Overall Survival) were 100% for group 1 (n=11) and 18% and 30%, respectively, for group 2 (n=11) (p=0.0001 and p=0.001). MFS and OS were 100% for group 1 (n=11) and 40% for the group 2 (n=5) when only patients without metastatic disease at time of diagnosis (AJCC1-3 stage) were considered (p=0.005 and p=0.006, respectively). Finally, MFS and OS were 100% for group 1 (n=10) and 50% for the group 2 (n=4) when only AJCC1-2 patients (no metastatic disease and node-negative tumor at time of diagnosis) were considered (p=0.019 and p=0.022, respectively).

3.b) Genes associated with lymph node metastases

Pathological lymph node involvement at diagnosis is a strong prognostic parameter in CRC. Its determination relies on surgical dissection, which currently requires biopsy of individual lymph nodes. Surgical lymph-node biopsy has major disadvantages, such as patient discomfort and the fact that metastases, particularly micrometastases, are often missed by surgical biopsy. Lymph node involvement is dependent on the heterogenous expression, and complex interaction(s) of these genes, to promote metastatic invasion and clinical outcome. Large-scale expression analyses provide a solution to identify these genes and the complexity of their interactions to drive tumorigenesis and metastatic invasion, as reported for breast or gastric cancers.

Forty-six cDNA clones (41 known genes and 5 ESTs) were identified as significantly differentially expressed between tumors with (n=5) and without (n=16) lymph node metastasis. Reclustering based on these 46 genes correctly separated node-positive from node-negative samples (FIG. 3A). The two samples (9075T and 7442T) that, among all node-negative cases, had expression patterns more closely related to node-positive samples, displayed metastatic disease at time of diagnosis (7442T) and 23 months after surgery (9075T), corroborating the predictions based on molecular signature.

3.c) Genes associated with MSI phenotype and with location of cancer

To obtain additional insights in colorectal oncogenesis, differential gene expression between MSI+(n=8) and non-MSI (n=14) tumors and between tumors from right colon (n=6) and left colon (n=13) were analyzed.

Fifty-eight cDNA clones (representing 51 known genes and 5 ESTs) with significant differential expression between MSI+ and non-MSI tumors were identified. The discriminator potential of these clones was confirmed by hierarchical classification of samples based on their expression levels, even if some MSI+ tumors displayed an intermediate expression profile (FIG. 4B). Similarly, classification of 19 samples (excluding transverse colon tumors), based on the expression of 46 cDNA genes (35 known genes and 11 ESTs) differentially expressed between right and left colon cancers, correctly sorted samples from the right or left colon (FIG. 4C). Such discrimination agreed with the existence of two distinct categories of CRC according to the location of tumor

3.d) Immunohistochemistry on tissue microarrays.

The protein expression levels of the most significant discriminatory genes identified by supervised analyses on TMA's containing 190 pairs of cancer samples and corresponding normal mucosa were measured. Use of TMA allowed the measurement of the expression levels simultaneously and in identical conditions. IHC results using an anti-NM23 antibody (which detects both NMEI and NME2 proteins)are shown in FIG. 5. Consistent with DNA microarray results, NM23 was significantly overexpressed in cancer samples as compared to non-cancerous samples (p=5.6×10⁻⁶, Fisher exact test), and was significantly down-regulated in tumors with metastasis (cut-off was the median value) compared to tumors without metastasis (p=0.04, Fisher exact test). The 5-year MFS was 68% for negative and 88% for positive samples when considering the 111 AJCC1-3 patients with available IHC data (p=0.02, log-rank test). Conversely, no such correlation, identified using DNA microarrays, was found for the protein expression levels of prohibitin and decorin.

4) Discussion

DNA microarray-based gene expression profiling is a promising approach to investigate the molecular complexity of cancer. To date, CRC studies have not directly addressed the issue of prognosis or MSI phenotype. Fifty cancer and non-cancerous colon tissue samples was profiled and expression profiles were correlated with histoclinical parameters of disease, including survival, using both unsupervised and supervised analyses.

4a) Unsupervised analysis

Global gene expression profile revealed extensive transcriptional heterogeneity between samples, notably cancer samples. It was to some extent already able to distinguish clinically relevant subgroups of samples: normal versus cancer tissues as previously reported, notably for CRC, and good versus poor prognosis tumors. Such global classification is usually imperfect because of the excessive noise generated by large gene sets that mask the identification of signicant discriminatory genes (such as clinical outcome) governed by a smaller set. Importantly, described global approach allows identification of discrete expression patterns to define clinical useful classification among patients with CRC: for example, several gene clusters that correspond to cell types (stroma, smooth muscle, MHC class I and II) or function (interferon-related, immediate-early response and proliferation) that have been reported in previous studies were identified; hence the validity of the present data consistent with putative biologic function.

4b) Supervised analyses

To identify smaller sets of discriminator genes that may improve classification of samples and facilitate translation in clinical practice, supervised statistical analyses were done, based on predefined groups of samples.

i) Comparison of normal vs cancer samples.

A total of 245 discriminator cDNA clones (3%) were significantly differentially expressed between normal and cancer samples. This ratio is in agreement with those reported in the literature. Comparison with lists of discriminator genes previously identified in CRC using DNA microarrays revealed many common genes, further underlying the validity of the present data. For example, CA4, CHGA, CNN1, MYH11, FCGBP, KCNMB1, SST were down-regulated, whereas CA3, CCT4, EIF3S6 or EEF1A1, IFITM1, CSE1L, NME1 or RAN were up-regulated in cancer samples. Beyond these common genes, many additional genes to improve the accuracy of previously described predictive signatures were identified.

Among the underexpressed genes in cancer samples were genes encoding cytokines (IL10RA, IL1RN, IL2RB), proteins involved in lipid metabolism (LPP, LIAS, LRP2, MGLL), signal transducers (PLCD1, PLCG2, mTOR/FRAP1), transcription factors such as RELA, and known or putative tumor suppressor genes (TSG). CTCF encodes a transcriptional repressor of MYC and is located in 16q22.1, a chromosomal region frequently deleted in breast and prostate tumors; IRF1, a transcriptional activator of genes induced by cytokines and growth factors, regulates apoptosis and cell proliferation and is frequently deficient in human cancers. The underexpression of GSN (gelsolin), combined with that of PRKCB1 (protein kinase C, beta 1), may lead to decreased activation of PKCs involved in phospholipid signalling pathways that inhibit cell proliferation and tumorigenicity.

The top-ranked gene overexpressed in cancer samples was GNB2L1 (also named RACK1) that encodes a beta polypeptide 2-like 1 of a guanine nucleotide binding protein (G protein) involved in signal transduction and activation of PKC. It also interacts with IGF1R, shown to play a pivotal role in colorectal oncogenesis; this interaction may regulate IGF1-mediated AKT activation and protection from cell death as well as IGF1-dependent integrin signalling and promote cell extravasion and contact with extracellular matrix (ECM). Other genes have already been reported as up-regulated in other types of cancer: they encode SNRPs and SOX transcription factors (SNRPC, SNRPE, SOX4, SOX9), components of ECM, and molecules involved in vascular and extracellular remodelling (COL5A1, P4HA1, MMP13, LAMR1). BZRP, that codes for the peripheral benzodiazepine receptor, cell cycle genes (CCNB2, CDK2), and SAT, involved in polyamine metabolism were also identified. Consistent with previous reports, we identified the overexpression in cancer samples of SERPINB5 and NME1, encoding two potential TSGs. Overexpression of NME1 combined with underexpression of CTCF interacts to induce overexpression of the MYC oncogene, an important modulator of WNT/APC signalling shown to play an important role in the development of CRC. Other up-regulated genes, and potential therapeutic targets, include kinases (PTK2, STK6, NTRK2), the cell-surface protein CD9, and three genes encoding integrins ITGA2, ITGAL and ITGB3. The integrin pathway was further affected with variations in the expression of genes encoding PTK2, TGFB1I1/HIC5 (a PTK2 interactor), and integrin-linked kinase ILK. Agrawal et al. previously identified osteopontin, an integrin-binding protein as a marker of CRC progression. SPP1 that codes for osteopontin, as well as CXCL1 which codes for GRO1 oncogene or CDK4, were not in the present stringent list of discriminator genes, although overexpressed in cancer samples with a fold-change greater or equal to 2.

Discriminator genes were associated with many cell structures, processes and functions, including general metabolism (the most abundant category), cell cycle, proliferation, apoptosis, adhesion, cytoskeletal remodelling, signal transduction, transcription, translation, RNA and protein processing, immune system and others. Up- and down-regulated genes were rather equally distributed with respect to these functions, except for those coding for kinases and for proteins involved in extracellular matrix remodelling, metabolism, RNA and protein processing (translation, ribosomal proteins and chaperonins), which were overexpressed in cancer samples as compared to normal samples. This phenomenon, already reported, is likely to be related to increased metabolism and cell proliferation in cancer cells.

Analysis of chromosomal location point to two interesting regions. Six genes up-regulated in cancer (STK6, UBE2C, PFDN4, RPS21, CSE1L, SLPI) were located in 20q13, a chromosomal region often amplified in cancer; their overexpression might be a consequence of gene amplification. This has already been observed by others, although not all genes of the region are affected transcriptionally. Conversely, six genes (TJP3, INSR, ELAVL1, MAP2K7, CNN1, NR2F6) down-regulated in cancer samples were located in 19p13.1-p13.3, already known to harbour several potential TSG such as APC2, STK11 or MCC2.

ii) Expression profiles and clinical outcome

All subjects, some of them presenting with metastasis at diagnosis, had received standard treatment. Significantly, the described method for global hierarchical clustering from subjects with non-metastatic tumors that clustered with metastatic cases eventually developed metastasis and died during follow-up. Supervised analysis further improved the prognostic classification by identifying 194 known genes and 41 ESTs that well discriminated between samples without or with metastasis at diagnosis or during follow-up. This is the first report that suggests a potential prognostic role of gene expression profiling in CRC. The significance of the prognostic classification made by AJCC stage and by expression levels of the present discriminator gene sets were compared. Classification based on AJCC stage (AJCC1-2 tumors, n=14, vs AJCC3-4 tumors, n=8) was significant (p=0.001; Kaplan-Meier survival analysis, log-rank test), but less than that made by expression profiles (Fisher's exact test, p=0.05 vs p=0.003). Significantly, the prognostic impact of our gene set was also confirmed when applied to patients without metastasis at diagnosis as well as to patients without metastasis and lymph node invasion.

In addition, the functional identities of the discriminator genes provided insight into the underlying molecular mechanism that drive the metastatic process, and contributed to the identification of potential novel therapeutic targets. For example, known genes that were down-regulated in metastatic tumors were DSC2, encoding desmocollin 2, a desmosomal and hemi-desmosomal adhesion molecule of the cadherin family, HPN, coding for hepsin, a transmembrane serine protease the favorable prognostic role of which has been recently highlighted in prostate cancer by studies using DNA and/or tissue microarrays. Decorin is a small leucine-rich proteoglycan abundant in ECM that negatively controls growth of colon cancer cells and angiogenesis. Low levels of mRNA have been associated with a worse prognosis in breast carcinomas. NME1 and NME2 were underexpressed in patients that developed metastasis, consistent with previous reports that these genes interacted to suppress metastasis. Prohibitin is a mitochondrial protein thought to be a negative regulator of cell proliferation and may be a TSG. Transcription of genes encoding mitochondrial proteins has been shown to be decreased during progression of CRC. This was confirmed in the present study, since all discriminator genes involved in mitochondrial metabolism were down-regulated in metastatic tumors (ATP5C1, BCKDK, CABC1, CKMT2, COX5B, COX6B, COX7A2, COX7A2L, COX7C, HSPA9B, LRIG1, MDH1, NDUFA1, NDUFA4, NDUFA6, NDUFA9, NDUFV1, SCO1, UQCR). Surprisingly, although increased protein synthesis is classically associated with oncogenic transformation, we found many genes coding for ribosomal proteins (RPL5, RPL6, RPL15, RPL29, RPL31, RPL39) were found that were down-regulated in metastatic tumors. The SMAD1/AMDH1 gene codes for a transmitter of TGFalpha signalling, which exerts a number of regulatory effects on colon cells and is involved in the metastatic process. The most significantly overexpressed genes in metastatic tumors were PCSK7, which codes for the proprotein convertase subtilisin/kexin type 7. Proprotein convertases (PCs) process latent precursor proteins into their biologically active products, including protein tyrosine phosphatases, growth factors and their receptors, and enzymes like matrix metalloproteases (MMPs), that may confer on them a functional role in the tumor cell invasion and tumor progression. Other up-regulated genes encoded various signalling proteins including PRAME, an interactor of the cytoskeleton-regulator paxillin, IQGAP1, a negative regulator of the E-cadherin/catenin complex-based cell-cell adhesion, LTPB4, a structural component of connective tissue microfibrils and local regulator of TGFβ tissue deposition and signalling, IGF1R, a transmembrane tyrosine kinase receptor, and DSG1, another desmosomal cadherin-like protein. The incorrect balance between the various desmosomal cadherins has been shown to facilitate separation of epithelial from the ECM and metastasis. IGF1R has been recently shown as involved in metastases of CRC by preventing apoptosis, enhancing cell proliferation, and inducing angiogenesis. Several genes located on the long arm of chromosome 15 were down-regulated in metastatic samples.

iii) Expression profiles and lymph node metastasis

Although nodal metastasis is currently the standard clinical method to predict patient prognosis, there is clear consensus that an improved diagnostic is required to accurately predict survival for patients with CRC. However, approximately one-third of node-negative CRC recur, possibly due to understaging and inadequate pathological examination of lymph nodes. Statistical models suggest that the mean number of nodes currently identified in patients is much too low to correctly classify nodal status. Expression profiles defined in primary tumors could help predict the presence of lymph node metastasis, as recently reported. Forty-six genes and ESTs were identified as discriminators between node-positive and node-negative tumors. Since lymph node status and metastatic relapse are correlated events, this invention includes the identification of novel genes that discriminate between tumors with or without metastasis.

For example, OAS1 and NTRK2 were overexpressed in node-positive tumors. NTRK2 encodes a neurotrophic tyrosine kinase, and aberrant mutation of NTRK2 has recently been shown to play a role in the metastastic process. OAS1 encodes the 2′,5′-oligoadenylate synthetase 1; the 2-5A system has been implicated in the control of cell growth, differentiation, and apoptosis. High levels of activity have been reported in individuals with disseminated cancer, and a recent study found overexpression of OAS1 mRNA in node-positive breast cancers. Conversely, MGP, PRSS8 and NME2 were down-regulated in node-positive tumors. MGP encodes the matrix G1a protein, the loss of expression of which has been associated with lymph node metastasis in urogenital tumors. The prostasin serine protease, encoded by PRSS8, is a potential invasion suppressor, and down-regulation of PRSS8 expression may contribute to invasiveness and metastatic potential. The present list of 46 discriminator clones also included additional genes, reflecting the non-perfect correlation between lymph node metastasis and visceral metastasis and the involvement of different underlying biological processes.

Among genes underexpressed in node-positive tumors were BUB3, TPP2 and ITIH1. BUB3 codes for a mitotic-spindle checkpoint protein that interacts with the APC protein to regulate chromosome segregation during cell division. Defects in mitotic checkpoints, including mutations of BUB1, have been associated with CRC and BUB genes (BUB1 and BUB1B) are underexpressed in highly metastatic colon cell lines. TPP2, encodes tripeptidyl peptidase II, a high molecular mass serine exopeptidase that may play a functional role by degrading peptides involved in invasive and metastatic potential as recently reported for another peptidyl peptidase DPP4. ITIH 1, encodes a heavy chain of proteins of the ITI family, that inhibits the metastatic spreading of H460M large cell lung carcinoma lines by increasing cell attachment.

iv) Expression profiles and MSI phenotype

Without wishing to be bound by any theory, it is believed that there are at least two distinct pathways of oncogenesis in sporadic CRC. Fifteen per cent of tumors present the MSI phenotype, which is related to the inactivation of MMR genes, principally MSH2 and MLH1. The genetically unstable tumor cells accumulate somatic clonal mutations in their genome, which may disturb mRNA expression or degradation of specific transcripts. Conversely, 85% of sporadic tumors are associated with a non-MSI (or MSS) phenotype; they are characterized by chromosome instability and loss of genomic material that may count for the loss of expression of specific alleles. MSI+ tumors are frequently diploid, located in the proximal colon, and may be associated with better prognosis and response to chemotherapy. Reliable distinction between MSI+ and non-MSI phenotypes, currently based on molecular approaches, remains problematic and difficult to assess/confirm in the clinical setting; largely due to the number and heterogeniety of genes involved, absence of easily identifiable mutationional hot-spots, and epigenetic inactivation. Other methods are being tested such as IHC assessment of MSH2 and MLH1

Although the underlying molecular mechanisms of MSI+ and non-MSI colorectal oncogenesis remain unclear, it appears that these two phenotypes represent different molecular entities that could translate into distinct gene expression profiles useful in clinical practice as new diagnostic markers and/or tests. The present supervised analysis of MSI+ and non-MSI CRC clinical samples showed 58 differentially expressed clones. It is of note that arrayed MMR genes (MSH2, MSH3, MLH1, MLH3, PMS1 and PMS2) were not among these discriminator genes. As reported for cell lines, several of these deregulated genes are involved in cell cycle control, mitosis, transcription and/or chromatin structure (RAN, PTPN21, TP53, MORF4L1, ZFP36L2, PSEN1, IGF2, ASNS, RPS4X, CCNF, ZNF354A). The top down-regulated gene in MSI+ tumors was EIF3S2, that encodes the eukaryotic translation initiation factor 3, and subunit 2β, also known as TRIP1 (TGFalpha receptor-interacting protein 1). TRIP1 specifically associates with TGFBRII, a serine/threonine kinase receptor frequently inactivated by mutation and down-regulated in MSI+ tumors.

v) Validation studies

Many different cell processes are aberantly modulated during colorectal oncogenesis. Genes involved in adhesion processes are affected in metastasis. Genes known to be affected in oncogenesis, such as MMR genes, do not discriminate tumor subgroups. DNA microarray data could prove rapidly useful in clinical practice and design of new therapeutic options. The described DNA micro-array approach may be ideally suited to elucidate the complex and heterogeneous processes that drive CRC progression in individual patients, significantly improve clinical treatment of CRC, and optimize the use of novel therapeutic options. Discriminator genes represent potential new diagnostic and prognostic markers and/or therapeutic targets, and deserve further investigation in larger series of subjects. Novel markers of potentially differentially expressed molecules were identified using IHC on TMA containing 190 pairs of cancer samples and corresponding normal mucosa. TMA confirmed the correlations between NM23 expression level and two clinical parameters: non-cancerous or cancer status and survival of patients. Expression was higher in cancer samples, and low expression was significantly associated with a shorter MFS. Such correlation has been described in a variety of malignant tumors, including breast, ovarian, lung or gastric cancers as well as melanoma. However, this correlation remains controversial in CRC, with positive and negative reports. The present invention allowed measurement of the expression levels simultaneously and under highly standardized conditions for all the 190 CRC samples, representing one of the largest series of CRC samples tested for NM23 IHC. 0 As previously described, correlation between protein and mRNA levels would not be expected in all cases. This was the case for Decorin and Prohibitin.

vi) Conclusion.

The data presented in this nonlimiting Examples section shows that mRNA expression profiling of CRC using DNA microarrays provides for identification of clinically relevant tumor subgroups, defined upon combined expression of genes. The genes delineated in this invention can contribute to the understanding of CRC development and progression, and may lead to improved and new diagnostic and/or prognostic markers, identify new molecular targets for novel anticancer drugs, and may also lead to significant improvements in CRC management.

V—Materials and Methods used in the above Examples

1) Colorectal cancer patients and samples

A total of 50 samples including 45 tissue samples and 5 cell lines were profiled using DNA microarrays. The 45 colon tissue samples were obtained from 26 unselected patients with sporadic colorectal adenocarcinoma who underwent surgery at the Institut Paoli-Calmettes (Marseille, France) between 1990 and 1998. Samples were macrodissected by pathologists, and frozen within 30 min of removal in liquid nitrogen for molecular analyses. All tumor samples contained more than 50% tumor cells. The 45 samples included 22 cancer samples and 23 normal samples divided into 19 tumor-normal pairs (based on availability of a sample of the corresponding normal colonic mucosa), 3 tumors and 4 normal specimens provided from different patients. All tumor sections and medical records were de novo reviewed prior to analysis. MSI phenotype of 22 cancer samples was determined by PCR amplification using BAT-25 and BAT-26 oligonucleotide primers, and by IHC using anti-MSH2 and MLH1 antibodies. BAT-25 and BAT-26 are mononucleotide repeat microsatellites: a polyA²⁶ sequence located in the fifth intron of MSH2 for BAT-26, and located in an intron of the KIT gene for BAT-25. Tumors with alterations in both BAT markers were classified as MSI+. No attempt was made to further classify tumors into MSI-high and MSI-low phenotype. Main characteristics of patients and tumors are listed in Table 9. After colonic surgery, subjects were treated (delivery of chemotherapy or not) according to standard guidelines. After completion of therapy, subjects were evaluated at 3-month intervals for the first 2 years and at 6-month intervals thereafter. Search for metastatic relapse included clinical examination and blood tests completed by yearly chest X-ray and liver ultrasound and/or CT scan.

Five samples were represented by 2 different sporadic colon cancer cell lines with chromosomal instability phenotype, Caco2 and HT29. Three samples represented Caco2 in a differentiated state (named Caco2A, 2B and 2C)—i.e. at confluence (C), at C+10 days, at C+21 days—and one sample represented undifferentiated Caco2 (named Caco2D). Cell lines were obtained from the American Type Culture Collection and grown as recommended.

TABLE 9
Characteristics of cancer samples profiled using DNA microarrays

| | | | | | | | | MSI | | Outcome |
|---|---|---|---|---|---|---|---|---|---|---|
| Patient | Sex | Age | Location | Grade | pT UICC | pN UICC | AJCC Stage | status | Treatment | (months) |
| 7650 | M | 74 | descending colon | G | pT3 | pN1 | 4 (liver) | MSI | pS + pCT | AWC 4 |
| 8582 | F | 80 | ascending colon | P | pT3 | pN3 | 4 (liver) | MSI | pS | D 1 |
| 7442 | M | 64 | transverse colon | G | pT3 | pN1 | 4 (liver) | MSS | pS + pCT | D 32 |
| 8208 | M | 40 | transverse colon | M | pT3 | pN2 | 4 (liver) | MSS | cS + adj CT | D 41 |
| 7835 | F | 72 | transverse colon | G | pT3 | pN3 | 4 (liver) | MSS | pS + pCT | D 17 |
| 8656 | F | 57 | descending colon | G | pT3 | pN2 | 4 (liver) | MSS | cS + adj CT | AWC 66 |
| 8031 | F | 46 | descending colon | G | pT3 | pN2 | 3 | MSS | cS + adj CT | MR 4 - D 7 |
| 6927 | M | 71 | descending colon | G | pT3 | NA | NA | MSS | cS + adj CT | NED 10 |
| 9118 | F | 75 | ascending colon | G | pT3 | pN1 | 2 | MSI | cS + adj CT | NED 56 |
| 8904 | M | 80 | descending colon | G | pT3 | pN1 | 2 | MSI | cS | NED 18 |
| 6974 | M | 68 | ascending colon | P | pT3 | pN1 | 2 | MSI | cS + adj CT | NED 97 |
| 8646 | M | 74 | descending colon | G | pT3 | pN1 | 2 | MSS | cS | NED 63 |
| 8458 | M | 56 | descending colon | G | pT3 | pN1 | 2 | MSS | cS + adj CT | NED 69 |
| 6992 | F | 65 | ascending colon | G | pT3 | pN1 | 2 | MSS | cS + adj CT | NED 98 |
| 7094 | F | 87 | descending colon | G | pT3 | pN1 | 2 | MSS | cS | NED 64 |
| 8252 | F | 54 | rectum | G | pT4 | pN1 | 2 | MSS | cS + adj CT | NED 74 |
| 9075 | F | 45 | ascending colon | G | pT2 | pN1 | 1 | MSI | cS | MR23 - D38 |
| 7505 | M | 71 | ascending colon | G | pT1 | pN1 | 1 | MSI | cS | NED 88 |
| 7043 | M | 70 | descending colon | G | pT2 | pN1 | 1 | MSS | cS | NED 97 |
| 6952 | M | 58 | descending colon | G | pT2 | pN1 | 1 | MSS | cS | NED 65 |
| 7597 | F | 72 | rectum | G | pT2 | pN1 | 1 | MSS | cS | NED 87 |
| 7815 | M | 63 | rectum | G | pT2 | pN1 | 1 | MSI | cS | MR 10 - D 40 |

For the IHC study on Tissue Micro Array (TMA), a consecutive series of 191 sporadic CRC patients (including the 26 cases studied by DNA microarrays) treated between 1990 and 1998 at the Institut Paoli-Calmettes was selected. The study included 98 men and 92 women. The median age of patients at diagnosis was 64 years, (range, 29 to 97 years). In 58% of the cases, tumors were located in the distal part of the large bowel or sigmoid, 29% in the proximal part, and 13% in the rectum.

TABLE 10
Characteristics of cancer samples profiled using tissue microarrays.

| Characteristics | All patients (n = 191) |
|---|---|
| Sex (M/F) | 99/92 |
| Median age, years (range) | 64 (29-97) |
| Location of tumor | |
| ascending colon | 47 |
| transverse colon | 9 |
| descending colon | 110 |
| rectum | 21 |
| na | 4 |
| Grade | |
| good | 127 |
| poor | 50 |
| na | 14 |
| pT UICC | |
| 1 | 16 |
| 2 | 21 |
| 3 | 127 |
| 4 | 27 |
| pN UICC | |
| 1 | 88 |
| 2 | 48 |
| 3 | 54 |
| Na | 1 |
| Vascular invasion | |
| no | 115 |
| yes | 68 |
| na | 8 |
| AJCC stage* | |
| 1 | 29 |
| 2 | 51 |
| 3 | 43 |
| 4 | 68 |
| Surgery | 191 |
| curative/palliative | 131/59 |
| na | 1 |
| Chemotherapy | 109 |
| adjuvant/palliative | 60/49 |
| no chemotherapy | 80 |
| na | 2 |
| Median follow-up, months (range) | 74 (2, 133) |
| Metastatic evolution | 95 |
| metastatic relapse* | 27 |
| progression** | 68 |
| Death from CRC | 90 |

Legend:
M, male;
F, female;
na, not available;
pT, pathological staging of primary tumor;
UICC, International Union Against Cancer;
pN, pathological staging of regional lymph nodes;
AJCC, American Joint Committee on Cancer;
*AJCC1-3 patients;
**AJCC4 patients;
CRC, colorectal cancer.

2) RNA extraction

Total RNA was extracted from frozen tumor samples by using standard guanadinium isothiocynanate and cesium chloride gradient techniques. RNA integrity was controlled by denaturing formaldehyde agarose gel electrophoresis and 28-S Northern blots before labelling.

3) DNA microarray preparation

Gene expression analyses were performed with home-made Nylon microarrays containing 8,074 spotted cDNA clones, representing 7,874 IMAGE human cDNA clones and 200 control clones. According to the 155 Unigene release, the IMAGE clones were divided into 6,664 genes and 1,210 ESTs. All clones were PCR-amplified in 96-well microtiter plates (200 μl). Amplification products were desiccated and resuspended in 50 μl of distilled water. They were then spotted as previously described onto Hybond-N+2×7 cm² membranes (Amersham) adhered to glass slides, using a 64-pin print head on a MicroGridII microarrayer (Apogent Discoveries, Cambridge, England). All membranes used in this study belonged to the same batch.

4) DNA microarray hybridizations

Microarrays were hybridized with ³³P-labeled probes: first with an oligonucleotide sequence common to all spotted PCR products (called “vector hybridization” to precisely determine the amount of target DNA accessible to hybridisation in each spot) and then, after stripping, with complex probes made from 2 μg of retrotranscribed total RNA. Probe preparations, hybridizations and washes were done as previously described and available from the website maintained by TAGC ERM206 (INSERM) under the heading “Materials and Methods, ” the entire disclosure of which is herein incorporated by reference. After the washing steps, arrays were exposed to phosphor-imaging plates that were then scanned with a FUJI BAS 5000 machine (25 μm resolution). Hybridization signals were quantified using ArrayGauge software (Fuji Ltd, Tokyo, Japan).

5) Data analysis

Signal intensities were normalized for the amount of spotted DNA and the variability of experimental conditions (FB HMG99). Complex probe intensity of each spot (C) was first corrected (C/V) for the amount of target DNA accessible to hybridization as measured using vector hybridisation (V). When V intensity of a spot was too weak on a microarray, the corresponding cDNA clone was not considered for this experiment. Then, to minimize experimental differences between different complex probe hybridizations, C/V values from each hybridization were divided by the corresponding median value of C/V.

Unsupervised hierarchical clustering analysis then allowed the investigation of relationships between samples and between genes. This analysis was applied to data log-transformed and median-centred on genes using the Cluster and TreeView program (average linkage clustering using Pearson correlation as similarity metric). Supervised analysis was also used to identify and rank genes that distinguished between two subgroups of samples defined by an interesting histoclinical parameter. A discriminating score (DS) was calculated for each gene as DS=(M1−M2)/(S1+S2), where M1 and S1 respectively represent mean and standard deviation of expression levels of the gene in subgroup 1, and M2 and S2 in subgroup 2. Confidence levels were estimated by bootstrap resampling.

Statistical analyses were done using the SPSS software (version 10.0.5). Metastasis-free survival (MFS) and overall survival (OS) were measured from diagnosis until, respectively, the date of the first distant metastasis and the date of death from CRC. Survivals were estimated with the Kaplan-Meier method and compared between groups with the Log-Rank test. Data concerning patients without metastatic relapse or death at last follow-up were censored, as well as deaths from other causes. A p-value <0.05 was considered significant.

6) Tissue microarrays (TMA) construction

The technique of TMA allowed the analysis of tumors and their respective normal mucosa simultaneously and under identical experimental conditions for the 190 subjects. TMA were prepared as described above, with slight modifications. For each sample, three representative sample areas were carefully selected from a hematoxylin-eosin stained section of a donor block. Core cylinders with a diameter of 0.6 mm each were punched from each of these areas and deposited into three separate recipient paraffin blocks, using a specific arraying device (Beecher Instruments, Silver Spring, Md.). In addition to pairs of tumor and normal mucosa, the recipient block also received control tissue (small intestine, adenomas) and cell lines pellets. Five-μm sections of the resulting TMA block were made and used for IHC analysis after transfer onto glass slides. Two colon tumor cell lines (CaCo-2, HT29) and one gastric tumor cell line (HGT1) were used as controls.

7) Immunohistochemical analysis

Anti-NM23 rabbit polyclonal antibody was purchased from Dako (Dako, Trappes, France) and used at 1:100 dilution. IHC was carried out on five-μm sections of tissue fixed in alcohol formalin for 24 h and included in paraffin. Sections were deparaffinized in histolemon (Carlo Erba Reagenti, Rodano, Italy), and were rehydrated in graded alcohol. Antigen enhancement was done by incubating the sections in target retrieval solution (Dako) as recommended by the manufacturer. The reactions were carried out using an automatic stainer (Dako Autostainer). Staining was done at room temperature as follows: after washes in phosphate buffer, followed by quenching of endogenous peroxidase activity by treatment with 3% H₂O₂, slides were first incubated with blocking serum (Dako) for 30 min and then with the affinity-purified antibody for one hour. After washes, slides were incubated with biotinylated antibody against rabbit IgG for 20 min., followed by streptadivin-conjugated peroxydase (Dako LSAB^{R}2 kit). Diaminobenzidine or 3-amino-9-ethylcarbazole was used as the chromogen. Slides were counter-stained with hematoxylin, and coverslipped using Aquatex (Merck, Darmstadt, Germany) mounting solution. The slides were evaluated under a light microscope by two pathologists. The results were expressed in terms of percentage (P) and intensity (I) of positive cells as previously described: results were scored by the quick score (Q) (Q=P×I). For the TMA, the mean of the score of two core biopsies minimum was done for each case. Correlations between status of sample (non-cancerous or cancer, and cancer with or without metastasis) or Kaplan-Meier MFS curves and IHC data were investigated by using Fisher exact test and Log-Rank test. Statistical tests were two-sided at the 5% level of significance.

### References

Agrawal D, Chen T, Irby R, Quackenbush J, Chambers A F, Szabo M, Cantor A, Coppola D and Yeatman T J. (2002). J Natl Cancer Inst, 94, 513-521.

Alizadeh A A, Eisen M B, Davis R E, Ma C, Lossos I S, Rosenwald A, Boldrick J C, Sabet H, Tran T, Yu X, Powell J I, Yang L, Marti G E, Moore T, Hudson J, Jr., Lu L, Lewis D B, Tibshirani R, Sherlock G, Chan W C, Greiner T C, Weisenburger D D, Armitage J O, Warnke R, Botstein D, Brown P O and Staudt L M. (2000). Nature, 403, 503-511.

Alon U, Barkai N, Notterman D A, Gish K, Ybarra S, Mack D and Levine A J. (1999). Proc Natl Acad Sci U S A, 96, 6745-6750.

Backert S, Gelos M, Kobalz U, Hanski M L, Bohm C, Mann B, Lovin N, Gratchev A, Mansmann U, Moyer M P, Riecken E O and Hanski C. (1999). Int J Cancer, 82, 868-874.

Beer D G, Kardia S L, Huang C C, Giordano T J, Levin A M, Misek D E, Lin L, Chen G, Gharib T G, Thomas D G, Lizyness M L, Kuick R, Hayasaka S, Taylor J M, Iannettoni M D, Orringer M B and Hanash S. (2002). Nat Med, 8, 816-824.

Bertucci F, Houlgatte R, Nguyen C, Viens P, Jordan B R and Birnbaum D. (2001). Lancet Oncol, 2, 674-682.

Bertucci F, Nasser V, Granjeaud S, Eisinger F, Adelaide J, Tagett R, Loriod B, Giaconia A, Benziane A, Devilard E, Jacquemier J, Viens P, Nguyen C, Birnbaum D and Houlgatte R. (2002). Hum Mol Genet, 11, 863-872.

Birkenkamp-Demtroder K, Christensen L L, Olesen S H, Frederiksen C M, Laiho P, Aaltonen L A, Laurberg S, Sorensen F B, Hagemann R and T F O R. (2002). Cancer Res, 62, 4352-4363.

Devilard E, Bertucci F, Trempat P, Bouabdallah R, Loriod B, Giaconia A, Brousset P, Granjeaud S, Nguyen C, Birnbaum D, Birg F, Houlgatte R and Xerri L. (2002). Oncogene, 21, 3095-3102.

Fearon E R and Vogelstein B. (1990). Cell, 61, 759-767.

Frederiksen C M, Knudsen S, Laurberg S and T F O R. (2003). J Cancer Res Clin Oncol, 15, 15.

Garber M E, Troyanskaya O G, Schluens K, Petersen S, Thaesler Z, Pacyna-Gengelbach M, van de Rijn M, Rosen G D, Perou C M, Whyte R I, Altman R B, Brown P O, Botstein D and Petersen I. (2001). Proc Natl Acad Sci U S A, 98, 13784-13789.

Kitahara O, Furukawa Y, Tanaka T, Kihara C, Ono K, Yanagawa R, Nita M E, Takagi T, Nakamura Y and Tsunoda T. (2001). Cancer Res, 61, 3544-3549.

Lin Y M, Furukawa Y, Tsunoda T, Yue C T, Yang K C and Nakamura Y. (2002). Oncogene, 21, 4120-4128.

Mohr S, Leikauf G D, Keith G and Rihn B H. (2002). J Clin Oncol, 20, 3165-3175.

Notterman D A, Alon U, Sierk A J and Levine A J. (2001). Cancer Res, 61, 3124-3130.

Singh D, Febbo P G, Ross K, Jackson D G, Manola J, Ladd C, Tamayo P, Renshaw A A, D'Amico A V, Richie J P, Lander E S, Loda M, Kantoff P W, Golub T R and Sellers W R. (2002). Cancer Cell, 1, 203-209.

Tureci O, Ding J, Hilton H, Bian H, Ohkawa H, Braxenthaler M, Seitz G, Raddrizzani L, Friess H, Buchler M, Sahin U and Hammer J. (2003). Faseb J, 17, 376-385.

Vogelstein B, Fearon E R, Hamilton S R, Kern S E, Preisinger A C, Leppert M, Nakamura Y, White R, Smits A M and Bos J L. (1988). N Engl J Med, 319, 525-532.

Williams N S, Gaynor R B, Scoggin S, Verma U, Gokaslan T, Simmang C, Fleming J, Tavana D, Frenkel E and Becerra C. (2003). Clin Cancer Res, 9, 931-946.

Zou T T, Selaru F M, Xu Y, Shustova V, Yin J, Mori Y. Shibata D, Sato F, Wang S, Olaru A, Deacu E, Liu T C, Abraham J M and Meltzer S J. (2002). Oncogene, 21, 4855-4862.

1. A method for analyzing differential gene expression associated with histopathologic features of colorectal disease, comprising the detection of the overexpression or underexpression of a pool of polynucleotide sequences from colon tissues, said pool comprising all or part of the polynucleotide sequences, or subsequences or complements thereof, selected from each of predefined polynucleotide sequence sets 1 through 644.

2. The method for analyzing differential gene expression associated with colon tumors according to claim 1, wherein the predefined polynucleotide sequence sets are selected from the group consisting of:
1; 4; 9; 10; 11; 13; 15; 16; 17; 18; 21; 27; 28; 30; 31; 34; 37; 39; 41; 43; 45; 46; 52; 53; 58; 59; 60; 65; 68; 69; 70; 75; 76; 78; 79; 80; 84; 85; 87; 88; 90; 95; 96; 98; 99; 101; 105; 108; 110; 111; 113; 114; 116; 119; 120; 122; 124; 125; 126; 127; 130; 131; 138; 139; 140; 141; 143; 150; 152; 153; 155; 159; 164; 171; 175; 176; 178; 181; 182; 184; 185; 189; 192; 196; 197; 198; 203; 205; 207; 208; 210; 213; 214; 215; 216; 218; 221; 223; 225; 227; 231; 235; 241; 243; 251; 256; 259; 261; 262; 263; 264; 266; 267; 268; 270; 279; 281; 286; 287; 288; 291; 298; 299; 301; 307; 310; 312; 313; 317; 319; 329; 331; 332; 337; 338; 339; 340; 341; 342; 344; 346; 352; 354; 357; 360; 361; 366; 368; 369; 377; 379; 381; 384; 385; 386; 390; 392; 394; 395; 397; 398; 400; 401; 405; 406; 409; 410; 413; 423; 427; 434; 436; 437; 438; 440; 442; 443; 444; 445; 448; 454; 459; 463; 464; 467; 469; 470; 488; 492; 495; 500; 503; 507; 508; 516; 518; 520; 522; 524; 538; 543; 547; 549; 552; 555; 557; 561; 567; 568; 569; 573; 574; 583; 586; 588; 592; 596; 597; 598; 599; 600; 601; 604; 609; 610; 611; 614; 616; 617; 621; 626; 627; 629; 630; 631; 632; 634; 635; 636; 638; 641; 642; and 644.

3. The method of claim 1, wherein the predefined polynucleotide sequence sets are selected from the group consisting of:
1; 9; 10; 16; 18; 27; 28; 30; 39; 41; 43; 45; 53; 58; 60; 65; 69; 75; 76; 113; 116; 120; 122; 126; 127; 130; 131; 138; 139; 140; 141; 143; 150; 152; 153; 159; 181; 182; 184; 189; 192; 197; 198; 210; 213; 214; 216; 218; 225; 227; 243; 259; 261; 264; 266; 267; 268; 281; 286; 287; 288; 291; 299; 307; 312; 313; 317; 319; 332; 337; 338; 339; 340; 341; 342; 344; 354; 357; 360; 361; 368; 381; 384; 385; 392; 394; 397; 398; 405; 423; 427; 442; 444; 464; 467; 469; 488; 495; 500; 507; 508; 516; 520; 522; 524; 538; 543; 547; 549; 552; 561; 567; 568; 569; 573; 586; 588; 592; 596; 600; 609; 614; 627; 629; 630; 635; 636; 641; 642; and 644.

4. The method of claim 1, wherein the predefined polynucleotide sequence sets are selected from the group consisting of:
4; 11; 13; 15; 17; 21; 31; 34; 37; 46; 52; 59; 68; 70; 78; 79; 80; 84; 85; 87; 88; 90; 95; 96; 98; 99; 101; 105; 108; 110; 111; 114; 119; 124; 125; 155; 164; 171; 175; 176; 178; 185; 196; 203; 205; 207; 208; 215; 221; 223; 231; 235; 241; 251; 256; 262; 263; 270; 279; 298; 301; 310; 329; 331; 346; 352; 366; 369; 377; 379; 386; 390; 395; 400; 401; 406; 409; 410; 413; 434; 436; 437; 438; 440; 443; 445; 448; 454; 459; 463; 470; 492; 503; 518; 555; 557; 574; 583; 597; 598; 599; 601; 604; 610; 611; 616; 617; 621; 626; 631; 632; 634; and 638.

5. The method of claim 1, wherein the predefined polynucleotide sequence sets are selected from the group consisting of:
2; 3; 10; 22; 24; 25; 30; 32; 33; 35; 36; 39; 40; 41; 42; 47; 50; 54; 57; 67; 72; 86; 97; 102; 103; 104; 107; 117; 118; 120; 128; 130; 132; 133; 134; 137; 144; 145; 146; 147; 149; 153; 156; 158; 162; 163; 165; 169; 170; 173; 174; 179; 180; 188; 191; 193; 194; 195; 199; 200; 201; 202; 204; 206; 209; 210; 211; 212; 213; 214; 216; 217; 219; 222; 234; 238; 246; 248; 249; 250; 255; 271; 272; 273; 276; 277; 278; 282; 283; 284; 291; 292; 293; 294; 295; 296; 303; 304; 305; 306; 308; 312; 314; 318; 323; 324; 325; 326; 330; 336; 337; 338; 339; 340; 341; 342; 343; 344; 347; 349; 350; 351; 353; 356; 359; 360; 361; 362; 363; 364; 371; 372; 374; 378; 380; 381; 382; 383; 384; 387; 388; 393; 396; 397; 399; 402; 403; 408; 414; 415; 417; 418; 419; 420; 421; 422; 426; 428; 430; 432; 433; 441; 446; 449; 457; 458; 460; 465; 471; 472; 473; 475; 476; 478; 480; 481; 482; 484; 485; 486; 490; 493; 494; 497; 501; 502; 504; 505; 509; 510; 514; 516; 520; 525; 526; 527; 528; 529; 530; 537; 538; 539; 541; 545; 546; 550; 558; 559; 560; 561; 562; 564; 565; 566; 571; 576; 577; 578; 580; 581; 584; 585; 586; 590; 591; 593; 594; 595; 596; 602; 607; 609; 612; 613; 615; 623; 624; 625; 633; 635; 639; 640; 643; and 644,
and wherein differential gene expression associated with visceral metastases in colon cancer is detected.

6. The method of claim 5, wherein the predefined polynucleotide sequence sets are selected from the group consisting of:
36; 86; 104; 107; 117; 132; 144; 153; 156; 174; 191; 209; 248; 349; 350; 396; 417; 419; 432; 558; 566; 613; 623; 625; 633; and 643.

7. The method of claim 5, wherein the predefined polynucleotide sequence sets are selected from the group consisting of:
2; 3; 10; 22; 24; 25; 30; 32; 33; 35; 39; 40; 41; 42; 47; 50; 54; 57; 67; 72; 97; 102; 103; 118; 120; 128; 130; 133; 134; 137; 145; 146; 147; 149; 158; 162; 163; 165; 169; 170; 173; 179; 180; 188; 193; 194; 195; 199; 200; 201; 202; 204; 206; 210; 211; 212; 213; 214; 216; 217; 219; 222; 234; 238; 246; 249; 250; 255; 271; 272; 273; 276; 277; 278; 282; 283; 284; 291; 292; 293; 294; 295; 296; 303; 304; 305; 306; 308; 312; 314; 318; 323; 324; 325; 326; 330; 336; 337; 338; 339; 340; 341; 342; 343; 344; 347; 351; 353; 356; 359; 360; 361; 362; 363; 364; 371; 372; 374; 378; 380; 381; 382; 383; 384; 387; 388; 393; 397; 399; 402; 403; 408; 414; 415; 418; 420; 421; 422; 426; 428; 430; 433; 441; 446; 449; 457; 458; 460; 465; 471; 472; 473; 475; 476; 478; 480; 481; 482; 484; 485; 486; 490; 493; 494; 497; 501; 502; 504; 505; 509; 510; 514; 516; 520; 525; 526; 527; 528; 529; 530; 537; 538; 539; 541; 545; 546; 550; 559; 560; 561; 562; 564; 565; 571; 576; 577; 578; 580; 581; 584; 585; 586; 590; 591; 593; 594; 595; 596; 602; 607; 609; 612; 615; 624; 635; 639; 640; and 644.

8. The method of claim 1, wherein the predefined polynucleotide sequence sets are selected from the group consisting of:
38; 55; 66; 91; 93; 102; 103; 133; 142; 144; 153; 163; 190; 210; 232; 254; 280; 296; 300; 304; 311; 321; 335; 378; 383; 384; 420; 425; 429; 432; 468; 473; 487; 516; 519; 544; 553; 573; 577; 578; 585; 587; 589; 592; 605; 608; and 644,
and wherein differential expression of genes associated with lymph node metastases in colon cancer is detected.

9. The method of claim 8, wherein the predefined polynucleotide sequence sets are selected from the group consisting of:
55; 66; 144; 153; 432; 553; and 608.

10. The method of claim 8, wherein the predefined polynucleotide sequence sets are selected from the group consisting of:
38; 91; 93; 102; 103; 133; 142; 163; 190; 210; 232; 254; 280; 296; 300; 304; 311; 321; 335; 378; 383; 384; 420; 425; 429; 468; 473; 487; 516; 519; 544; 573; 577; 578; 585; 587; 589; 592; 605; and 644.

11. The method of claim 1, wherein the predefined polynucleotide sequence sets are selected from the group consisting of:
29; 48; 56; 62; 71; 77; 82; 109; 112; 135; 136; 154; 157; 166; 167; 186; 220; 226; 236; 237; 239; 240; 242; 244; 253; 260; 277; 290; 297; 348; 358; 375; 376; 404; 407; 412; 416; 424; 431; 450; 451; 452; 462; 474; 477; 479; 486; 498; 511; 521; 533; 534; 535; 542; 572; 619; and 622,
and wherein differential gene expression associated with MSI phenotype in colon cancer is detected.

12. The method of claim 11, wherein the predefined polynucleotide sequence sets are selected from the group consisting of:
48; 56; 62; 157; 186; 220; 226; 253; 260; 376; 450; 452; 462; 498; and 511.

13. The method of claim 11, wherein the predefined polynucleotide sequence sets are selected from the group consisting of:
29; 71; 77; 82; 109; 112; 135; 136; 154; 166; 167; 236; 237; 239; 240; 242; 244; 277; 290; 297; 348; 358; 375; 404; 407; 412; 416; 424; 431; 451; 474; 477; 479; 486; 521; 533; 534; 535; 542; 572; 619; and 622.

14. The method of claim 1, wherein the predefined polynucleotide sequence sets are selected from the group consisting of:
6; 19; 43; 49; 83; 89; 94; 100; 151; 168; 172; 177; 224; 252; 258; 265; 309; 315; 316; 320; 322; 328; 355; 365; 391; 443; 453; 455; 466; 483; 496; 499; 506; 512; 513; 515; 517; 531; 532; 554; 563; 575; 579; 606; 618; and 637,
and wherein differential gene expression associated with the location of a primary colorectal carcinoma in colon cancer is detected.

15. The method of claim 14, wherein the predefined polynucleotide sequence sets are selected from the group consisting of:
19; 43; 89; 94; 100; 168; 224; 309; 328; 355; 391; 466; 531; 532; 563; and 637.

16. The method of claim 14, wherein the predefined polynucleotide sequence sets are selected from the group consisting of:
6; 49; 83; 151; 172; 177; 252; 258; 265; 315; 316; 320; 322; 365; 443; 453; 455; 483; 496; 499; 506; 512; 513; 515; 517; 554; 575; 579; 606; and 618.

17. The method of claim 1, wherein the predefined polynucleotide sequence sets are selected from the group consisting of:
2; 3; 5; 7; 8; 10; 12; 14; 20; 22; 23; 26; 28; 32; 33; 35; 36; 41; 42; 44; 47; 50; 51; 60; 61; 63; 64; 70; 73; 74; 81; 92; 93; 95; 106; 115; 118; 120; 121; 123; 129; 130; 132; 133; 137; 145; 148; 149; 160; 161; 162; 163; 183; 187; 188; 195; 199; 200; 202; 206; 209; 211; 213; 214; 217; 219; 222; 228; 229; 230; 233; 234; 238; 245; 246; 247; 250; 257; 269; 271; 274; 275; 276; 282; 283; 284; 285; 289; 291; 292; 296; 302; 303; 304; 312; 314; 318; 323; 327; 333; 334; 335; 336; 337; 339; 340; 341; 342; 344; 345; 347; 350; 351; 356; 359; 361; 362; 363; 364; 367; 370; 373; 374; 378; 380; 381; 382; 383; 384; 387; 389; 402; 403; 408; 411; 414; 418; 420; 428; 430; 433; 435; 439; 444; 446; 447; 449; 456; 457; 458; 460; 461; 465; 473; 478; 482; 484; 489; 490; 491; 494; 497; 501; 502; 504; 510; 514; 516; 520; 523; 528; 529; 530; 536; 537; 538; 539; 540; 548; 551; 556; 561; 562; 570; 571; 580; 581; 582; 584; 586; 590; 591; 593; 594; 596; 603; 607; 609; 612; 615; 620; 624; 625; 628; 635; 639; and 640,
and wherein differential expression associated with the survival and death of subjects with colon cancer is detected.

18. The method of claim 17, wherein the predefined polynucleotide sequence sets are selected from the group consisting of:
5; 14; 36; 44; 61; 64; 70; 81; 95; 115; 121; 132; 183; 209; 228; 275; 333; 334; 350; 367; 373; 435; 439; 523; 570; 603; and 625.

19. The method of claim 17, wherein the predefined polynucleotide sequence sets are selected from the group consisting of:
2; 3; 7; 8; 10; 12; 20; 22; 23; 26; 28; 32; 33; 35; 41; 42; 47; 50; 51; 60; 63; 73; 74; 92; 93; 106; 118; 120; 123; 129; 130; 133; 137; 145; 148; 149; 160; 161; 162; 163; 187; 188; 195; 199; 200; 202; 206; 211; 213; 214; 217; 219; 222; 229; 230; 233; 234; 238; 245; 246; 247; 250; 257; 269; 271; 274; 276; 282; 283; 284; 285; 289; 291; 292; 296; 302; 303; 304; 312; 314; 318; 323; 327; 335; 336; 337; 339; 340; 341; 342; 344; 345; 347; 351; 356; 359; 361; 362; 363; 364; 370; 374; 378; 380; 381; 382; 383; 384; 387; 389; 402; 403; 408; 411; 414; 418; 420; 428; 430; 433; 444; 446; 447; 449; 456; 457; 458; 460; 461; 465; 473; 478; 482; 484; 489; 490; 491; 494; 497; 501; 502; 504; 510; 514; 516; 520; 528; 529; 530; 536; 537; 538; 539; 540; 548; 551; 556; 561; 562; 571; 580; 581; 582; 584; 586; 590; 591; 593; 594; 596; 607; 609; 612; 615; 620; 624; 628; 635; 639; and 640.

20. The method of claim 1, wherein the predefined polynucleotide sequence are 1; 4; 15; 21; 27; 58; 68; 75; 79; 95; 98; 101; 114; 119; 127; 131; 140; 155; 176; 192; 241; 243; 259; 263; 270; 279; 286; 298; 299; 307; 310; 312; 313; 317; 329; 346; 357; 360; 361; 394; 395; 398; 405; 406; 413; 427; 436; 437; 438; 443; 454; 464; 507; 522; 547; 552; 555; 568; 569; 614; 631; 634; 636; 641; and 644.

21. The method of claim 1 wherein the predefined polynucleotide sequence sets are 32; 33; 50; 133; 188; 217; 271; 284; 296; 303; 312; 323; 340; 343; 361; 403; 408; 473; 484; 494; 502; 516; and 624.

22. The method of claim 1, wherein the predefined polynucleotide sequence sets are 142; 144; 153; 190; 280; 468; 553; and 589.

23. The method of claim 1, wherein the predefined polynucleotide sequence sets are 29; 62; 71; 109; 136; 154; 348; 404; 412; 416; 431; 451; 479; 486; 498; 535 and 622.

24. The method of claim 1, wherein the predefined polynucleotide sequence sets are 109; 154; 412; 486; 535 and 622.

25. The method of claim 1, wherein the predefined polynucleotide sequence sets are 10; 12; 33; 214; 217; 271; 344; 383; 387; 414; 473; 484; 516; 536; and 561.

26. The method of claim 1, wherein the predefined polynucleotide sequence sets are 43; 100; 151; 172; 265; 315; 443; 499; 532 and 554.

27. The method of claim 1, wherein said detection of over expression or under expression of polynucleotide sequences is carried out by FISH or IHC.

28. The method of claim 1, wherein said detection is performed on nucleic acids from a tissue sample.

29. The method of claim 1, wherein said detection is performed on nucleic acids from a tumor cell line.

30. The method of claim 1, wherein said detection is performed on DNA microarrays.

31. A method or prognosis or diagnosis of colon cancer, or for monitoring the treatment of a subject with a colon cancer, comprising:
1) obtaining colon tissue polynucleotide sequences from a subject; and
2) analyzing the colon tissue polynucleotide sequences by detecting the overexpression or underexpression of a pool of polynucleotide sequences, said pool comprising all or part of the polynucleotide sequences, or subsequences or complements thereof, selected from each of predefined polynucleotide sequnce sets 1 through 644.

32. A method for differentiating a normal cell from a cancer cell, comprising:
1) obtaining polynucleotide sequences from normal and cancer cells; and
2) analyzing the polynucleotide sequences from step 1) by detecting the overexpression or underexpression of a pool of polynucleotide sequences, said pool comprising all or part of the polynucleotide sequences, or subsequences or complements thereof, selected from each of predefined polynucleotide sequnce sets 1 through 644.

33. A polynucleotide library, comprising a pool of polynucleotide sequences either overexpressed or underexpressed in colon tissue or cells, said pool corresponding to all or part of the polynucleotide sequences of SEQ ID Nos. 1 through 1596, or subsequences or complements thereof.

34. A polynucleotide library according to claim 33, immobilized on a solid support.

35. A polynucleotide library according to claim 34, wherein the solid support is selected from the group consisting of nylon membrane, nitrocellulose membrane, glass slide, glass beads, membranes on glass support and silicon chip.

36. A method of detecting differential gene expression, comprising:
1) obtaining a test sample comprising polynucleotide sequences from a subject,
2) reacting the test sample obtained in step (1) with a polynucleotide library according to claim 33, and
3) detecting the reaction product of step (2).

37. The method of claim 36, wherein the test sample is labeled before reaction step (2).

38. The method of claim 37, wherein the label is selected from the group consisting of radioactive, calorimetric, enzymatic, molecular amplification, bioluminescent and fluorescent labels.

39. The method of claim 36, further comprising:
4) obtaining a control sample comprising polynucleotide sequences;
5) reacting the control sample with said polynucleotide library;
6) detecting a control sample reaction product; and
7) comparing the amount of the test sample reaction product to the amount of the control sample reaction product.

40. The method of claim 36, wherein the test sample comprises cDNA, RNA or mRNA.

41. The method of claim 40, wherein mRNA is isolated from the test sample and cDNA is obtained by reverse transcription of said mRNA.

42. The method of claim 36, wherein said reaction step is performed by hybridizing the test sample with the polynucleotide library.

43. The method of claim 36, wherein conditions associated with colorectal cancer are detected, diagnosed, staged, classified, monitored, predicted, prevented or treated.

44. A method of assigning a therapeutic regimen to subject who has histopathological features of colorectal disease, comprising:
1) detecting the overexpression or underexpression of a pool of polynucleotide sequences from colon tissues, said pool comprising all or part of the polynucleotide sequences, or subsequences or complements thereof, selected from each of predefined polynucleotide sequence sets 1 through 644;
2) classifying said subject as having a “poor prognosis” or a “good prognosis” on the basis of the the overexpression or underexpression detected in step (1);
3) assigning said subject a therapeutic regimen, said therapeutic regimen (i) comprising no adjuvant chemotherapy if the patient is lymph node negative and is classified as having a good prognosis, or (ii) comprising chemotherapy if said patient has any other combination of lymph node status and expression profile.

45. The method of claim 44, wherein the assigning of a therapeutic regimen comprises the use of an appropriate dose of irinotecan.

46. The method of claim 45, wherein the dose of irinotecan is selected according to the presence or the absence of a polymorphism in a uridine diphosphate glucuronosyltransferase I (UGT1A1) gene promoter of the subject.

47. The method of claim 46, wherein the polymorphism is the presence of an abnormal number of (TA) repeats in the sequence of said promoter.
